(19) United States
(12) Patent Application Publication
(10) Pub. No.: US 2022/0135642 A1
(43) Pub. Date: May 5, 2022

(54) CELLS, COMPOSITIONS AND METHODS FOR ENHANCING IMMUNE FUNCTION

(71) Applicant: THE COUNCIL OF THE QUEENSLAND INSTITUTE OF MEDICAL RESEARCH, Herston (AU)

(72) Inventors: Tobias Bald, Herston (AU); Matthias Braun, Herston (AU); Mark Smyth, Herston (AU)

(21) Appl. No.: 17/434,486

(22) PCT Filed: Feb. 27, 2020
(86) PCT No.: PCT/AU2020/050176

§ 371 Date: Aug. 27, 2021

(30) Foreign Application Priority Data
Feb. 27, 2019 (AU) 2019900621

(51) Int. Cl. C07K 14/705; A61P 35/00; C07K 14/725; A61K 35/17; A61K 45/06; G01N 33/50; C12N 5/0783; G01N 33/574

(52) U.S. Cl. CPC C07K 14/70503; A61P 35/00; C07K 14/7051; A61K 35/17; G01N 2333/70503; G01N 33/5091; C12N 5/0636; G01N 33/57492; C12N 2510/00; A61K 45/06

(57) ABSTRACT

The present disclosure relates generally to polypeptides, cells, compositions and methods for enhancing immune function, and in particular the immune function of T cells, such as CD8+ T cells. More particularly, the present invention relates to modified DNAM-1 polypeptides, T cells expressing recombinant and/or modified DNAM-1, and methods of using these cells in adoptive T cell transfer, such as for the treatment of cancer or infection. The disclosure also relates to methods for preparing T cells with enhanced immune function; methods for preparing T cells for adoptive cell therapy; methods for assessing the immune function of T cells in a subject or cell population; methods for predicting the responsiveness of a subject with cancer to cancer therapy; and methods for predicting the survival or survival time of a subject with cancer.

### RELATED APPLICATIONS

This application claims priority to Australian Provisional Application No. 2019900621 entitled “Cells, Compositions and Methods for Enhancing Immune Function” filed 27 Feb. 2019, the content of which is incorporated herein by reference in its entirety.

### FIELD OF THE INVENTION

The present disclosure relates generally to polypeptides, cells, compositions and methods for enhancing immune function, and in particular the immune function of T cells, such as CD8+ T cells. More particularly, the present invention relates to modified DNAM-1 polypeptides, T cells expressing recombinant and/or modified DNAM-1, and methods of using these cells in adoptive T cell transfer, such as for the treatment of cancer or infection. The disclosure also relates to methods for preparing T cells with enhanced immune function; methods for preparing T cells for adoptive cell therapy; methods for assessing the immune function of T cells in a subject or cell population; methods for predicting the responsiveness of a subject with cancer to cancer therapy; and methods for predicting the survival or survival time of a subject with cancer.

### BACKGROUND OF THE INVENTION

Cancer therapies have evolved significantly in recent decades. Not only have the traditional treatments of surgery, radiation therapy and chemotherapy become more precise, but newer treatments are also providing a broad range of therapies that target specific molecules, cells and/or pathways associated with cancer development, and which may be particularly effective for different cancers, stages of cancer, and/or populations. Thus, in addition to surgery, radiation therapy and traditional chemotherapy (i.e. non-targeted chemotherapy that involves the use of drugs to kill rapidly dividing cells such as cancer), cancer therapies now also include, for example, hormone therapy, targeted therapy and immunotherapy.

Cancer immunotherapy functions by exploiting or utilizing the immune system of the patient to treat the cancer. This can be through several mechanisms and by using different strategies, including non-specific stimulation of immune responses by stimulating effector cells and/or inhibiting regulatory cells (e.g. by administration of cytokines such as IL-2 and IFN-γ, or drugs such as thalidomide (Thalomid®), ienalidomide (Revlimid®), pomalidomide (Pomalyst®) and miquimod (Zyclara®)), active immunization to stimulate or enhance specific anti-cancer immune responses (e.g. using cancer vaccines such as the HPV vaccines Gardasil® and Cevarix® for the prevention of cervical cancer, Sipuleucel-T (Provenge®) for the treatment of prostate cancer, and the Bacillus Calmette-Guérin (BCG) vaccine for the treatment of bladder cancer), and the passive transfer of antibodies or the passive transfer of activated immune cells (i.e. adoptive cell therapy (ACT), e.g. chimeric antigen receptor (CAR) T cell therapy). Antibodies that have been developed as cancer immunotherapies include, for example, immune checkpoint inhibitor antibodies (e.g. targeting CTLA-4, PD-1 or PD-L1) and antibodies that target molecules on cancer cells so as to induce an immune response to the cancer cell (e.g. anti-CD52 antibodies).

However, while cancer immunotherapies have provided an expanded tool box for cancer treatment and can be very effective in some patients, many patients do not benefit from currently approved cancer immunotherapies. Beside primary unresponsiveness, many patients acquire resistance to current immune checkpoint blocking antibodies. Thus, there remains a need for methods and compositions that promote immune function and enhance the effectiveness of immunotherapies, such as cancer immunotherapies and other immunotherapies.

### SUMMARY OF THE INVENTION

The present disclosure arises from the unexpected finding that DNAX accessory molecule-1 (DNAM-1; CD226) is essential for T cell function in tumors. The inventors determined that DNAM-1 signaling induces downregulation of DNAM-1 and limits or reduces anti-tumor activity of the T cells. In contrast, maintaining or increasing surface expression of DNAM-1 enhances the function of T cells and increases anti-tumor activity. Maintaining or increasing surface expression can be effected by, for example, targeting (e.g. mutating or abolishing) the tyrosine at position 322 of human DNAM-1, targeting (e.g. mutating or abolishing) the AP2 binding motif at positions 324-327 of human DNAM-1; targeting (e.g. mutating or abolishing) the AP-2 binding motif at positions corresponding to positions 282-287 of human DNAM-1; targeting (e.g. mutating or abolishing) the Cbl-b binding motif at positions 320-323 of human DNAM-1; and/or targeting (e.g. mutating or abolishing) the lysine(s) at position at 295 and/or 333 of human DNAM-1 (with numbering relative to the precursor human DNAM-1 set forth in SEQ ID NO:1). Thus, modified DNAM-1 polypeptides having or more modifications that target the polypeptide in this way can exhibit increased cell surface retention or expression when expressed in a T cell compared to a wild-type DNAM-1 polypeptide expressed in a T cell (e.g. an endogenous wild-type DNAM-1 polypeptides expressed in a T cell. This has significant implications for cancer therapy, and in particular cancer immunotherapies. Specifically, and as described herein for the first time, T cells, including CAR T cells, expressing recombinant and/or modified DNAM-1 polypeptides of the present disclosure can be adoptively transferred to a subject to treat cancer in the subject, either as a standalone treatment or in combination with other cancer therapies. T cells that express endogenous DNAM-1 (including high levels of endogenous DNAM-1) can also be isolated for subsequent adoptive transfer to a subject with cancer. The enhanced immune function of T cells expressing DNAM-1 can also be exploited for the treatment of infection in a subject. T cells expressing recombinant and/or modified DNAM-1 polypeptides of the present disclosure can be adoptively transferred to a subject to treat infection in the subject, either as a standalone treatment or in combination with other therapies. T cells that express endogenous DNAM-1 can also be isolated for subsequent adoptive transfer to a subject with an infection for treatment of the infection. As demonstrated herein, DNAM-1 is also a biomarker for T cell immune function, cancer survival and responsiveness to cancer therapy.

Accordingly, in one aspect, provided herein are T cells, comprising a modified DNAM-1 polypeptide, wherein the modified DNAM-1 polypeptide exhibits increased retention on the surface of the cell, or increased cell surface expression, compared to a wild-type DNAM-1 polypeptide; and wherein the T cell is a human T cell.

In some embodiments, the modified DNAM-1 polypeptide comprises a modification of a tyrosine at a position corresponding to position 322 of SEQ ID NO:1. The modification may be, for example, an amino acid substitution or deletion, such as a substitution of the tyrosine with a phenylalanine.

In further embodiments, the modified DNAM-1 polypeptide comprises a modification of the AP-2 binding motif YXXF at positions corresponding to positions 325-328 of SEQ ID NO:1, wherein the modification abolishes the AP-2 binding motif YXXF. For example, the modified DNAM-1 polypeptide may comprise an amino acid substitution or deletion of the tyrosine at the position corresponding to position 325 of SEQ ID NO:1; an amino acid substitution or deletion of the phenylalanine at the position corresponding to position 328 of SEQ ID NO:1; an amino acid insertion after any one of the positions corresponding to position 325, 326 or 327 of SEQ ID NO:1; and/or deletion of one or more of the residues at positions corresponding to positions 326 and 327.

In other embodiments, the modified DNAM-1 polypeptide comprises a modification of the AP-2 binding motif EXXXLF at positions corresponding to positions 282-287 of SEQ ID NO:1, wherein the modification abolishes the AP-2 binding motif EXXXLF. For example, the modified DNAM-1 polypeptide may comprise an amino acid substitution or deletion of the glutamic acid at the position corresponding to position 282 of SEQ ID NO:1; an amino acid substitution or deletion of the leucine at the position corresponding to position 286 of SEQ ID NO; an amino acid substitution or deletion of the phenylalanine at the position corresponding to position 287 of SEQ ID NO:1; an amino acid insertion after any one or more of the residues at positions corresponding to 282-286 of SEQ ID NO:1; and/or a deletion of one or more of the residues at positions corresponding to positions 283, 284 and 285 of SEQ ID NO:1.

In further embodiments, the modified DNAM-1 polypeptide comprises a modification of the Cbl-b binding motif ((D/N)XpY) at positions corresponding to positions 320-322 of SEQ ID NO:1, wherein the modification abolishes the Cbl-6 binding motif. In some examples, the modified DNAM-1 polypeptide comprises an amino acid deletion or substitution of the aspartic acid at the position corresponding to position 320 of SEQ ID NO:1; and/or an amino acid insertion after the position corresponding to position 320 and/or 321 of SEQ ID NO:1.

In other embodiments, the modified DNAM-1 polypeptide comprises an amino acid substitution or deletion of the lysine at the position corresponding to position 295; and/or an amino acid substitution or deletion of the lysine at the position corresponding to position 333 of SEQ ID NO:1.

In further aspects, the present disclosure provides a T cell, comprising a recombinant DNAM-1 polypeptide, wherein the recombinant DNAM-1 polypeptide is not fused to, or does not comprise, a heterologous intracellular signalling domain; and the T cell comprises an endogenous T cell receptor (TCR). In some embodiments, the DNAM-1 polypeptide comprises modification of a tyrosine at a position corresponding to position 322 of SEQ ID NO:1. In additional aspects, provided is a T cell, comprising a recombinant DNAM-1 polypeptide that comprises a modification of the tyrosine at a position corresponding to position 322 of SEQ ID NO:1, wherein the T cell is a human T cell. The modification of the tyrosine at the position corresponding to position 322 of SEQ ID NO:1 in these aspects may be an amino acid substitution or a deletion, e.g. a substitution of the tyrosine with a phenylalanine.

In some embodiments of each of the aspects related to a T cell as described above, the DNAM-1 polypeptide lacks all or a portion of the cytoplasmic domain. In other embodiments, the DNAM-1 polypeptide comprises all or a portion of the extracellular domain; the IgG1 domain; and/or the IgG2 domain.

In particular examples, the DNAM-1 polypeptide comprises a sequence of amino acids set forth in any one of SEQ ID NOs:5-9 or 21-30, or a sequence having at least or about 90%, 91%, 92%, 93%, 94%, 95%, 96%, 97%, 98% or 99% sequence identity thereto, wherein the DNAM-1 polypeptide does not comprise the same sequence as a wild-type DNAM-1 polypeptide (i.e. has less than 100% sequence identity to a wild-type DNAM-1 polypeptide, such as a wild-type human DNAM-1 polypeptide (e.g. one set forth in SEQ ID NO:1 or 2).

In some embodiments, the T cell is a CD8⁺ T cell. In other embodiments, the T cell is a CD4⁺ T cell. The T cell may be an αβ T cell or a γδ T cell. In particular examples, the T cell is derived from primary human PBMCs isolated from a human subject.

In some embodiments, the T cell comprises a recombinant TCR and/or a chimeric antigen receptor (CAR), e.g. the T cell can be a CAR-T cell. In some examples, the CAR binds to a tumor antigen selected from among TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-1Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-Ia, MAGE-A1, legumain, HPV E6, E7, MAGEA1, ETV6-AML, sperm protein 17, XAGE1, Tie 2, MAD-CT-1, MAD-CT-2, Fos-related antigen 1, p53, p53 mutant, prostein, survivin and telomerase, PCTA-I/Galectin 8, MelanA/MART-1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRLS, and IGLL1.

Also provided are pharmaceutical compositions, comprising a T cell of the present disclosure, and a pharmaceutically acceptable carrier. The pharmaceutical composition can further comprise a chemotherapeutic agent (e.g. an immune checkpoint inhibitor, such as a CTLA-4, PD-1 or PD-L1 inhibitor) or an anti-infective agent (e.g. an antibiotic, amebicide, antifungal, antiprotozoal, antimalarial, antituberculotic or antiviral).

A further aspect of the disclosure relates to a method for preparing a T cell population for adoptive cell therapy, comprising: obtaining a sample of T cells from a subject; selecting DNAM+ T cells from the sample; and expanding the DNAM+ T cells to produce a T cell population for adoptive T cell therapy. In some embodiments, the method comprises selecting DNAM+ CD8+ T cells and/or DNAM+ CD4+ T cells. In particular examples, the method further comprises engineering the DNAM+ T cells to express a CAR or a transgenic TCR. Also provided therefore is a T cell population produced by such a method.

In another aspect, provided is a method of increasing immune function in a subject, comprising administering to the subject a T cell of the present disclosure (e.g. a T cell expressing a recombinant and/or modified DNAM-1 polypeptide as described herein), a pharmaceutical composition of the present disclosure, or a T cell population of the present disclosure.

In a further aspect, provided is a method for treating cancer in a subject, comprising administering to the subject a T cell of the present disclosure (e.g. a T cell expressing a recombinant and/or modified DNAM-1 polypeptide as described herein), a pharmaceutical composition of the present disclosure, or a T cell population of the present disclosure. In some examples, the method further comprises administering a chemotherapeutic agent to the subject (e.g. an immune checkpoint inhibitor, such as a CTLA-4, PD-1 or PD-L1 inhibitor). In some examples, wherein the cancer is skin cancer (e.g., melanoma), lung cancer, breast cancer, ovarian cancer, gastric cancer, bladder cancer, pancreatic cancer, endometrial cancer, colon cancer, kidney cancer, esophageal cancer, prostate cancer, colorectal cancer, glioblastoma, head and neck cancer, neuroblastoma, or hepatocellular carcinoma. In further examples, the cancer is resistant to one or more immune checkpoint inhibitors prior to administration of the T cell or pharmaceutical composition. In particular embodiments, the T cell is autologous. In other embodiments, the T cell is allogeneic.

In another aspect, provided is a method for treating an infection in a subject, comprising administering to the subject a T cell of the present disclosure (e.g. a T cell expressing a recombinant and/or modified DNAM-1 polypeptide as described herein), a pharmaceutical composition of the present disclosure, or a T cell population of the present disclosure. In some embodiments, the infection is with virus and/or bacteria. The infection may be an acute infection or a chronic infection. In some embodiments, the method further comprises administering an anti-infective agent to the subject (e.g. an antibiotic, amebicide, antifungal, antiprotozoal, antimalarial, antituberculotic or antiviral). In particular embodiments, the T cell is autologous. In other embodiments, the T cell is allogeneic.

Also provided is use of a T cell of the present disclosure (e.g. a T cell expressing a recombinant and/or modified DNAM-1 polypeptide as described herein), a pharmaceutical composition of the present disclosure, or a T cell population of the present disclosure for the preparation of a medicament for treating cancer, treating an infection and/or enhancing immune function in a subject.

A further aspect of the disclosure relates to a method for assessing the immune function of a T cell or a population of T cells in a subject, comprising assessing the amount or level of DNAM-1 on the surface of a T cell or T cells in population of T cells in a sample from the subject and comparing the amount or level of DNAM-1 on the surface of the T cell or T cells in the population of T cells in the sample from the subject to the amount or level of DNAM-1 on the surface of a T cell or a population of T cells in a control sample. In some examples, assessing the amount or level of DNAM-1 on the surface of T cells in a population of T cells in a sample comprises detecting the number or percentage of DNAM-1+ T cells in the sample. In one embodiment, the control sample comprises T cells with normal or effective immune function, and a reduced amount or level of DNAM-1 on the surface of a T cell or T cells in a population of T cells in the sample from the subject compared to the amount or level of DNAM-1 on the surface of a T cell in the control sample indicates that the immune function of the T cell or a population of T cells in the subject is impaired or ineffective. In additional embodiments, the method comprises obtaining a sample from the subject, wherein the sample comprises a T cell or population of T cells; contacting the sample with a binding agent that binds to DNAM-1 on the surface of a T cell (e.g. an anti-DNAM-1 antibody); and detecting the binding agent when bound to the T cell or to T cells in the population of T cells to thereby assess the amount or level of DNAM-1 on the surface of the T cells, or the number of percentage of DNAM+ T cells, in the sample from the subject. In some examples, the subject has cancer or has an infection. In some embodiments, the subject is further administered a therapy, such as a chemotherapeutic agent or an anti-infective agent.

Another aspect of the disclosure relates to a method for predicting the likelihood that a subject with cancer will respond to therapy with an immune checkpoint inhibitor, comprising detecting the number or percentage of DNAM-1+ CD8+ T cells in a sample (e.g. a tumour sample, such that the T cells are tumour infiltrating T cells) from the subject, and comparing the number or percentage of DNAM-1+ CD8+ cells in the sample from the subject to a reference level or amount. In a particular embodiment, DNAM-1+ CD8+ T cells as a percentage of total CD8+ T cells in the sample is detected. In some embodiments, where the subject is predicted to be responsive to therapy, the subject is further administered a therapy an immune checkpoint inhibitor. In some embodiments, where the subject is predicted to be non-responsive to therapy, the subject is administered a therapy to improve responsiveness, e.g. a T cell of the present disclosure.

Also provided is a modified DNAM-1 polypeptide comprising a modification of the AP-2 binding motif YXXF at positions corresponding to positions 325-328 of SEQ ID NO:1, wherein the modification abolishes the AP-2 binding motif YXXF. In some examples, the DNAM-1 polypeptide comprises an amino acid substitution or deletion of the tyrosine at the position corresponding to position 325 of SEQ ID NO:1; an amino acid substitution or deletion of the phenylalanine at the position corresponding to position 328 of SEQ ID NO:1; an amino acid insertion after any one of the positions corresponding to position 325, 326 or 327 of SEQ ID NO:1; and/or a deletion of one or more of the residues at positions corresponding to positions 326 and 327.

In another aspect, provided is a modified DNAM-1 polypeptide comprising a modification of the AP-2 binding motif EXXXLF at positions corresponding to positions 282-287 of SEQ ID NO:1, wherein the modification abolishes the AP-2 binding motif EXXXLF. In some examples, the modified DNAM-1 polypeptide comprises an amino acid substitution or deletion of the glutamic acid at the position corresponding to position 282 of SEQ ID NO:1; an amino acid substitution or deletion of the leucine at the position corresponding to position 286 of SEQ ID NO; an amino acid substitution or deletion of the phenylalanine at the position corresponding to position 287 of SEQ ID NO:1; an amino acid insertion after any one or more of the residues at positions corresponding to 282-286 of SEQ ID NO:1; and/or a deletion of one or more of the residues at positions corresponding to positions 283, 284 and 285.

In a further aspect, provided is a modified DNAM-1 polypeptide comprising a modification of the Cbl-B binding motif ((D/N)XpY) at positions corresponding to positions 320-322 of SEQ ID NO:1, wherein the modification abolishes the Cbl-b binding motif. In one example, the DNAM-1 polypeptide comprises an amino acid deletion or substitution of the aspartic acid at the position corresponding to position 320 of SEQ ID NO:1; and/or an amino acid insertion after the position corresponding to position 320 and/or 321 of SEQ ID NO:1.

In another aspect, provided is a modified DNAM-1 polypeptide comprising a modification (e.g. an amino acid substitution or deletion) of the lysine at the position corresponding to position 295 and/or the lysine at the position corresponding to position 333 of SEQ ID NO:1.

In particular embodiments, the modified DNAM-1 polypeptides of the present disclosure have increased cell surface expression or retention when expressed in T cell compared to a wild-type DNAM-1 polypeptide when expressed in a T cell. In some embodiments, the modification is relative to the wild-type human DNAM-1 polypeptide set forth in SEQ ID NO:1 or 2.

### BRIEF DESCRIPTION OF THE DRAWINGS

FIG. 1 is a graphical representation showing tumor growth C57BL/6J (WT) mice and DNAM-1 deficient (CD226^{KO}) mice. A. Mean tumor growth curves of B16F10 melanoma in WT or CD226KO mice (n=7 per group, mean±SEM, representative results of two experiments are shown). B. Mean tumor growth curves of MC38 in WT or CD226^{KO} mice (n=8 per group, mean±SEM, representative results of two experiments are shown). C. Mean tumor growth curves of MC38-OVA^{dim} in WT or CD226^{KO} mice (n=7-9 per group, mean±SEM, representative results of two experiments are shown). D. Mean tumor growth curves of MC38-OVA^{hi} in WT or CD226^{KO} mice (n=7-10 per group, mean±SEM, representative results of two experiments are shown). *=p<0.05, **=p<0.01, ****=p<0.0001, student's t-test.

FIG. 2 shows representative flow cytometric histograms showing CD226 expression on CD8+ TILs from tumour bearing WT mice (left panel and corresponding quantification of CD226 subsets (right panel, n=15, mean±SD, cumulative results of three experiments are shown). A. B16F10 tumours. B. MC38 tumours. C. MC38-OVA^{dim} tumours. D. MC38-OVA^{hi} tumours.

FIG. 3 shows corresponding flow cytometric quantification of IFN-γ+, TNF-α+ Granzyme-B+ and Ki67+ CD8+ TILs in tumours in WT mice across indicated CD226 subsets. A. B16F10 tumours (n=15 for IFN-γ and TNF-α, n=12 for GrzB, n=10 for Ki67, cumulative results of two experiments are shown, representative of three independent experiments). B. MC38-OVAdim tumours (n=10, representative results of 1-3 experiments are shown). One-way ANOVA with posthoc Tukey's for multiple comparisons; *=p<0.05, **=p<0.01, ***=p<0.001 and ****=p<0.0001.

FIG. 4 shows representative flow cytometric contour plots showing live tumor infiltrating CD8+ PD-1+TIM3+ and CD8+ PD-1+TIM3+ TIGIT+ LAG3+ T cells (upper row) with corresponding quantification of IFN-γ production (n=10, mean±SD, experiment done once). One-way ANOVA with posthoc Tukey's for multiple comparisons; *=p<0.05, **=p<0.01, ***=p<0.001 and ****=p<0.0001.

FIG. 5 is a graphical representation showing the importance of Y319 to T cell function. (A) Individual growth curves of MC38-OVA^{dim} tumors in WT and CD226^{Y} mice (n=11 WT and n=12 CD226^{Y} mice, representative results of three experiments are shown). (B) Pie charts showing number of surviving (green) and dead mice (black) in WT and CD226^{Y} mice (n=30 WT and n=31 CD226^{Y} mice, cumulative results of three experiments are shown). (C) Corresponding Kaplan-Meier survival curves for experiment shown in (A). (D) Individual tumor growth curves of MC38-OVA^{hi} tumors in WT and CD226^{Y} mice (n=17 (Y)−21 (WT), one representative experiment of two is shown). (E) Pie charts showing number of surviving (green) and dead mice (black) in WT and CD226^{Y} mice. (n=57 WT and n=52 CD226^{Y} mice, cumulative results of two experiments are shown). (F) Corresponding Kaplan-Meier survival curves. (G) Kaplan-Meier survival curves of Vk12598 multiple myeloma bearing WT and CD226^{Y} mice (n=10 per group; experiment done once). (H) Flow cytometric quantification of CD226 MFI in resting splenic CD8+ T cells from CD226^{KO} WT or CD226^{Y} mice (n=6, mean±SD). (I) Representative flow cytometric histograms showing CD226 expression on CD8⁺ TILs from MC38-OVA^{dim} tumors. (J) Corresponding bar graphs showing mean frequency of indicated CD226 subsets in WT and CD226Y CD8⁺ T cells (left) and frequency of CD8⁺ CD226^{hi} T cells in individual mice (right, n=10, mean±SD, representative results of two experiments are shown). (K) Flow cytometric quantification of CD226^{neg} (left) and CD226^{dim} (right) CD8⁺ T cells isolated from MC38-OVA^{dim} tumors of WT or CD226^{Y} mice (n=10 per group, mean±SD, representative results of two experiments are shown). (L) Flow cytometric quantification of IFN-γ⁺ CD8⁺ TILs (n=18, mean±SD, cumulative results of two experiments are shown). (M) Representative flow cytometric contour plots showing live tumor-infiltrating CD8⁺ OVA-Tetramer⁺ T cells (left) and quantification of CD226^{hi} T cells in CD8⁺Tetramer^{neg} and CD8⁺ OVA-Tetramer⁺ T cells in WT and CD226^{Y} mice (right, n=10, mean±SD, representative results of two experiments are shown). (N) Flow cytometric quantification of IFN-γ⁺ in CD8⁺ OVA-Tetramer⁺ TILs (n=10, mean±SD, representative results of one experiment). (O) Quantification of IFN-γ and TNF-α in tumor tissue lysates determined by CBA (n=10, mean±SD, representative results of two experiments are shown). Statistics: Fisher's exact test (B, E), Log-rank (Mantel-Cox) test for survival curves (C, F), One-way ANOVA with posthoc Tukey's for multiple comparisons (H, J), Student's t-test (I, K, L); *p<0.05, **p<0.01, and ****p<0.0001.

FIG. 6 provides results showing that loss of CD226 in tumour infiltrating CD8+ T cells is mediated by CD155. (A) Correlation of the frequency of CD8⁺CD226^{neg} TILs with B16F10 tumor weights in mg (n=10, representative results of two experiments are shown). (B) Correlation of the frequency of CD8⁺CD226^{neg} TILs with MC3-OVA^{dim} tumor weights in mg (n=21, cumulative results of two experiments are shown). (C) Representative flow cytometric histogram showing CD226 expression in WT splenic CD8⁺ T cells left untreated or stimulated with plate-bound anti-CD3 in presence or absence of plate-bound mouse CD155-Fc and in presence or absence of α-CD155 blocking antibodies (top) and corresponding quantification (bottom). (n=6, mean±SD, representative results from two experiments are shown). (D) CD226 internalisation assay: representative fluorescent ImageStream pictures showing CD226 surface (red) and CD226 intracellular (yellow) staining of WT (top) or CD226^{Y} (bottom) CD8⁺ T cells treated with control IgG (IgG) or CD155-Fc (left) and corresponding quantification of intracellular CD226 MFI (right, n=100 cells, mean±SEM, representative results of two experiments are shown). (E) Experimental layout to assess the impact of tumor and host cell CD155 on CD226 expression in TILs (left). WT and CD155^{KO} mice were injected with B16F10^{Ctrl} or B16F10^{CD155KO} cells and 14 days after inoculation CD226 expression on CD8⁺ TILs was assessed (right, n=7 WT→WT, n=9 WT→KO, n=10 KO→WT and n=8 KO→KO, mean±SD, representative results of three experiments are shown). (F) Experimental layout for assessing the impact of CD226^{Y} in CD155 mediated CD226 downregulation (left). WT and CD226^{Y} mice were injected with B16F10^{Ctrl} or B16F10^{CD155KO} cells and 14 days after inoculation CD226 expression on CD8⁺ TILs was assessed (right, n=5 WT→WT, n=8 WT→CD226^{Y}, n=9 KO→WT and n=6 KO→CD226^{Y}, mean±SD, representative results of two experiments are shown). (G) Same experiment as in (F). WT and CD226^{Y} mice were injected with B16F10^{Ctrl} or B16F10^{OD155KO} cells and 14 days after inoculation CD226 expression on CD8⁺ TILs was assessed for CD226^{dim} (left) and CD226^{high} (right) (n=5 WT→WT, n=8 WT→CD226^{Y}, n=9 KO→WT and n=6 KO→CD226^{Y}, mean±SD, representative results of two experiments are shown). Statistics: Pearsons correlation with linear regression (A and B), One-way ANOVA with posthoc Tukey's for multiple comparisons (C, D, E, F and G); *p<0.05, **p<0.01, ***p<0.001 and ****p<0.0001.

FIG. 7 is a graphical and schematic representation of the methodology and results of adoptive transfer experiments. (A) Adoptive transfer of CD226⁺ (DNAM-1⁺) and CD226⁻ (DNAM-1⁻) CD8⁺ T cell into mice harboring B16F10 melanomas. On day 9, 5×10⁵ DNAM-1⁺ or DNAM-1⁻ gp100-specific CD8⁺ T cells were transferred intravenously in conjunction with a single injection of an adenoviral vaccine encoding for gp100. On day 12, 14 and 16 mice received intratumor polyI:C/CpG. Results are presented using a Kaplan-Meier-Curve (n=14 mice each group, pooled data from 2 independent experiments). ****=p<0.0001, Log-Rank Test. (B) Experimental protocol for ACT immunotherapy treating HCmel12^{hgp100} bearing WT mice with adoptive transfer of WT., CD226^{KO}. or CD226^{Y}.Pmel-1 T cells (Cy=cyclophosphamide). (C-E) Waterfall plots showing percentage of change in HCmel12^{hgp100} tumor area on day 14 after ACT therapy relative to pre-treatment (PD=progressive disease, PR=partial response and CR=complete response) for (C) WT.Pmel-1 (n=46), (C) CD226^{KO}.Pmel-1 (n=34) and (E) CD226^{Y}.Pmel-1 T cells (n=42). (F) Corresponding Pie charts showing the number of surviving (green) and dead mice (black) treated with indicated Pmel-1 T cells (cumulative results of three experiments are shown). (G) Kaplan-Meier survival curves for HCmel12^{hgp100} bearing WT mice treated with WT., CD226^{KO}. or CD226^{Y}.Pmel-1 T cells (n=46 WT.Pmel-1, n=34 CD226^{KO}.Pmel-1 and n=42 CD226^{Y}.Pmel-1 T cells, cumulative results of three experiments are shown). (H) Flow cytometric analyses showing the frequency of tumor infiltrating CD8⁺CD90.1⁺CD226^{hi} WT. or CD226^{Y}.Pmel-1 T cells (n=6 WT. and n=8 CD226^{Y}.Pmel-1, mean±SD, experiment done once) (I) Flow cytometric analyses showing the frequencies of IFN-γ⁺ tumor infiltrating WT or CD226^{Y} CD8⁺CD90.1⁺ Pmel-1 T cells (n=10 (WT.Pmel-1)-13 (CD226^{Y}.Pmel-1), mean±SD, cumulative results of two experiments are shown). (J) Experimental protocol to assess the effect of retroviral overexpression of CD226 on the efficacy of ACT. WT.Pmel-1 T cells were isolated and retrovirally transduced with an empty vector (MOCK) or CD226 and adoptively transferred into HCmel12^{hgp100} bearing WT mice. (K) Corresponding waterfall plots showing the percentage of change in HCmel12^{hgp100} tumor area on day 14 after ACT relative to pre-treatment (PD=progressive disease, PR=partial response and CR=complete response) (n=11 MOCK.Pmel-1 and n=12 CD226.Pmel-1, experiment done once). Statistics: Fishers exact test for (F), unpaired two-tailed Student's t-test (H, I) Log-rank (Mantel-Cox) test for survival curves; *p<0.05, **p<0.01, ***p<0.001 and ****p<0.0001.

FIG. 8 presents the results of studies assessing the importance of CD226 (DNAM-1) for the efficacy of immune checkpoint blockade (ICB). (A) Mean tumor size in WT or CD226^{KO} (DNAM-1^{KO}) mice administered anti-PD-1 antibody. Groups of 5-6 C57BL/6 WT or DNAM-1^{KO} mice were injected s.c. with MC38 colon adenocarcinoma cells (1×10⁵ cells). Groups of mice received 250 μg cIg or anti-PD1 (RMP1-14) mAb on days 10, 12, 14, and 16, relative to tumor inoculation on day 0. Groups of WT mice received either cIg or anti-DNAM-1 mAb (250 μg i.p.) on days 9, 10, 14, 17, 20, and 24. Subcutaneous primary tumor growth was measured at indicated times using digital calipers and mean tumor sizes+SEM are represented. (B) Waterfall plots showing the percentage of change in MC38-OVA^{dim} tumor area on day 14 after anti-PD1 therapy relative to pre-treatment in WT, CD226^{KO} and CD226^{Y} mice (PD=progressive disease, PR=partial response and CR=complete response) (n=9 WT, n=9 CD226^{KO} and n=8 CD226^{Y}, representative of two experiments). (C) Corresponding Kaplan-Meier survival curves (n for anti-PD1 as in (B), for cIg n=8 WT, n=9 CD226^{KO} and n=8 CD226^{Y}, representative results of two experiments). (D) Mean tumour growth curves of B16F10 melanoma in WT or CD226^{Y} mice treated as indicated (n=10 (WT+cIg and WT+anti-PD-1/anti-CTLA-4); n=7 (CD226^{Y}+cIg); n=8 (CD226^{Y}+anti-PD-1/anti-CTLA-4, mean±SEM, representative results of two experiments are shown)). Statistics: Logrank (Mantel-Cox) test for survival curvesLog-rank (Mantel-Cox) test for survival curves; Two-way ANOVA with posthoc Tukey's for multiple comparisons for (D); *p<0.05, ***p<0.001 and ****p<0.0001.

FIG. 9 shows the CD226 (DNAM-1) expression profile on T cells. (A) Representative flow cytometric histograms showing CD226 expression on resting and activated CD8⁺ T cells isolated from healthy donor PBMCs over time (left) and corresponding quantification of CD226 MFI (right, n=4 donors, mean±SD, representative results of two independent experiments are shown). (B) Nanostring analyses showing relative expression of IFNG and GZMB in healthy donor PBMCs after indicated stimulation. (C) Representative flow cytometric histogram showing CD226 expression in CD8⁺ tumor infiltrating T cells isolated from HNSCC patients (left) and corresponding quantification (right, n=10, mean±SD). (D) Representative flow cytometric contour plot showing IFN-γ⁺ cells across CD226 subsets (left) and corresponding quantification (right; n=10). (E) Corresponding data showing TNF-α⁺ cells across CD226 subsets (left) and corresponding quantification (right; n=10). (F) Corresponding data showing Ki67⁺ cells across CD226 subsets (left) and corresponding quantification (right; n=10). (G) Kaplan-Meier survical curves showing the survival probability of HNSCC (left) and SKCM (right) patients with high CD226 (top quartile) and low CD226 (bottom quartile) gene expression. Statistics: One-way ANOVA with posthoc Tukey's for multiple comparisons (D-F); *p<0.05, **p<0.01, *** and p<0.001.

FIG. 10 provides the results of studies showing the involvement of CD155 in CD226 (DNAM-1) down regulation. (A) Representative flow cytometric histogram showing CD155 expression in indicated CHO cells. (B) Representative flow cytometric histograms showing CD226 expression in pre-activated CD8⁺ T cells from healthy donor PBMCs incubated with CHO-OKT3 or CHO-OKT3-CD155 cells after 3 h of co-culture. (C) Corresponding quantification of CD226 expression over time (cumulative results of n=3 healthy donor PBMCs). (D) Representative histogram showing CD155 expression in indicated CHO-OKT3 cells (left) and quantification of CD226 loss in CD8⁺ T cells incubated for 3 h with indicated cells from healthy donor PBMCs (right). (E) Flow cytometric analyses of CD226 expression in CD8⁺ T cells incubated with CHO-OKT3 or CHO-OKT3-CD155 cells with increasing amounts of anti-CD155 blocking antibodies (representative results of n=3 healthy donor PBMCs). (F) Assessment of melanoma samples for CD155 expression and infiltration with CD226⁺CD8⁺ T cells. (G) Corresponding quantification and ratios of CD226⁺CD8⁺ to total CD8⁺ T cells in absent/low (n=9) and high (n=15) CD155 expressing melanomas (n=24, mean±SD). Statistics: unpaired two-tailed Student's t-test (G); **p<0.01.

FIG. 11 represents the results of a study investigating the correlation between DNAM-1 expression in CD8+ T cells with response to cancer immunotherapy in melanoma patients. (A) Assessment of pre-ICB melanoma samples for infiltration with CD226⁺CD8⁺ T cells and correlation with response and survival. (B) Upper row: contingency table for high and low ratios of CD226⁺CD8⁺ to total CD8+T cells against response. Responders were defined as CR and SD/PR with PFS>12 months; non-responders were defined as PD and SD/PR with PFS<12 months. Lower row: Kaplan-Meier survival curves showing progression-free survival of melanoma patients treated with immunotherapy with high (>0.07; blue) and low (<0.07; red) ratios of CD226⁺CD8⁺/CD8⁺ T cells as determined by multiplex-IHF (n=31 patients). (C) Upper row: contingency table for high and low counts of CD8⁺ T cells against response. Response was defined as in (B). Lower row: Kaplan-Meier survival curves showing progression-free survival of melanoma patients treated with immunotherapy with high>289; blue) and low (<289; red) CD8⁺ T cell infiltration counts determined by multiplex-IHF (n=31 patients). Statistics: Fisher's exact test (B, C), Log-rank (Mantel-Cox) test for survival curves (B, C).

FIG. 12 represents the results of a study assessing whether the Ubiquitin ligase E3 Cbl-2 is involved in DNAM-1 ubiquitination and internalization. CD8⁺ T cells from the spleens of wild-type mice or mice harbouring a point mutation in the CBL-B gene resulting in abrogation of the ubiquitin ligase function (Cbl-b^{KI} mice) were assessed for DNAM-1 (CD226) surface expression following stimulation with CD3/CD28 beads or CD3/CD28/CD155-Fc beads for 16 h in IL-2 (50 IU/ml hIL-2) containing cRPMI media. Histograms are from live CD8⁺ T cells.

Some figures contain color representations or entities. Color illustrations are available from the Applicant upon request or from an appropriate Patent Office. A fee may be imposed if obtained from a Patent Office.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” as used herein refers to the usual error range for the respective value readily known to the skilled person in this technical field. Reference to “about” a value or parameter herein includes (and describes) embodiments that are directed to that value or parameter per se.

“Activation”, as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and detectable effector functions, including performance of regulatory or cytolytic effector functions.

The term “activated T cell” means a T cell that is currently undergoing cell division, has detectable effector functions, including cytokine production, performs regulatory or cytolytic effector functions, and/or has recently undergone the process of “activation”.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

The “amount” or “level” of a polypeptide or polynucleotide is a detectable level in a sample. These can be measured by methods known to one skilled in the art and also disclosed herein.

As used herein, “and/or” refers to and encompasses any and all possible combinations of one or more of the associated listed items, as well as the lack of combinations when interpreted in the alternative (or).

The term “antagonist” or “inhibitor” refers to a substance that prevents, blocks, inhibits, neutralizes, or reduces a biological activity or effect of another molecule, such as a receptor.

The term “antibody”, as used herein, means any antigen-binding molecule or molecular complex comprising at least one complementarity determining region (CDR) that binds specifically to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or VH) and a heavy chain constant region. The heavy chain constant region comprises three domains, CH1, CH2 and CH3. Each light chain comprises a light chain variable region (which may be abbreviated as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of an antibody of the invention (or antigen-binding portion thereof) may be identical to the human germline sequences, or may be naturally or artificially modified. An amino acid consensus sequence may be defined based on a side-by-side analysis of two or more CDRs.

An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins.

The term “antigen-binding domain” refers to a region or portion of an antigen-binding molecule that participates in antigen-binding.

The term “antigen-binding fragment” refers to a part of an antigen-binding molecule that participates in antigen-binding. These terms include any naturally occurring, enzymatically obtainable, synthetic, or genetically engineered polypeptide or glycoprotein that specifically binds an antigen to form a complex. For example, antigen-binding fragments of an antibody may be derived from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (v) VH-CH1-CH2-CH3, (vi) VH-CH2-CH3; (vii) VH-CL; (viii) VL-CH1; (ix) VL-CH2, (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)). A multispecific antigen-binding molecule will typically comprise at least two different variable domains, wherein each variable domain is capable of specifically binding to a separate antigen or to a different epitope on the same antigen. Any multispecific antigen-binding molecule format may be adapted for use in the context of an antigen-binding fragment of an antibody of the present disclosure using routine techniques available in the art.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include antibodies and their antigen-binding fragments. The term “antigen-binding molecule” includes antibodies and antigen-binding fragments of antibodies.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “biomarker” as used herein refers to an indicator, e.g., predictive, diagnostic, and/or prognostic, which can be detected in a sample. The biomarker may serve as an indicator of a particular phenotype characterized by certain, molecular, pathological, histological, and/or clinical features. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, glycolipid-based molecular markers and cells comprising any of the aforementioned.

The terms “cancer” and “cancerous” refer to or describe the physiological condition in subjects that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers.

“Chemotherapeutic agent” includes compounds useful in the treatment of cancer.

The term “Chimeric Antigen Receptor” or “CAR” refers a molecule, which when in an immune effector cell, provides the cell with specificity for a target cell, typically a cancer cell, and with intracellular signal generation. In some embodiments, a CAR comprises at least an extracellular antigen-binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, “corresponding amino acid residues” (or positions) and grammatical variations thereof refer to residues (or positions) that occur at aligned loci within the primary amino acid sequence of a protein. Related or variant polypeptides are aligned by any method known to those of skill in the art. Such methods typically maximize matches, and include methods such as using manual alignments and by using the numerous alignment programs available (for example, BLASTP) and others known to those of skill in the art. By aligning the sequences of polypeptides, one skilled in the art can identify corresponding residues, using conserved and identical amino acid residues as guides. For example, by aligning the sequences of the human DNAM-1 polypeptide set forth in SEQ ID NO:1 with the mouse DNAM-1 polypeptide set forth in SEQ ID NO:3, one of skill in the art can identify corresponding residues using conserved and identical amino acid residues as guides, e.g. Y322 of SEQ ID NO:1 corresponds to Y319 of SEQ ID NO:3. Thus, for example, reference to a DNAM-1 polypeptide comprising a modification of a tyrosine at a position corresponding to position 322 of SEQ ID NO:1 includes reference to any DNAM-1 polypeptide that, when aligned the DNAM-1 polypeptide set forth in SEQ ID NO:1, has a modification of a tyrosine that is at an amino acid position that corresponds to (i.e. aligns with) amino acid position 322 of SEQ ID NO:1.

As used herein, the terms “cytolytic activity” and “cytotoxic activity” are used interchangeably herein and refer to the ability of a cell, e.g., a CD8+ cell or an NK cell, to lyse target cells. Such activity can be measured using standard techniques, e.g., by radioactively labeling the target cells.

The term “detection” includes any means of detecting, including direct and indirect detection.

The terms DNAM-1 and CD226 are used interchangeably throughout.

The term “DNAM-1 polypeptide” or “CD226 polypeptide” as used herein refers to a polypeptide comprising an amino acid sequence corresponding to a naturally-occurring DNAM-1 polypeptide. This term encompasses, without limitation, precursor DNAM-1 polypeptides such as those set forth in SEQ ID NO:1 (human) and SEQ ID NO:3 (mouse) and mature DNAM-1 polypeptides (i.e. lacking the N-terminal signal sequence) such as that set forth in SEQ ID NO:2 (human) and SEQ ID NO:4 (mouse). The term “DNAM-1 polypeptide” also encompasses, without limitation, polypeptides having an amino acid sequence that shares at least 80%, 81%, 82%, 83%, 84%, 85%, 86%, 88%, 89%, 90%, 91%, 92%, 93%, 94%, 95%, 96%, 97%, 98%, or 99% sequence identity with the sequence set forth in SEQ ID NO: 1 or 2 (across the entire sequence of SEQ ID NO:1 or 2 or a sequence comprising at least 100, 150, 200, 250, or 300 amino acids of the sequence set forth in SEQ ID NO: 1 or 2). Exemplary DNAM-1 polypeptides also include modified DNAM-1 polypeptides. As used herein, a “modified DNAM-1 polypeptide” refers to a DNAM-1 polypeptide having an amino acid sequence that contains one or more amino acid substitutions, deletions and/or additions relative to a wild-type DNAM-1 polypeptide (e.g. a wild-type human DNAM-1 polypeptide, such as one set forth in SEQ ID NO:1 or 2), i.e. the modified DNAM-1 polypeptide is modified relative to a wild-type or reference polypeptide. Typically, the modified DNAM-1 polypeptide retains at least or about 80%, 81%, 82%, 83%, 84%, 85%, 86%, 88%, 89%, 90%, 91%, 92%, 93%, 94%, 95%, 96%, 97%, 98%, or 99% sequence identity to the wild-type or reference DNAM-1 polypeptide, e.g. a wild-type human DNAM polypeptide set forth in SEQ ID NO: 1 or 2. In some examples, the modified DNAM-1 polypeptide is a “modified human DNAM-1 polypeptide”, which is a modified DNAM-1 polypeptide having one or more modifications relative to a wild-type human DNAM-1 polypeptide, such as a wild-type human DNAM-1 polypeptide set forth in SEQ ID NO:1 or 2 or a functional fragment thereof. Typically, modified human DNAM-1 polypeptide comprises at least 85%, 86%, 88%, 89%, 90%, 91%, 92%, 93%, 94%, 95%, 96%, 97%, 98%, or 99% sequence identity with the sequence set forth in SEQ ID NO: 1 or 2 (across the entire sequence of SEQ ID NO:1 or 2 or a sequence comprising at least 100, 150, 200, 250, or 300 amino acids of the sequence set forth in SEQ ID NO: 1 or 2). Non-limiting examples of modified DNAM-1 polypeptides include those comprising a modification of the tyrosine at an amino acid position corresponding to position 322 of SEQ ID NO:1, such as a Y322F modification; those comprising a modification of the serine at an amino acid position corresponding to position 329 of SEQ ID NO:1, such as a S329A modification; and those comprising a modification of the tyrosine at an amino acid position corresponding to position 322 and a modification of the serine at an amino acid position corresponding to position 329 of SEQ ID NO:1, such as a Y322F and a S329A modification; a modification in an AP-2 motif; a modification in the Cbl-b motif; a modification of the lysine at the position corresponding to position 295 and/or 333 of SEQ ID NO:1; and/or a modification of the glutamic acid at the position corresponding to position 282, the leucine at the position corresponding to position 286, and/or the phenylalanine at the position corresponding to position 287 of SEQ ID NO:1. In further examples, a modified DNAM-1 polypeptide of the present disclosure is one that lacks all or a portion of the IgG1 domain of a wild-type DNAM-1, all or a portion of the IgG2 domain of a wild-type DNAM-1, all or a portion of the IgG1 and IgG2 domains of a wild-type DNAM-1; and/or all or a portion of the intracellular domain of a wild-type DNAM-1. As would be appreciated, for the purposes of the present disclosure, the DNAM-1 polypeptide retains the ability of the wild-type DNAM-1 polypeptide to promote or facilitate T cell function, and in particular anti-tumor activity of the T cell in which the DNAM-1 polypeptide is expressed. Accordingly, in a particular embodiment, the activity of a DNAM-1 polypeptide is assessed in the context of its expression on a T cell, whereby the anti-tumor activity of the T cell expressing the DNAM-1 polypeptide is assessed to determine the activity of the DNAM-1 polypeptide. As used herein, a “DNAM-1 polynucleotide” refers to a polynucleotide that encodes a DNAM-1 polypeptide.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the cancer or tumor. In the case of an infection, an effective amount of the drug may have the effect in reducing pathogen (bacterium, virus, etc.) titers in the circulation or tissue; reducing the number of pathogen infected cells; inhibiting (i.e., slow to some extent or desirably stop) pathogen infection of organs; inhibit (i.e., slow to some extent and desirably stop) pathogen growth; and/or relieving to some extent one or more of the symptoms associated with the infection. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Enhancing T cell function” or “enhancing the function of a T cell” or grammatical variations thereof means to induce, cause or stimulate a T cell to have a sustained or amplified biological function, or renew or reactivate exhausted or inactive T cells. Examples of enhanced T cell function include any one or more of: increased secretion of IFN-γ, increased secretion of TNF-α, increased secretion of IL-2 from CD8+ T cells, increased proliferation, increased antigen responsiveness (e.g., viral, pathogen, or tumor clearance) relative to such levels before the intervention (e.g. before engineering a T cell to express recombinant DNAM-1). In some embodiments, the level of enhancement is as least 50%, alternatively 60%, 70%, 80%, 90%, 100%, 120%, 150%, or 200%. The manner of measuring this enhancement is known to one of ordinary skill in the art.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The terms “level of expression” or “expression level” in general are used interchangeably. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (e.g., transfer and ribosomal RNAs).

“Increased expression,” “increased expression levels,” or “increased levels” refers to an increased or elevated expression or level of a gene or protein in a sample (e.g., in or on a cell, tissue or organ) relative to a control sample. Expression or levels can be increased by at least or about 10%, 20%, 30%, 40%, 50%, 60%, 70%, 80%, 90%, 100%, 150%, 200%, 250%, 300%, 350%, 400% or more compared to a control.

“Decreased expression”, “decreased expression levels”, or “decreased levels” refers to a decreased or reduced expression or level of a gene or protein in a sample (e.g. in or on a cell, tissue or organ) relative to a control sample. Expression or levels can be decreased by at least or about 10%, 20%, 30%, 40%, 50%, 60%, 70%, 80%, 90% or more compared to a control.

The term “immune response” refers to any detectable response to a particular substance (such as an antigen or immunogen) by the immune system of a host mammal, such as innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids).

The term “intracellular signalling domain” refers to an intracellular region of a polypeptide that generates a signal that promotes an effector function in a cell (e.g. in the case of T cells, cytolytic activity or helper activity including the secretion of cytokines). In the context of an chimeric antigen receptor, an intracellular signaling domain comprises at least activating signaling domain (also referred to as a primary intracellular signaling domains), such as one comprising an immunoreceptor tyrosine-based activation motifs ITAM, e.g. those of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12). In some embodiments, the intracellular signaling domain also includes one or more costimulatory signaling domains, which include those derived from molecules responsible for costimulatory signals, or antigen independent stimulation (e.g. a CD28, 4-1BB or ICOS signaling domain). Reference to a “heterologous intracellular signaling domain” means an intracellular signaling domain that is not normally present in the polypeptide in question (e.g. a DNAM-1 polypeptide), i.e. is not normally present in the polypeptide in its natural state.

The term “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

The term “lymphocytes” as used herein refers to cells of the immune system which are a type of white blood cell. Lymphocytes include, but are not limited to, T-cells (cytotoxic and helper T-cells), B-cells and natural killer cells (NK cells).

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of enhanced T cell function, such as a subject with cancer or an infection

The term “pharmaceutical composition” or “pharmaceutical formulation” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., feces), tears, sweat, sebum, nipple aspirate, ductal lavage, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises T cells.

A “reference sample”, “reference cell”, “reference tissue”, “reference level”, “control sample”, “control cell”, “control tissue”, or “control level” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual, but at different time-points, e.g. before and after therapy. In another embodiment, a reference sample, reference cell, reference tissue, control sample, control cell, or control tissue is obtained from a healthy individual who is not the subject or individual being assessed. In particular examples, a reference sample, reference cell, reference tissue, control sample, control cell, or control tissue is or comprises a T-cell with normal or effective immune function, a T-cell with impaired or ineffective immune function, T-cells from a subject that is responsive or sensitive to therapy or T-cells from a subject that is non-responsive or resistant to therapy. In particular embodiments, the T-cells are CD8⁺ T-cells. In further examples, a reference level or control level is a level that is indicative of, or represents, a particular phenotype, such as a T-cell with normal or effective immune function, a T-cell with impaired or ineffective immune function, T-cells from a subject that is responsive or sensitive to therapy or T-cells from a subject that is non-responsive or resistant to therapy. In still further embodiments, the reference level or control level represents a “cut-off” above or below which is indicative of, or represents, a particular phenotype, such as a T-cell with normal or effective immune function, a T-cell with impaired or ineffective immune function, T-cells from a subject that is responsive or sensitive to therapy or T-cells from a subject that is non-responsive or resistant to therapy. In particular embodiments, the T-cells are CD8⁺ T-cells.

The term “sequence identity” as used herein refers to the extent that sequences are identical on a nucleotide-by-nucleotide basis or an amino acid-by-amino acid basis over a window of comparison (e.g. over 10, 15, 20, 30, 40, 50, 60, 70, 80, 90, 100, 120, 140, 160, 180, 200 or more nucleotides or amino acids residues). Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50.degree. C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual may be successfully “treated” if one or more symptoms associated with a cancer are mitigated or eliminated, including, but are not limited to, reducing the proliferation of (or destroying) cancerous cells, decreasing symptoms resulting from the cancer, increasing the quality of life of those suffering from the cancer, decreasing the dose of other medications or therapies required to treat the cancer, and/or prolonging survival of individuals. In the context of treatment of an infection, an individual may be successfully “treated” if one or more symptoms associated with infection are mitigated or eliminated, including, but are not limited to, reducing the number of infectious microorganisms in the subject, reducing symptoms resulting from the infection, and/or prolonging survival of individuals.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” “hyperproliferative disorder” and “tumor” are not mutually exclusive as referred to herein.

TABLE 1
Description of the sequences

| SEQ ID NO | Description |
|---|---|
| 1 | Precursor wild-type human DNAM-1 polypeptide |
| 2 | Mature wild-type human DNAM-1 polypeptide |
| 3 | Precursor wild-type mouse DNAM-1 polypeptide |
| 4 | Mature wild-type mouse DNAM-1 polypeptide |
| 5 | Precursor human DNAM-1 Y322 |
| 6 | Mature human DNAM-1 Y322F |
| 7 | Precursor human DNAM-1 lacking the cytoplasmic domain |
| 8 | Mature human DNAM-1 lacking the cytoplasmic domain |
| 9 | Human DNAM-1 extracellular domain |
| 10 | Precursor wild-type human DNAM-1 polynucleotide |
| 11 | Mouse DNAM-1 No IgG1 polynucleotide |
| 12 | Mouse DNAM-1 No IgG1 polypeptide |
| 13 | Mouse DNAM-1 No IgG1+ IgG2 polynucleotide |
| 14 | Mouse DNAM-1 No IgG1+ IgG2 polypeptide |
| 15 | Mouse DNAM-1 No intracellular polynucleotide |
| 16 | Mouse DNAM-1 No intracellular polypeptide |
| 17 | Mouse DNAM-1 S326A polynucleotide |
| 18 | Mouse DNAM-1 S326A polypeptide |
| 19 | Mouse DNAM-1 Y319A/S326A polynucleotide |
| 20 | Mouse DNAM-1 Y319A/S326A polypeptide |
| 21 | Precursor human DNAM-1 Y325A/F328A |
| 22 | Mature human DNAM-1 Y325A/F328A |
| 23 | Precursor human DNAM-1 E282A/L286A/F287A |
| 24 | Mature human DNAM-1 E282A/L286A/F287A |
| 25 | Precursor human DNAM-1 K295A |
| 26 | Mature human DNAM-1 K295A |
| 27 | Precursor human DNAM-1 K333A |
| 28 | Mature human DNAM-1 K333A |
| 29 | Precursor human DNAM-1 K295A/K333A |
| 30 | Mature human DNAM-1 K295A/K333A |

Each embodiment described herein is to be applied mutatis mutandis to each and every embodiment unless specifically stated otherwise.

2. T Cells with Enhanced Function

The present disclosure is based in part on the determination that DNAM-1 (also referred to as CD226) is essential for immune function of T cells in the tumor environment. Accordingly, provided are T cells that express DNAM-1 on the cell surface, such as recombinant DNAM-1, including modified DNAM-1. Also in accordance with the present disclosure, methods are provided that take advantage of DNAM-1 expression on the surface of a T cell to enhance T cell (e.g., CD8+ T cell) function, including increasing T cell activation. The T cells and methods of the present disclosure are thus particularly useful in the treatment of cancer as part of adoptive cell transfer immunotherapy. The T cells and methods of the present disclosure are also useful in the treatment of infection. For example, the T cells of the disclosure can be adoptively transferred to a subject with a chronic infection, wherein endogenous T cells may be exhausted. The T cells of the present disclosure that express DNAM-1 on the surface can exhibit, for example enhanced activation (as measured by, for example, IFN-γ, IL-2 or TNF expression), enhanced proliferation (as measured by, for example, Ki67 expression), enhanced cytolytic activity, and/or enhanced anti-tumor activity compared to T cells that do not expressed DNAM-1 on the surface. Any one or more T cell immune functions can be increased by at least or about 10%, 20%, 30%, 40%, 50%, 60%, 70%, 80%, 90% 100%, 110%, 120%, 130%, 140%, 150%, 200%, 300%, 400%, 500% or more compared to a T cells that do not express DNAM-1 on the surface.

2.1 DNAM-1

DNAM-1 was first described as an adhesion molecule involved in the cytotoxic properties of T cells (Shibuya et al., 1996, Immunity 4(6):573-581). It is mainly expressed by CD4 and CD8 T cells, NK cells, platelets and monocytes, with its ligand being CD155 (poliovirus receptor) and CD112 (nectin-2), which are themselves expressed on a broad range of cells, including, APCs, transformed cells and virus-infected cells. DNAM-1 appears to be involved in multiple cellular processes: it is thought to be important in immune cell extravasation, relevant for the stability of the immunological synapse, an important NK cell activating receptor and a co-receptor for CD4 T cells. Thus, DNAM-1 deficient mice are more protected against GVHD and are more susceptible to carcinogen-induced tumorigenesis (see e.g. Nabekura et al. 2010, Proc Natl Acad Sci USA. 107(43):18593-18598; Iguchi-Manaka et al., 2008, J Exp Med. 205(13): 2959-2964).

The precursor human DNAM-1 is a 336-amino acid polypeptide (set forth in SEQ ID NO:1), which is processed by removal of the 18 amino acid N-terminal signal peptide to produce a 318-amino acid mature DNAM-1 polypeptide (set forth in SEQ ID NO:2). In addition to the signal peptide, the precursor DNAM-1 comprises a 230-amino acid extracellular domain (amino acid positions 19 to 248 of SEQ ID NO:1), a 28 amino acid transmembrane domain (amino acid positions 249 to 276 of SEQ ID NO:1) and a 60 amino acid cytoplasmic domain (amino acid positions 277 to 336 of SEQ ID NO:1). Although DNAM-1 is part of the Ig superfamily, it is unique in its structure. For example, the cytoplasmic domain shares little or no homology with other Ig superfamily members DNAM-1.

DNAM-1 has two extracellular domains important for its binding to CD155: the IgG1 domain (corresponding to approximately amino acid residues 19-126 of SEQ ID NO:1) and the IgG2 domain (corresponding to approximately amino acid residues 135-239 of SEQ ID NO:1). DNAM-1 also contains an immunoglobulin tyrosine tail (ITT) motif (YVNY) for intracellular signalling (Zhang et al. 2015, J Exp Med. 212(12):2165-2182). DNAM-1 has three phosphorylation sites: Y322 (which is in the ITT motif), Y325 (which might be implicated in regulating DNAM-1 expression) and S329, each of which are associated with several functions. While it has been shown that signalling through Y322 is absolutely required for the activation of NK cells, the role of DNAM-1 signalling in T cells has been unclear.

Exemplary wild-type DNAM-1 polypeptides include wild-type precursor DNAM-1 polypeptides (including the human wild-type precursor DNAM-1 polypeptide set forth in SEQ ID NO:1 and the mouse wild-type precursor DNAM-1 polypeptide set forth in SEQ ID NO:3) and wild-type mature DNAM-1 polypeptides (including the human wild-type mature DNAM-1 polypeptide set forth in SEQ ID NO:2 and the mouse wild-type mature DNAM-1 polypeptide set forth in SEQ ID NO:4).

A representative precursor wild-type human DNAM-1 polypeptide has the following sequence (N-terminal signal peptide is in bold; underlined residues are Y322, Y325 and S329):

(SEQ ID NO: 1)
MDYPTLLLALLHVYRALCEEVLWHTSVPFAENMSLECVYPSMGILTQVEW
FKIGTQQDSIAIFSPTHGMVIRKPYAERVYFLNSTMASNNMTLFFRNASE
DDVGYYSCSLYTYPQGTWQKVIQVVQSDSFEAAVPSNSHIVSEPGKNVTL
TCQPQMTWPVQAVRWEKIQPRQIDLLTYCNLVHGRNFTSKFPRQIVSNCS
HGRWSVIVIPDVTVSDSGLYRCYLQASAGENETFVMRLTVAEGKTDNQYT
LFVAGGTVLLLLFVISITTIIVIFLNRRRRRERRDLFTESWDTQKAPNNY
RSPISTSQPTNQSMDDTREDIYVNYPTFSRRPKTRV

A representative mature wild-type human DNAM-1 polypeptide has the following sequence:

(SEQ ID NO: 2)
EEVLWHTSVPFAENMSLECVYPSMGILTQVEWFKIGTQQ
DSIAIFSPTHGMVIRKPYAERVYFLNSTMASNNMTLFFR
NASEDDVGYYSCSLYTYPQGTWQKVIQVVQSDSFEAAVP
SNSHIVSEPGKNVTLTCQPQMTWPVQAVRWEKIQPRQID
LLTYCNLVHGRNFTSKFPRQIVSNCSHGRWSVIVIPDVT
VSDSGLYRCYLQASAGENETFVMRLTVAEGKTDNQYTLF
VAGGTVLLLLFVISITTIIVIFLNRRRRRERRDLFTESW
DTQKAPNNYRSPISTSQPTNQSMDDTREDIYVNYPTFSR
RPKTRV

A representative precursor wild-type mouse DNAM-1 polypeptide has the following sequence (N-terminal signal peptide is in bold; underlined residues are Y319, Y322 and S326):

(SEQ ID NO: 3)
MAYVTWLLAILHVHKALCEETLWDTTVRLSETMTLECVYPLTHNLTQVEW
TKNTGTKTVSIAVYNPNHNMHIESNYLHRVHFLNSTVGFRNMSLSFYNAS
EADIGIYSCLFHAFPNGPWEKKIKVVWSDSFEIAAPSDSYLSAEPGQDVT
LTCQLPRTWPVQQVIWEKVQPHQVDILASCNLSQETRYTSKYLRQTRSNC
SQGSMKSILIIPNAMAADSGLYRCRSEAITGKNKSFVIRLIITDGGTNKH
FILPIVGGLVSLLLVILIIIIFILYNRKRRRQVRIPLKEPRDKQSKVATN
CRSPTSPIQSTDDEKEDIYVNYPTFSRRPKPRL

A representative mature wild-type mouse DNAM-1 polypeptide has the following sequence:

(SEQ ID NO: 4)
EETLWDTTVRLSETMTLECVYPLTHNLTQVEWTKNTGTKTVSIAVYNPNH
NMHIESNYLHRVHFLNSTVGFRNMSLSFYNASEADIGIYSCLFHAFPNGP
WEKKIKVVWSDSFEIAAPSDSYLSAEPGQDVTLTCQLPRTWPVQQVIWEK
VQPHQVDILASCNLSQETRYTSKYLRQTRSNCSQGSMKSILIIPNAMAAD
SGLYRCRSEAITGKNKSFVIRLIITDGGTNKHFILPIVGGLVSLLLVILI
IIIFILYNRKRRRQVRIPLKEPRDKQSKVATNCRSPTSPIQSTDDEKEDI
YVNYPTFSRRPKPRL

In some embodiments, DNAM-1 polypeptides of the present disclosure can exhibit increased retention on the surface of a T cell compared to a wild-type DNAM-1 polypeptide. Such DNAM-1 polypeptides comprise one or more modifications relative to a wild-type DNAM-1 polypeptide (i.e. they are modified DNAM-1 polypeptides), wherein the one or more modifications impart increased cell surface retention (or decreased internalization) of the DNAM-1 polypeptide when expressed on the surface of a T cell (e.g. a CD8+ T cell). Typically, the modified DNAM-1 polypeptides comprise one or more amino acid modifications (e.g. deletions, insertions or substitutions) relative to a wild-type DNAM-1 polypeptide, such that the amino acid sequence of the modified DNAM-1 polypeptide is less than 100% identical to a wild-type DNAM-1 polypeptide, such as a wild-type human DNAM-1 polypeptide set forth in SEQ ID NO:1 or 2. In some examples, the modified DNAM-1 polypeptide retains at least or about 85%, 86%, 87%, 88%, 89%, 90%, 91%, 92%, 93%, 94%, 95%, 96%, 98% or 99% sequence identity to a wild-type DNAM-1 polypeptide, such as a wild-type human DNAM-1 polypeptide set forth in SEQ ID NO:1 or 2. For example, modified DNAM-1 polypeptides of the present disclosure can comprise a sequence set forth in SEQ ID NO:1 or 2 or a sequence having at least or about 85%, 86%, 87%, 88%, 89%, 90%, 91%, 92%, 93%, 94%, 95%, 96%, 98% or 99% sequence identity, but further comprise at least one amino acid modification described below that impart increased cell surface retention (or decreased internalization) of the DNAM-1 polypeptide when expressed on the surface of a T cell (e.g. a CD8+ T cell). In particular examples, therefore, the modified DNAM-1 polypeptides comprise at least one of the modifications described below and a sequence having at most 85%, 86%, 87%, 88%, 89%, 90%, 91%, 92%, 93%, 94%, 95%, 96% or 98% sequence identity to a wild-type DNAM-1 polypeptide, e.g. a wild-type DNAM-1 polypeptide sequence set forth in SEQ ID NO:1 or 2.

Exemplary of the modified DNAM-1 polypeptides provided herein are those comprising a modification of the tyrosine at the amino acid position corresponding to position 322 of SEQ ID NO:1 have increased retention on the surface of a T cell compared to wild-type DNAM-1 polypeptides and facilitate enhanced T cell function. Thus, exemplary DNAM-1 polypeptides for expression in T cells of the present disclosure also include DNAM-1 comprising a modification of the tyrosine at the amino acid position corresponding to position 322 of SEQ ID NO:1. Such DNAM-1 polypeptides can exhibit reduced (including abolished) signalling through phosphorylation of the residue at position 322 compared to a wild-type DNAM-1 polypeptide. The modification of the tyrosine at the amino acid position corresponding to position 322 of SEQ ID NO:1 can be, for example, an amino acid deletion or any amino acid substitution, such as a substitution with alanine, asparagine, aspartic acid, cysteine, glutamine, glutamic acid, glycine, histidine, isoleucine, leucine, lysine, methionine, phenylalanine, proline, serine, threonine, tryptophan or valine. In a particular example, the amino acid substitution is a substitution with alanine, asparagine, aspartic acid, cysteine, glutamine, glutamic acid, glycine, histidine, isoleucine, leucine, lysine, methionine, phenylalanine, proline, tryptophan or valine. In one embodiment, the substitution is with phenylalanine (e.g. Y322F; such as set forth in SEQ ID NOs:5 and 6) or alanine (e.g. Y322A).

Precursor human DNAM-1 Y322F (N-terminal signal peptide is in bold; underlined residue is F322):

(SEQ ID NO: 5)
MDYPTLLLALLHVYRALCEEVLWHTSVPFAENMSLECVYPSMGILTQVEW
FKIGTQQDSIAIFSPTHGMVIRKPYAERVYFLNSTMASNNMTLFFRNASE
DDVGYYSCSLYTYPQGTWQKVIQVVQSDSFEAAVPSNSHIVSEPGKNVTL
TCQPQMTWPVQAVRWEKIQPRQIDLLTYCNLVHGRNFTSKFPRQIVSNCS
HGRWSVIVIPDVTVSDSGLYRCYLQASAGENETFVMRLTVAEGKTDNQYT
LFVAGGTVLLLLFVISITTIIVIFLNRRRRRERRDLFTESWDTQKAPNNY
RSPISTSQPTNQSMDDTREDIFVNYPTFSRRPKTRV

Mature human DNAM-1 Y322F:

(SEQ ID NO: 6)
EEVLWHTSVPFAENMSLECVYPSMGILTQVEWFKIGTQQDSIAIFSPTHG
MVIRKPYAERVYFLNSTMASNNMTLFFRNASEDDVGYYSCSLYTYPQGTW
QKVIQVVQSDSFEAAVPSNSHIVSEPGKNVTLTCQPQMTWPVQAVRWEKI
QPRQIDLLTYCNLVHGRNFTSKFPRQIVSNCSHGRWSVIVIPDVTVSDSG
LYRCYLQASAGENETFVMRLTVAEGKTDNQYTLFVAGGTVLLLLFVISIT
TIIVIFLNRRRRRERRDLFTESWDTQKAPNNYRSPISTSQPTNQSMDDTR
EDIFVNYPTFSRRPKTRV

In further examples, increased surface retention may be achieved by targeting amino acid residues or motifs involved in internalization such that internalization of DNAM-1 is inhibited or reduced. Signaling and function of receptors such as DNAM-1 can be regulated by removal of the receptor from the cell surface through endocytic internalization. This principle has been shown for receptor tyrosine kinases (e.g. EGFR), G-Protein coupled receptors and also for immune-related receptors e.g. (CD3, CD4, CTLA-4 etc.). A large number of different mechanism and endocytic pathways exist, but two of the more important pathways are clathrin-mediated-endocytosis (CME) and ubiquitination. CME is usually mediated through binding of the adaptor protein AP-2 to a motif in the cytoplasmic tail of the surface receptor. CME via AP-2 is frequently associated with receptor recycling and surface re-expression. Conversely, polyubiquitination of a receptor leads to internalization and subsequent degradation. Thus, provided herein are modified DNAM-1 polypeptides comprising one or more modifications that target (i.e. abolish) an AP-2 binding motif, an E3 ubiquitin ligase binding (Cbl-b) motif, and/or a ubiquitination site.

DNAM-1 has an AP-2 binding motif, YXXF, in its cytoplasmic tail at amino acid positions corresponding to positions 325-328 (residues YPTF) of the precursor DNAM-1 set forth in SEQ ID NO:1. Accordingly, other exemplary DNAM-1 polypeptides of the present disclosure include those in which the YXXF AP-2 motif has been modified, such that CME of DNAM-1 via AP-2 and is reduced or abolished, thereby increasing cell surface retention of DNAM-1. As would be appreciated, the AP-2 motif can be abolished by any of a number of modifications, including amino acid deletion of any one or more of residues Y, P, T, F at positions corresponding to 325-328, respectively, of SEQ ID NO:1; amino acid substitution of the tyrosine at the position corresponding to position 325 of SEQ ID NO:1, and/or the phenylalanine at the position corresponding to position 328 of SEQ ID NO:1; and/or amino acid insertion after any one of the positions corresponding to position 325, 326 or 327 of SEQ ID NO:1. Exemplary DNAM-1 polypeptides therefore include those having a modification of the tyrosine at the position corresponding to position 325 of SEQ ID NO:1, and/or the phenylalanine at the position corresponding to position 328 of SEQ ID NO:1. The modification can be any modification (e.g. deletion and/or substitution) that results in abolition of the YXXF motif. In particular examples, the modification is a substitution, such as a substitution of the tyrosine at position 325 with alanine, asparagine, aspartic acid, cysteine, glutamine, glutamic acid, glycine, histidine, isoleucine, leucine, lysine, methionine, phenylalanine, proline, serine, threonine, tryptophan or valine, and/or a substitution of the phenylalanine at position 328 with alanine, asparagine, aspartic acid, cysteine, glutamine, glutamic acid, glycine, histidine, isoleucine, leucine, lysine, methionine, tyrosine, proline, serine, threonine, tryptophan or valine. In a non-limiting example, the DNAM-1 polypeptide comprises substitutions of the tyrosine at position 325 with alanine and the phenylalanine at position 328 with alanine.

Precursor human DNAM-1 Y325A/F328A (N-terminal signal peptide is in bold; underlined residues are A325 and A328):

(SEQ ID NO: 21)
MDYPTLLLALLHVYRALCEEVLWHTSVPFAENMSLECVYPSMGILTQVEW
FKIGTQQDSIAIFSPTHGMVIRKPYAERVYFLNSTMASNNMTLFFRNASE
DDVGYYSCSLYTYPQGTWQKVIQVVQSDSFEAAVPSNSHIVSEPGKNVTL
TCQPQMTWPVQAVRWEKIQPRQIDLLTYCNLVHGRNFTSKFPRQIVSNCS
HGRWSVIVIPDVTVSDSGLYRCYLQASAGENETFVMRLTVAEGKTDNQYT
LFVAGGTVLLLLFVISITTIIVIFLNRRRRRERRDLFTESWDTQKAPNNY
RSPISTSOPTNOSMDDTREDIYVNAPTASRRPKTRV

Mature human DNAM-1 Y325A/F328A:

(SEQ ID NO: 22)
EEVLWHTSVPFAENMSLECVYPSMGILTQVEWFKIGT
QQDSIAIFSPTHGMVIRKPYAERVYFLNSTMASNNMTLFF
RNASEDDVGYYSCSLYTYPQGTWQKVIQVVQSDSFEAAVP
SNSHIVSEPGKNVTLTCQPQMTWPVQAVRWEKIQPRQIDL
LTYCNLVHGRNFTSKFPRQIVSNCSHGRWSVIVIPDVTVS
DSGLYRCYLQASAGENETFVMRLTVAEGKTDNQYTLFVAG
GTVLLLLFVISITTIIVIFLNRRRRRERRDLFTESWDTQK
APNNYRSPISTSQPTNQSMDDTREDIYVNAPTASRRPKTRV

Human DNAM-1 has a second AP-2 binding motif, EXXXLF, which would target alpha2/sigma2 subunits of AP-2 (a2/s2 hemicomplex). This motif is present in wild-type human DNAM-1 polypeptides at amino acid positions corresponding to positions 282-287 (residues ERRDLF) of SEQ ID NO:1. Accordingly, other exemplary DNAM-1 polypeptides of the present disclosure include those in which the EXXXLF AP-2 motif has been modified, such that CME of DNAM-1 via AP-2 and is reduced or abolished, thereby increasing cell surface retention of DNAM-1. As would be appreciated, the EXXXLF AP-2 motif can be abolished by any of a number of modifications, including amino acid deletion of any one or more of residues E, R, R, D, L or F at positions corresponding to 282-287, respectively, of SEQ ID NO:1; amino acid substitution of glutamic acid at position 282, the leucine at position 286 and/or the phenylalanine at position 287 of the human precursor DNAM-1 set forth in SEQ ID NO:1; and/or insertion of an amino acid residue after the residues at positions corresponding to 282-286, respectively. In particular examples, the modification is a substitution, such as a substitution of the glutamic acid at position 282 with alanine, asparagine, aspartic acid, cysteine, glutamine, tyrosine, glycine, histidine, isoleucine, leucine, lysine, methionine, phenylalanine, proline, serine, threonine, tryptophan or valine; a substitution of the leucine at position 286 with alanine, asparagine, aspartic acid, cysteine, glutamine, glutamic acid, glycine, histidine, isoleucine, phenylalanine, lysine, methionine, tyrosine, proline, serine, threonine, tryptophan or valine; and/or a substitution of the phenylalanine at position 287 with alanine, asparagine, aspartic acid, cysteine, glutamine, glutamic acid, glycine, histidine, isoleucine, leucine, lysine, methionine, tyrosine, proline, serine, threonine, tryptophan or valine. In a non-limiting example, the DNAM-1 polypeptide comprises substitutions of the glutamic acid at position 282 with alanine, the leucine at position 286 with alanine, and the phenylalanine at position 287 with alanine.

Precursor human DNAM-1 E282A/L286A/F287A (N-terminal signal peptide is in bold; underlined residues are A282, A286 and A287):

(SEQ ID NO: 23)
MDYPTLLLALLHVYRALCEEVLWHTSVPFAENMSLECVYPS
MGILTQVEWFKIGTQQDSIAIFSPTHGMVIRKPYAERVYFL
NSTMASNNMTLFFRNASEDDVGYYSCSLYTYPQGTWQKVIQ
VVQSDSFEAAVPSNSHIVSEPGKNVTLTCQPQMTWPVQAVR
WEKIQPRQIDLLTYCNLVHGRNFTSKFPRQIVSNCSHGRWS
VIVIPDVTVSDSGLYRCYLQASAGENETFVMRLTVAEGKTD
NQYTLFVAGGTVLLLLFVISITTIIVIFLNRRRRRARRDAA
TESWDTQKAPNNYRSPISTSQPTNQSMDDTREDIYVNAPTA
SRRPKTRV

Mature human DNAM-1 E282A/L286A/F287A:

(SEQ ID NO: 24)
EEVLWHTSVPFAENMSLECVYPSMGILTQVEWFKIGTQQD
SIAIFSPTHGMVIRKPYAERVYFLNSTMASNNMTLFFRNA
SEDDVGYYSCSLYTYPQGTWQKVIQVVQSDSFEAAVPSNS
HIVSEPGKNVTLTCQPQMTWPVQAVRWEKIQPRQIDLLTY
CNLVHGRNFTSKFPRQIVSNCSHGRWSVIVIPDVTVSDSG
LYRCYLQASAGENETFVMRLTVAEGKTDNQYTLFVAGGTV
LLLLFVISITTIIVIFLNRRRRRARRDAATESWDTQKAPN
NYRSPISTSQPTNQSMDDTREDIYVNAPTASRRPKTRV

Human DNAM-1 also has a binding motif ((D/N)XpY) for the E3 ubiquitin ligase Cbl-b at positions corresponding to 320-322 of the precursor DNAM-1 set forth in SEQ ID NO:1. As demonstrated herein, Cbl-b is involved in CD155-mediated DNAM-1 downregulation, wherein abrogation of Cbl-b function results in DNAM-1 cell surface retention. Accordingly, provided herein are modified DNAM-1 polypeptides that comprise one or modifications relative to a wild-type DNAM-1 polypeptide, wherein the modifications target the Cbl-b (D/N)XpY binding motif and/or the ubiquitination sites, such that the modified DNAM-1 polypeptides exhibit increased cell surface retention compared to a wild-type DNAM-1 polypeptide. Binding of Cbl-b requires phosphorylation of Y322, and the absence of this phosphorylation site may prevent internalization and degradation of DNAM-1 (consistent with the demonstration herein that targeting Y322 of human DNAM-1 results in increased cell surface retention of DNAM-1). In other examples, the DNAM-1 polypeptide comprises an amino acid insertion after any one or more of the aspartic acid at the position corresponding to position 320, or the amino acid residue at the position corresponding to position 321 of SEQ ID NO:1, so as to abolish the Cbl-b binding motif. In a further example, the DNAM-1 polypeptide comprises an amino acid deletion or substitution of the aspartic acid at the position corresponding to position 320 of SEQ ID NO:1 (e.g. substitution with an alanine, lysine, cysteine, glutamine, tyrosine, glycine, histidine, isoleucine, leucine, glutamic acid, methionine, phenylalanine, proline, serine, threonine, tryptophan or valine). Ubiquitination takes place on lysine (K) residues. Consequently, it is expected that modification of the lysine at position 295 and/or 333 (with numbering relative to SEQ ID NO:1) to abolish these ubiquitination sites also leads to decreased internalization and degradation of DNAM-1 and thus the retention of DNAM-1 surface expression. Further DNAM-1 polypeptides of the present disclosure that are suitable for expression in T cells therefore include those having a modification (e.g. amino acid deletion, insertion and/or substitution) of the lysine at the position corresponding to position 295 and/or the lysine at the position corresponding to position 333 of the human precursor DNAM-1 set forth in SEQ ID NO:1. In particular examples, the modification is a substitution, i.e. a substitution of the lysine at position 295 with alanine, asparagine, aspartic acid, cysteine, glutamine, tyrosine, glycine, histidine, isoleucine, leucine, glutamic acid, methionine, phenylalanine, proline, serine, threonine, tryptophan or valine; a substitution of the lysine at position 333 with alanine, asparagine, aspartic acid, cysteine, glutamine, glutamic acid, glycine, histidine, isoleucine, phenylalanine, leucine, methionine, tyrosine, proline, serine, threonine, tryptophan or valine. In non-limiting examples, the DNAM-1 polypeptide comprises substitution of the lysine at position 295 with alanine, and/or the lysine at position 333 with alanine.

Precursor human DNAM-1 K295A (N-terminal signal peptide is in bold; underlined residue is A295):

(SEQ ID NO: 25)
MDYPTLLLALLHVYRALCEEVLWHTSVPFAENMSLECV
YPSMGILTQVEWFKIGTQQDSIAIFSPTHGMVIRKPYA
ERVYFLNSTMASNNMTLFFRNASEDDVGYYSCSLYTYP
QGTWQKVIQVVQSDSFEAAVPSNSHIVSEPGKNVTLTC
QPQMTWPVQAVRWEKIQPRQIDLLTYCNLVHGRNFTSK
FPRQIVSNCSHGRWSVIVIPDVTVSDSGLYRCYLQASA
GENETFVMRLTVAEGKTDNQYTLFVAGGTVLLLLFVIS
ITTIIVIFLNRRRRRERRDLFTESWDTQAAPNNYRSPI
STSOPTNOSMDDTREDIYVNYPTFSRRPKTRV

Mature human DNAM-1 K295A:

(SEQ ID NO: 26)
EEVLWHTSVPFAENMSLECVYPSMGILTQVEWFKIGTQ
QDSIAIFSPTHGMVIRKPYAERVYFLNSTMASNNMTL
FFRNASEDDVGYYSCSLYTYPQGTWQKVIQVVQSDSF
EAAVPSNSHIVSEPGKNVTLTCQPQMTWPVQAVRWE
KIQPRQIDLLTYCNLVHGRNFTSKFPRQIVSNCSHGR
WSVIVIPDVTVSDSGLYRCYLQASAGENETFVMRLTVA
EGKTDNQYTLFVAGGTVLLLLFVISITTIIVIFLNRRR
RRERRDLFTESWDTQAAPNNYRSPISTSQPTNQSMDDT
REDIYVNYPTFSRRPKTRV

Precursor human DNAM-1 K333A (N-terminal signal peptide is in bold; underlined residue is A333):

(SEQ ID NO: 27)
MDYPTLLLALLHVYRALCEEVLWHTSVPFAENMSLECVY
PSMGILTQVEWFKIGTQQDSIAIFSPTHGMVIRKPYAE
RVYFLNSTMASNNMTLFFRNASEDDVGYYSCSLYTYPQ
GTWQKVIQVVQSDSFEAAVPSNSHIVSEPGKNVTLTCQ
PQMTWPVQAVRWEKIQPRQIDLLTYCNLVHGRNFTSKF
PRQIVSNCSHGRWSVIVIPDVTVSDSGLYRCYLQASA
GENETFVMRLTVAEGKTDNQYTLFVAGGTVLLLLFVI
SITTIIVIFLNRRRRRERRDLFTESWDTQKAPNNYR
SPISTSQPTNQSMDDTREDIYVNYPTFSRRPATRV

Mature human DNAM-1 K333A:

(SEQ ID NO: 28)
EEVLWHTSVPFAENMSLECVYPSMGILTQVEWFKIGTQ
QDSIAIFSPTHGMVIRKPYAERVYFLNSTMASNNMTL
FFRNASEDDVGYYSCSLYTYPQGTWQKVIQVVQSDSFE
AAVPSNSHIVSEPGKNVTLTCQPQMTWPVQAVRWEKI
QPRQIDLLTYCNLVHGRNFTSKFPRQIVSNCSHGRWS
VIVIPDVTVSDSGLYRCYLQASAGENETFVMRLTVAE
GKTDNQYTLFVAGGTVLLLLFVISITTIIVIFLNRRRR
RERRDLFTESWDTQKAPNNYRSPISTSQPTNQSMDDT
REDIYVNYPTFSRRPATRV

Precursor human DNAM-1 K295A/K333A (N-terminal signal peptide is in bold; underlined residue is A295 and A333):

(SEQ ID NO: 29)
MDYPTLLLALLHVYRALCEEVLWHTSVPFAENMSLEC
VYPSMGILTQVEWFKIGTQQDSIAIFSPTHGMVIRK
PYAERVYFLNSTMASNNMTLFFRNASEDDVGYYSCSL
YTYPQGTWQKVIQVVQSDSFEAAVPSNSHIVSEPGKN
VTLTCQPQMTWPVQAVRWEKIQPRQIDLLTYCNLVHG
RNFTSKFPRQIVSNCSHGRWSVIVIPDVTVSDSGLYR
CYLQASAGENETFVMRLTVAEGKTDNQYTLFVAGGTVLL
LLFVISITTIIVIFLNRRRRRERRDLFTESWDTQAAPN
NYRSPISTSQPTNQSMDDTREDIYVNYPTFSRRPATRV

Mature human DNAM-1 K295A/K333A:

(SEQ ID NO: 30)
EEVLWHTSVPFAENMSLECVYPSMGILTQVEWFKIGT
QQDSIAIFSPTHGMVIRKPYAERVYFLNSTMASNNMTL
FFRNASEDDVGYYSCSLYTYPQGTWQKVIQVVQSDSF
EAAVPSNSHIVSEPGKNVTLTCQPQMTWPVQAVRWE
KIQPRQIDLLTYCNLVHGRNFTSKFPRQIVSNCSHGRW
SVIVIPDVTVSDSGLYRCYLQASAGENETFVMRLTVA
EGKTDNQYTLFVAGGTVLLLLFVISITTIIVIFLNRRR
RRERRDLFTESWDTQAAPNNYRSPISTSQPTNQSMDDT
REDIYVNYPTFSRRPATRV

Other exemplary DNAM-1 polypeptides suitable for expression in T cells include those comprising a modification of the serine at the amino acid position corresponding to position 329 of SEQ ID NO:1. Such DNAM-1 polypeptides can exhibit reduced (including abolished) signalling through phosphorylation of the residue at position 329 compared to a wild-type DNAM-1 polypeptide. The modification of the serine at the amino acid position corresponding to position 329 of SEQ ID NO:1 can be, for example, an amino acid deletion or any amino acid substitution, such as a substitution with alanine, asparagine, aspartic acid, cysteine, glutamine, glutamic acid, glycine, histidine, isoleucine, leucine, lysine, methionine, phenylalanine, proline, threonine, tryptophan, tyrosine or valine. In a particular example, the amino acid substitution is a substitution with alanine, asparagine, aspartic acid, cysteine, glutamine, glutamic acid, glycine, histidine, isoleucine, leucine, lysine, methionine, phenylalanine, proline, tryptophan or valine. Further exemplary polypeptides include those comprising a modification of the tyrosine at the amino acid position corresponding to position 322 of SEQ ID NO:1 and a modification of the serine at the amino acid position corresponding to position 329 of SEQ ID NO:1.

The DNAM-1 polypeptides for expression in T cells also include those lacking all or a portion of the cytoplasmic (or intracellular) domain, for example corresponding to amino acid residues 277 to 336 of SEQ ID NO:1. This domain contains the tyrosine and serine resides at positions corresponding to 322 and 329 of SEQ ID NO:1. DNAM-1 polypeptides lacking all or a portion of this domain, and in particular a portion comprising residues corresponding to residues 322 and 329 of SEQ ID NO:1, may therefore exhibit reduced (including abolished) signalling and facilitate enhanced T cell function.

Precursor human DNAM-1 lacking the cytoplasmic domain (N-terminal signal peptide is in bold):

(SEQ ID NO: 7)
MDYPTLLLALLHVYRALCEEVLWHTSVPFAENMSLEC
VYPSMGILTQVEWFKIGTQQDSIAIFSPTHGMVIRK
PYAERVYFLNSTMASNNMTLFFRNASEDDVGYYSCSL
YTYPQGTWQKVIQVVQSDSFEAAVPSNSHIVSEPGKN
VTLTCQPQMTWPVQAVRWEKIQPRQIDLLTYCNLVHG
RNFTSKFPRQIVSNCSHGRWSVIVIPDVTVSDSGLYR
CYLQASAGENETFVMRLTVAEGKTDNQYTLFVAGGTV
LLLLFVISITTIIVIFLN

Mature human DNAM-1 lacking the cytoplasmic domain:

(SEQ ID NO: 8)
EEVLWHTSVPFAENMSLECVYPSMGILTQVEWFKIGT
QQDSIAIFSPTHGMVIRKPYAERVYFLNSTMASNNMTL
FFRNASEDDVGYYSCSLYTYPQGTWQKVIQVVQSDSFE
AAVPSNSHIVSEPGKNVTLTCQPQMTWPVQAVRWEKIQ
PRQIDLLTYCNLVHGRNFTSKFPRQIVSNCSHGRWSVI
VIPDVTVSDSGLYRCYLQASAGENETFVMRLTVAEGKT
DNQYTLFVAGGTVLLLLFVISITTIIVIFLN

Thus, in some embodiments, the DNAM-1 polypeptides comprise all or a portion of the extracellular domain, for example corresponding to amino acid residues 19 to 248, but optionally lack all or a portion of the cytoplasmic domain. The extracellular domain may comprise all or a portion of the IgG1 domain corresponding to, for example, approximately amino acid residues 19-126 of SEQ ID NO:1, and/or all or a portion of the IgG2 domain corresponding to, for example, approximately amino acid residues 135-239 of SEQ ID NO:1.

Human DNAM-1 extracellular domain:

(SEQ ID NO: 9)
EEVLWHTSVPFAENMSLECVYPSMGILTQVEWFKIGTQ
QDSIAIFSPTHGMVIRKPYAERVYFLNSTMASNNMTL
FFRNASEDDVGYYSCSLYTYPQGTWQKVIQVVQSDSFE
AAVPSNSHIVSEPGKNVTLTCQPQMTWPVQAVRWEKIQ
PRQIDLLTYCNLVHGRNFTSKFPRQIVSNCSHGRWSVI
VIPDVTVSDSGLYRCYLQASAGENETFVMRLTVAEGKT
DNQ

Other exemplary DNAM-1 polypeptides can include those lacking all or a portion of the IgG1 domain corresponding to, for example, approximately amino acid residues 19-126 of SEQ ID NO:1, and/or all or a portion of the IgG2 domain corresponding to, for example, approximately amino acid residues 135-239 of SEQ ID NO:1.

DNAM-1 polypeptides that lack all or a portion of the transmembrane domain corresponding, for example, to amino acid position 249 to 276 of SEQ ID NO:1 are also contemplated. It would be appreciated, however, that to ensure the DNAM-1 polypeptide is expressed on the surface of the T cell and is not secreted, the DNAM-1 polypeptide lacking the endogenous DNAM-1 transmembrane domain comprises an exogenous transmembrane domain. Transmembrane domains from a variety of membrane-bound or transmembrane proteins are known in the art and can be linked to all or a portion of a DNAM-1 extracellular domain. Exemplary transmembrane domains include, but are not limited to, those derived from the transmembrane region(s) of the alpha, beta or zeta chain of the T-cell receptor, CD28, CD3 epsilon, CD45, CD4, CD5, CDS, CD9, CD16, CD22, CD33, CD37, CD64, CD80, CD86, CD 134, CD137 and CD154.

The DNAM-1 polypeptides may be at least or about 80, 90, 100, 110, 120, 130, 140, 150, 160, 170, 180, 190, 200, 210, 220, 230, 240, 250, 260, 270, 280, 290, 300, 310, 230 or 330 amino acids in length. In some examples, the DNAM-1 polypeptides have at least or about 85%, 86%, 87%, 88%, 89%, 90%, 91%, 92%, 93%, 94%, 95%, 96%, 98% or 99% sequence identity with the polypeptides set forth in any one of SEQ ID NOs:5-9 or 21-30, provided they do not have the same sequence as a wild-type DNAM-1 polypeptide (i.e. have less than a 100% sequence identity to a wild-type DNAM-1 polypeptide). The modified DNAM-1 polypeptides of the present disclosure have modifications (e.g. amino acid substitutions, deletions and/or insertions) relative to a wild-type DNAM-1 polypeptide, such as a wild-type human DNAM-1 polypeptide, e.g. one set forth in SEQ ID NO:1 or 2. Consequently, reference herein to any modification is relative to a wild-type DNAM-1 polypeptide. For example, where a modified DNAM-1 polypeptide is said to have an amino acid substitution at a particular position, it is understood that the modified DNAM-1 polypeptide does not comprise the endogenous amino acid residue that is present at that position in a wild-type DNAM-1 polypeptide, i.e. the modified DNAM-1 polypeptide comprises any amino acid residue at that position except for the amino acid residue that is present at that position in the wild-type DNAM-1 polypeptide. For example, a modified DNAM-1 polypeptide that comprises an amino acid substitution of a tyrosine at a position corresponding to position 322 of SEQ ID NO:1 is a modified DNAM-1 polypeptide that comprises alanine, asparagine, aspartic acid, cysteine, glutamine, glutamic acid, glycine, histidine, isoleucine, leucine, lysine, methionine, phenylalanine, proline, serine, threonine, tryptophan or valine at the position corresponding to position 322 of SEQ ID NO:1.

As would be appreciated, the DNAM-1 polypeptides of the present disclosure retain the ability of the wild-type DNAM-1 polypeptide to promote or facilitate T cell function, and in particular anti-tumor activity of the T cell in which it is expressed, i.e. T cells expressing the modified DNAM-1 polypeptide typically have at least the same, and more typically increased, immune function as a T cell expressing a wild-type DNAM-1 polypeptide (e.g. a wild-type human DNAM-1 polypeptide). Methods for assessing the immune function of a T cell expressing a DNAM-1 polypeptide are known in the art and described below.

DNAM-1 polynucleotides encoding a DNAM-1 polypeptide described above and elsewhere herein are also provided, such as DNAM-1 polynucleotides encoding a DNAM-1 polypeptide comprising an amino acid sequence set forth in any one of SEQ ID NOs:1-9 or a polypeptide having at least or about 90%, 91%, 92%, 93%, 94%, 95%, 96%, 97%, 98% or 99% sequence identity thereto. An exemplary polynucleotide encoding the precursor human DNAM-1 polypeptide of SEQ ID NO:1 is set forth in SEQ ID NO:10.

Polynucleotide encoding the precursor human DNAM-1 polypeptide of SEQ ID NO:1 (nucleotides in bold [nucleotides 1-54] encode the signal sequence):

(SEQ ID NO: 10)
atggattatcctactttacttttggctcttcttcatgt
atacagagctctatgtgaagaggtgctttggcatacatc
agttccctttgccgagaacatgtctctagaatgtgtgta
tccatcaatgggcatcttaacacaggtggagtggttcaa
gatcgggacccagcaggattccatagccattttcagccc
tactcatggcatggtcataaggaagccctatgctgagag
ggtttactttttgaattcaacgatggcttccaataacat
gactcttttctttcggaatgcctctgaagatgatgttgg
ctactattcctgctctctttacacttacccacagggaac
ttggcagaaggtgatacaggtggttcagtcagatagttt
tgaggcagctgtgccatcaaatagccacattgtttcgga
acctggaaagaatgtcacactcacttgtcagcctcagat
gacgtggcctgtgcaggcagtgaggtgggaaaagatcca
gccccgtcagatcgacctcttaacttactgcaacttggt
ccatggcagaaatttcacctccaagttcccaagacaaat
agtgagcaactgcagccacggaaggtggagcgtcatcgt
catccccgatgtcacagtctcagactcggggctttaccg
ctgctacttgcaggccagcgcaggagaaaacgaaacctt
cgtgatgagattgactgtagccgagggtaaaaccgataa
ccaatataccctctttgtggctggagggacagttttatt
gttgttgtttgttatctcaattaccaccatcattgtcat
tttccttaacagaaggagaaggagagagagaagagatct
atttacagagtcctgggatacacagaaggcacccaataa
ctatagaagtcccatctctaccagtcaacctaccaatcaat
ccatggatgatacaagagaggatatttatgtcaactatcc
aaccttctctcgcagaccaaagactagagtt

2.2T Cells Expressing Cell-Surface DNAM-1

Provided herein are T cells, including isolated T cells, expressing DNAM-1 on the surface of the cell. Such cells are particularly useful for enhancing immune function (including T cell function) in a subject, treating cancer in a subject, and treating infection in a subject. The T cells expressing DNAM-1 may be CD4+ or CD8+, and/or may be γδ T cells or αβ T cells. In particular embodiments, the T cells are CD8+ T cell.

The T cells can express recombinant DNAM-1, including wild-type DNAM-1 or a variant thereof, such as a modified DNAM-1 described herein. In other embodiments, the T cells do not express recombinant DNAM-1 but simply express endogenous DNAM-1 on the surface. The present disclosure therefore provides a method for preparing a T cell population for adoptive T cell therapy (ACT), comprising introducing into T cell a polynucleotide encoding DNAM-1 so as to produce a population of T cells expressing recombinant DNAM-1, or comprising obtaining a sample of T cells from a subject and selecting DNAM-1 positive (DNAM-1+) T cells (i.e. T cells expressing DNAM-1 on the surface of the cell) from the sample.

2.2.1 T Cells Expressing Recombinant DNAM-1

Provided herein are T cells expressing recombinant and/or modified DNAM-1. Such T cells may therefore have increased levels of surface DNAM-1 compared to T cells that only express endogenous DNAM-1. Accordingly, T cells expressing recombinant DNAM-1 can exhibit enhanced T cell function compared T cells that do not express recombinant DNAM-1 (i.e. T cells that express only endogenous DNAM-1). Thus, the present disclosure also provides methods for enhancing the function of a T cell by introducing a DNAM-1 polynucleotide into the cell so as to express recombinant DNAM-1 in the T cell. Typically, the recombinant DNAM-1 is expressed on the surface of the T cell.

The recombinant DNAM-1 polypeptide expressed in the T cell may be a wild-type DNAM-1 polypeptide or a variant thereof as described above, i.e. a modified DNAM-1 polypeptide described above. Any DNAM-1 polypeptide described herein may be expressed recombinantly in a T cell.

T cells expressing recombinant (including modified) DNAM-1 can be produced using methods well known in the art for generating genetically engineered T cells. In general, DNAM-1 polynucleotides are introduced into a T cell using any one of numerous gene transfer methods. These include, but are not limited to, viral vector gene transfer technologies and non-viral transfer techniques, such as those utilising transposons, mRNA, liposomes, or electroporation or transfection of naked DNA. Exemplary viral vectors for the introduction of a DNAM-1 polynucleotide into a T cell include, without limitation, retrovirus (including lentivirus, gamma retrovirus and alpha retrovirus), adenovirus, adeno-associated virus (AAV), herpes virus (e.g. Cytomegalovirus (CMV)), alphavirus, astrovirus, coronavirus, orthomyxovirus, papovavirus, paramyxovirus (e.g. Sendai virus), parvovirus, picornavirus, poxvirus (e.g. vaccinia virus), and togavirus vectors.

Retroviral vectors are well known in the art and include, for example, vectors derived from B, C and D type retroviruses, xenotropic retroviruses (for example, NZB-X1, NZB-X2 and NZB9-1), polytropic retroviruses e.g., MCF and MCF-MLV, spumaviruses and lentiviruses, for subsequent introduction into a T cell. Exemplary retroviruses for the construction of retroviral vectors include Avian Leukosis Virus, Bovine Leukemia Virus, Murine Leukemia Virus, Mink-Cell Focus-Inducing Virus, Murine Sarcoma Virus, Reticuloendotheliosis Virus and Rous Sarcoma Virus. In some examples, portions of the retroviral vector are derived from different retroviruses. For example, retroviral LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

Recombinant retroviral vectors may be used to generate transduction competent retroviral vector particles by introducing them into appropriate packaging cell lines. Preferably, the recombinant viral vector is a replication defective recombinant virus. Packaging cell lines suitable for use with the above-described retrovirus vectors are well known in the art, are readily prepared (see e.g. WO1995/30763 and WO1992/05266), and can be used to create producer cell lines (also termed vector cell lines or “VCLs”) for the production of recombinant vector particles. Preferably, the packaging cell lines are made from human parent cells (e.g., HT1080 cells) or mink parent cell lines, which eliminates inactivation in human serum. A number of illustrative retroviral systems have been described and can be utilised herein (e.g., U.S. Pat. Nos. 5,219,740; 6,207,453; 5,219,740; Miller and Rosman, 1989, BioTechniques 7:980-990; Miller, A. D., 1990, Human Gene Therapy 1:5-14; Scarpa et al. 1991, Virology 180:849-852; Burns et al. 1993, Proc. Natl. Acad. Sci. USA 90:8033-8037; and Boris-Lawrie and Temin, 1993, Cur. Opin. Genet. Develop. 3: 102-109). In a particular example, a lentiviral vector is used. Exemplary methods and vectors for lentiviral-based gene transfer are known in the art and are described in, e.g., Wang et al. 2012, J. Immunother. 35(9): 689-701; Cooper et al. 2003, Blood. 101: 1637-1644; Verhoeyen et al. (2009) Methods Mol Biol. 506: 97-114; and Cavalieri et al. 2003, Blood. 102(2): 497-505.

In other embodiments, recombinant polynucleotides are transferred into T cells via electroporation (see, e.g., Chicaybam et al, 2013, PLoS ONE 8(3): e60298 and Van Tedeloo et al. 2000, Gene Therapy 7(16): 1431-1437), optionally with CRISPR-Cas9 to target the insertion (Roth et al., 2018, Nature, 559:405-409). In some embodiments, recombinant nucleic acids are transferred into T cells via transposition (see, e.g., Manuri et al. 2010, Hum Gene Ther 21(4): 427-437; Sharma et al. 2013, Molec Ther Nucl Acids 2, e74; and Huang et al. 2009, Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection, tungsten particle-facilitated microparticle bombardment (Johnston, 1990, Nature, 346: 776-777), and strontium phosphate DNA co-precipitation (Brash et al., 1987, Mol. Cell Biol., 7: 2031-2034).

As would be understood, the DNAM-1 polynucleotide is generally operably linked to a promoter for subsequent introduction and expression in T cells. Additional promoter elements, e.g., enhancers, regulate the frequency of transcriptional initiation and can also be utilised. Promoters and enhancers for use in transgene expression in mammalian cells are well known in the art and any such promoter can be used to express DNAM-1 in a T cell. Exemplary promoters for expression of polynucleotides in T cells include the CMV IE gene, EF1a, ubiquitin C, or phosphoglycerokinase (PGK) promoters. The EF1a promoter has been extensively used in mammalian expression plasmids and has been shown to be effective in driving expression from nucleic acid molecules cloned into vector. Another example of a promoter is the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-Ia promoter, the hemoglobin promoter, and the creatine kinase promoter.

Vectors may also include, for example, a polyadenylation signal and transcription terminator (e.g., from Bovine Growth Hormone (BGH) gene), an element allowing episomal replication and replication in prokaryotes (e.g. SV40 origin and ColEl or others known in the art) and/or elements to allow selection (e.g., ampicillin resistance gene and/or zeocin marker).

2.2.2 Sourcing and Preparation of T Cells

To produce the T cells of the present disclosure, cells are typically obtained or derived from a biological sample from a subject (e.g. a human subject or non-human animal subject, e.g. mouse, rat, rabbit, pig, chimpanzee etc.) using methods well known in the art. In some examples, the cells are isolated and/or otherwise prepared from the subject who is to receive the cell therapy, or from a sample derived from such a subject, i.e. the cells are autologous. In other examples, the cells are isolated and/or otherwise prepared from a subject other than a subject who is to receive or who ultimately receives the cell therapy, i.e. the cells are allogeneic or xenogeneic. Typically, the cells are primary T cells, although T cells from T cell lines generated from a biological sample are also contemplated.

The sample from which the cells are obtained includes, for example, tissue, fluid, and other samples taken directly from the subject, as well as samples resulting from one or more processing steps, such as separation, centrifugation, genetic engineering (e.g. transduction with viral vector), washing, and/or incubation. Exemplary samples include whole blood, peripheral blood mononuclear cells, bone marrow, lymph node tissue, cord blood, thymus tissue, tissue from a site of infection, ascites, pleural effusion, spleen tissue, and tumor tissue, and/or cells derived therefrom. In some aspects, the sample from which the cells are derived or isolated is blood or a blood-derived sample, or is or is derived from an apheresis or leukapheresis product.

Isolation of T cells can include one or more preparation and/or non-affinity based cell separation steps. In some examples, cells are washed, centrifuged, and/or incubated in the presence of one or more reagents, for example, to remove unwanted components, enrich for desired components, or lyse or remove cells sensitive to particular reagents. Cells can be separated based on one or more property, such as density, adherent properties, size, sensitivity and/or resistance to particular components.

In some examples, cells from the circulating blood of a subject are obtained, e.g., by apheresis or leukapheresis. Typically, blood cells collected from the subject are washed to remove the plasma fraction and placed in an appropriate buffer or media for subsequent processing steps. In some embodiments, the cells are washed with phosphate buffered saline (PBS). In some embodiments, the wash solution lacks calcium and/or magnesium and/or many or all divalent cations. Washing step steps can be accomplished using a semi-automated “flow-through” centrifuge (for example, the Cobe 2991 cell processor, the Baxter CytoMate, or the Haemonetics Cell Saver 5) according to the manufacturer's instructions. In some aspects, a washing step is accomplished by tangential flow filtration (TFF) according to the manufacturer's instructions. After washing, the cells may be resuspended in a variety of biocompatible buffers. Alternatively, the undesirable components of the apheresis sample may be removed and the cells directly resuspended in culture media.

Methods of isolating T cells can also include density-based cell separation methods, such as the preparation of white blood cells from peripheral blood by lysing the red blood cells and centrifugation through a Percoll or Ficoll gradient. In further embodiments, one or more steps that separate different cell types based on the expression of one or more markers, such as surface proteins, intracellular markers, or nucleic acid, are included in the methods. Any known method for separation based on such markers may be used, including, for example, affinity- or immunoaffinity-based separation. For example, separation of cells and cell populations based on the expression or expression level of one or more markers, such as cell surface markers, can be achieved by incubation with an antigen-binding molecule that specifically binds to such markers, followed generally by washing steps and separation of cells having bound the antigen-binding molecule from those cells having not bound to the antigen-binding molecule.

Such separation steps can be based on positive selection, in which the cells having bound the antigen-binding molecule are retained for further use, and/or negative selection, in which the cells having not bound to the antigen-binding molecule are retained. As would be appreciated, separation need not result in 100% enrichment or removal of a particular cell population or cells expressing a particular marker. In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antigen-binding molecules, each specific for a marker targeted for negative selection. Likewise, multiple cell types can simultaneously be positively selected by incubating cells with a plurality of antigen-binding molecule expressed on the various cell types.

For the purposes of the present disclosure, T cells can be selected based on expression of CD3. In some embodiments, T cells are separated from a PBMC sample by negative selection of markers expressed on non-T cells, such as B cells, monocytes, or other white blood cells, such as CD14. In some instances, no further selection is made, such that the T cell population comprises all T cells in the sample, including, for example, CD4+ and CD8+ T cells, DNAM-1− and DNAM-1+ T cells, and all other phenotypes of T cells. In other examples, selection is used to isolate a more particular subpopulation of T cells.

In one embodiment of the present disclosure, DNAM-1+ T cells are selected. Thus, the present disclosure provides methods for preparing a T cell population for adoptive cell therapy by obtaining a sample of T cells from a subject and selecting DNAM-1+ T cells from the sample. The level of surface expression of DNAM-1 can be assessed and taken into account when selecting DNAM-1+ T cells. For example, DNAM-1+ T cells can be separated into those expressing “low” and “high”, or “low”, “medium” and “high” levels of DNAM-1 with respect to other DNAM-1+ T cells in the population, such as by using flow cytometry as is well known in art. One or more of the isolated subpopulations of DNAM-1+ T cells can then be retained for use in the methods described herein.

In further embodiments, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. Thus, T cells of the present disclosure include CD4+ T cells and CD8+ T cells. In particular embodiments, T cells of the present disclosure are CD8+ T cells.

CD8+ T cells can be further enriched for or depleted of naive, central memory, effector memory, and/or central memory stem cells, such as by positive or negative selection based on surface antigens associated with the respective subpopulation. For example, enrichment for central memory T (TCM) cells may be carried out to increase efficacy, such as to improve long-term survival, expansion, and/or engraftment following administration, which in some aspects is particularly robust in such sub-populations (see e.g. Terakura et al. 2012, Blood.1:72-82; Wang et al. 2012, J Immunother. 35(9):689-701. In some embodiments, the enrichment for TCM cells is based on positive or high surface expression of CD45RO, CD62L, CCR7, CD28, CD3, and/or CD 127; in some embodiments, it is based on negative selection for cells expressing or highly expressing CD45RA and/or granzyme B. Thus, isolation of a CD8+ population enriched for TCM cells can be carried out by depletion of cells expressing CD4, CD14, CD45RA, and positive selection or enrichment for cells expressing CD62L.

Isolated T cells can be incubated and/or cultured using any method known in the art. The incubation steps can include culture, cultivation, stimulation, activation, and/or propagation. In some embodiments, the cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a DNAM-1 polynucleotide and/or other polynucleotide, such as one encoding a chimeric antigen receptor as expanded on below.

In some embodiments, the stimulating conditions (e.g. to facilitate expansion of the cells) include exposure to one or more agent, e.g., ligand, that is capable of activating an intracellular signaling domain of a TCR complex. For example, the agent may initiate the TCR/CD3 intracellular signaling cascade in a T cell. Such agents can include antibodies, such as those specific for a TCR component and/or costimulatory receptor (e.g., anti-CD3, anti-CD28 antibodies) bound to solid support such as a bead, and/or one or more cytokines. Optionally, the expansion method may further include the step of adding anti-CD3 and/or anti CD28 antibody to the culture medium. In some embodiments, the stimulating agents include IL-2 and/or IL-15. Methods for the culture and expansion of T cells are known in the art and include, for example, those described in in U.S. Pat. No. 6,040,177, Klebanoff et al. 2012, J Immunother. 35(9): 651-660, Terakuraet al. 2012 Blood. 1:72-82, and Wang et al. 2012, J Immunother. 35(9):689-701.

In some embodiments, the T cells are expanded by adding them to culture-initiating composition feeder cells, such as non-dividing peripheral blood mononuclear cells (PBMC), and incubating the culture for a time sufficient to expand the numbers of T cells. The non-dividing feeder cells may comprise gamma-irradiated PBMC feeder cells. In further embodiments, the incubation may further include adding non-dividing EBV-transformed lymphoblastoid cells (LCL) as feeder cells, which may optionally be irradiated with gamma rays. In embodiments, antigen-specific T cells are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

The cells may incubated and/or cultured prior to or in connection with genetic engineering, such as engineering to express recombinant DNAM-1 as described above. Moreover, the T cells expressing DNAM-1, including recombinant and/or endogenous DNAM-1, can also express one or more other recombinant polypeptides. In embodiments where the T cells express recombinant DNAM-1, the other recombinant polypeptide(s) can be engineered into the T cell before, simultaneously or after the T cell is modified to express the recombinant DNAM-1. In particular examples, exemplary T cells of the present disclosure express a recombinant receptor, including a transgenic T cell receptor (TCR) or a chimeric antigen receptor (CAR).

In a particular embodiment, the T cells described herein express recombinant antigen receptors, such as CARs, i.e. are CAR T cells. Exemplary antigen receptors, including CARs, and methods for engineering and introducing such receptors into cells, include those described, for example, in international patent application publication numbers WO200014257, WO2013126726, WO2012129514, WO2014031687, WO2013166321, WO2013071154, WO2013123061 U.S. patent application publication numbers US2002131960, US2013287748, US20130149337, U.S. Pat. Nos.: 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and/or those described by Sadelain et al., 2013, Cancer Discov. 3(4): 388-398; Davila et al., 2013, PLoS ONE 8(4): e61338; Turtle et al., 2012, Curr. Opin. Immunol., 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO2014055668.

CARs include an extracellular antigen-binding domain, a transmembrane domain, and an intracellular signaling domain (or cytoplasmic domain). The extracellular antigen binding domain may be a receptor or domain of a receptor that binds to a ligand or may be an antibody or antigen-binding portion thereof, such a variable heavy (VH) chain region and/or variable light (VL) chain region of the antibody, e.g., a scFv antibody fragment. In some embodiments, the antigen-binding domain further includes at least a portion of an immunoglobulin constant region, such as a hinge region, e.g., an IgG4 hinge region, and/or a CH1/CL and/or Fc region. The constant region or portion is generally of a human IgG, such as IgG4 or IgG1. The portion of the constant region may serve as a spacer region between the antigen-recognition component, e.g., scFv, and transmembrane domain of the CAR. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. 2013, Clin. Cancer Res., 19:3153, International patent application publication number WO2014031687 and U.S. Pat. No. 8,822,647.

The antigen-binding domain generally binds to a tumor antigen. Exemplary tumor antigens to which the antigen-binding region binds include, but are not limited to, TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-1Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-Ia, MAGE-A1, legumain, HPV E6, E7, MAGEA1, ETV6-AML, sperm protein 17, XAGE1, Tie 2, MAD-CT-1, MAD-CT-2, Fos-related antigen 1, p53, p53 mutant, prostein, survivin and telomerase, PCTA-I/Galectin 8, MelanA/MART-1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In some embodiments, the antigen-binding domains comprises an antigen-binding domain from NKG2D, NKG2A, NKG2C, NKG2F, LLT1, AICL, CD26, NKRP1, NKp30, NKp44, NKp46, CD244 (2B4), DNAM-1, and NKp80. In particular embodiments, however, the antigen-binding domain of the CAR does not comprise an antigen-binding domain of DNAM-1 (e.g. the extracellular domain of DNAM-1). Thus, in some embodiments, the T cells of the present disclosure do not contain a DNAM-1 polypeptide linked to, or comprising, an exogenous intracellular signaling domain that can mimic activation through an antigen receptor complex as described below (i.e. in some embodiments, the DNAM-1 polypeptide expressed on the T cell is not linked to or does not comprise an exogenous intracellular signaling domain that can mimic activation through an antigen receptor complex).

The intracellular signaling domain comprises one or more intracellular signaling components, such as signaling components that mimic activation through an antigen receptor complex, such as a TCR complex, and/or signal via another cell surface receptor. The signal may be immunostimulatory and/or costimulatory in some embodiments. Thus, an intracellular signaling domain is generally responsible for activation of at least one of the normal effector functions of the immune cell in which the CAR has been introduced (e.g., in the case of T cells, cytolytic activity or helper activity including the secretion of cytokines).

Examples of intracellular signaling domains for use in CARs are well known in the art and include the cytoplasmic sequences of the T cell receptor (TCR) and co-receptors that act in concert to initiate signal transduction following antigen receptor engagement, as well as any derivative or variant of these sequences and any recombinant sequence that has the same functional capability. As signals generated through the TCR alone are insufficient for full activation of the T cell, a secondary and/or costimulatory signal is may also be included. Thus, CAR can include a primary intracellular signaling domain that initiates antigen-dependent primary activation through the TCR and a secondary cytoplasmic domain or costimulatory domain that acts in an antigen-independent manner to provide a secondary or costimulatory signal.

Primary intracellular signaling domains that act in a stimulatory manner may contain signaling motifs which are known as immunoreceptor tyrosine-based activation motifs or ITAMs. Examples of ITAM containing-primary intracellular signaling domains that have been used to generate CARS include those of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. Exemplary costimulatory signaling domains are those that comprise the intracellular domain of a costimulatory molecule, i.e. a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1, SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a. As would be appreciated, CARs may comprise 2 or more costimulatory signaling domains, such as 2, 3, 4, 5, 6, 7, 8 or more.

The transmembrane domain of the CAR may be derived either from a natural or a synthetic source. Where the source is natural, the domain in some aspects is derived from any membrane-bound or transmembrane protein. Transmembrane regions include those derived from the transmembrane region(s) of the alpha, beta or zeta chain of the T-cell receptor, CD28, CD3 epsilon, CD45, CD4, CD5, CDS, CD9, CD16, CD22, CD33, CD37, CD64, CD80, CD86, CD134, CD137 and CD154.

In some examples, the T cells are TCR-deficient. TCR-deficient T cells include those lacking a functional TCR (e.g., T cells engineered such that they do not express any functional TCRs on the cell surface, engineered such that they do not express one or more subunits that comprise a functional TCR, or engineered such they produce very little functional TCR on the cell surface) and those expressing substantially impaired TCRs (e.g., by expression of mutated or truncated forms of one or more of the subunits of the TCR). TCR-deficient T cells include those described in U.S. Pat. No. 9,663,763 and United States Patent Publication No. 20070036773. Such T cells can be produced, for example, by targeting nucleic acids encoding specific TCRs, such as TCR-α and TCR-β, and/or CD3 chains (e.g., CD3 zeta), such as by the introduction of small-hairpin RNAs (shRNAs) into the T cell that target the nucleic acids, or the use of zinc finger nucleases, transcription activator-like effector nucleases (TALENs) or the CRISPR/Cas9 system to disrupt endogenous TCRs.

In other embodiments, TCR-deficient cells, such as those described in U.S. Pat. No. 9,663,763, are explicitly excluded from the present invention, i.e. in some embodiments, the T cells of the present disclosure express functional TCRs. The functional TCRs may be endogenous or recombinant TCRs.

The biological activity of the T cells may be measured by any of a number of known methods. The activity of the T cells can be assessed in vitro, in vivo (e.g. using an animal model of disease, such as an animal model of cancer or infection), or ex vivo. Parameters to assess include specific binding of a T cell to an antigen by ELISA or flow cytometry. In certain embodiments, the ability of the T cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In particular embodiments, the biological activity of the cells is measured by assaying expression and/or secretion of certain proteins (i.e. T cell function biomarkers), such as CD107a, IFN-γ, IL-2, TNF and Ki67. For example, IFN-γ, IL-2, and TNF can be used as biomarkers for CD8+ T cell activation; CD107a can be used a marker for degranulation; and Ki67 can be used as a biomarker for T cell proliferation.

Any method known in the art to detect T cell function biomarkers can be used in accordance with the present disclosure. Such methods include, but are not limited to, of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof.

The biological activity can also, or alternatively, be measured by assessing clinical outcome, such as reduction in tumor burden or load. For example, small animal models of cancer (e.g. mice harbouring a tumor) can be injected with T cells of the present disclosure and the tumor burden can be monitored and assessed (such as described in the Examples below).

### 3. Pharmaceutical Compositions and Formulations

Also provided herein are pharmaceutical compositions and formulations comprising a T cell of the present disclosure and a pharmaceutically acceptable carrier. The pharmaceutical compositions may also comprise one or more other active agents, such as one or more chemotherapeutic agents or one or more anti-infective agents. Non-limiting examples of these are detailed in the section below and any one or more can be included in the pharmaceutical compositions of the present disclosure.

Pharmaceutical compositions and formulations as described herein can be prepared by mixing the active ingredients (e.g., a small molecule, nucleic acid, or polypeptide) having the desired degree of purity with one or more optional pharmaceutically acceptable carriers (Remington's Pharmaceutical Sciences 16th edition, Osol, A. Ed. (1980)). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and metal complexes (e.g., Zn-protein complexes); and/or non-ionic surfactants such as polyethylene glycol (PEG).

In one embodiment, the pharmaceutically acceptable carrier is suitable for parenteral administration. Alternatively, the carrier can be suitable for intravenous, intraperitoneal, intramuscular, or sublingual administration. In particular examples, pharmaceutically acceptable carriers include sterile aqueous solutions or dispersions for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the invention is contemplated. In a particular embodiment, appropriate carriers include, but are not limited to, Hank's Balanced Salt Solution (HBSS) and Phosphate Buffered Saline (PBS).

As would be appreciated, pharmaceutical compositions typically must be sterile and stable under the conditions of manufacture and storage. For the purposes of the present disclosure, the pharmaceutical composition is formulated as a solution. The T cells and optionally one or more other agents can be administered by a variety of dosage forms. Accordingly, the pharmaceutical compositions may be formulated as single or multidose preparations. Any biologically-acceptable dosage form known to persons of ordinary skill in the art, and combinations thereof, are contemplated. Examples of such dosage forms include, without limitation, liquids, solutions, suspensions, emulsions, injectables (including subcutaneous, intramuscular, intravenous, and intradermal), infusions, and combinations thereof.

### 4. Therapeutic Uses

The present disclosure provides methods for enhancing immune function (including T cell function) in a subject, methods for treating cancer in a subject, and methods for treating and infection, by administering to a subject a T cell described herein that expresses DNAM-1. Thus, the T cells of the present disclosure can be administered to a subject as part of an adoptive cell transfer therapy for enhancing immune function in the subject, such as to treat cancer or an infection.

Methods for administration of cells for adoptive cell therapy are known and may be used in connection with the present disclosure. For example, adoptive T cell therapy methods are described, e.g., in US Patent Application Publication No. 2003/0170238; U.S. Pat. No. 4,690,915; Rosenberg, 2011, Nat Rev Clin Oncol. 8(10):577-85); Themeli et al. 2013, Nat Biotechnol. 31(10): 928-933; Tsukahara et al. 2013, Biochem Biophys Res Commun 438(1): 84-9; Davila et al. 2013, PLoS ONE 8(4): e61338.

Adoptive cell therapy can be carried out by autologous transfer, in which the cells are isolated and/or otherwise prepared from the subject who is to receive the cell therapy, or from a sample derived from such a subject. Thus, in some aspects, the T cells of the present disclosure are derived from a subject in need of a treatment and the cells, following isolation and processing, are administered to the same subject. In other embodiments, the cell therapy is carried out by allogeneic transfer, in which the cells are isolated and/or otherwise prepared from a subject other than a subject who is to receive or who ultimately receives the cell therapy. In such embodiments, the cells are derived from a first subject then are administered to a different second subject of the same species. In some embodiments, the first and second subjects are genetically identical or similar. For example, the second subject may express the same HLA class or supertype as the first subject. Xenogeneic transfer is also contemplated, wherein the T cells are cells are isolated and/or otherwise prepared from a subject of a different species to the subject who is to receive the cell therapy.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration. In some embodiments, a given dose is administered by a single bolus administration of the cells. In other embodiments, multiple bolus administration of the cells is performed, for example, over a period of no more than 3 days, or by continuous infusion administration of the cells.

The appropriate dosage may be determined based on the type of disease to be treated, the type of T cell, the severity and course of the disease, the clinical condition of the subject, the subject's clinical history and response to the treatment, and the discretion of the attending physician. Dosages can be empirically determined considering the type and stage of disease diagnosed in a particular patient. The dose administered to a patient, in the context of the present disclosure, should be sufficient to effect a beneficial therapeutic response in the subject over time. The size of the dose also will be determined by the existence, nature, and extent of any adverse side-effects that accompany the administration of a particular compound in a particular patient. Determination of the proper dosage for a particular situation is within the skill of the practitioner. In some embodiments, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch or pump.

In one embodiment, the T cells are administered to a subject at an amount of between about 10⁵ to 10¹¹ cells, such as at least or about 10⁵, 10⁶, 10⁷, 10⁸, 10⁹, 10¹⁰, or 10¹¹ cells. In a particular embodiment, the T cells are administered at an amount of between 10⁸ to 10⁹ cells. The T cells may be administered at any frequency deemed therapeutic and safe, such as at a frequency of one or more times a week (e.g. daily, or 2, 3, 4, 5 or 6 times a week), or once every 2, 3, 4, 5, 6, 7, 8, 9, 10, 111, 12, 13, 14, 15, 16, 17, 18, 19, 20 or more weeks.

The T cells of the present disclosure can be administered alone or in conjunction with one or more other therapies, including one or more anti-cancer therapies (e.g. surgery, radiation therapy or chemotherapy) for the treatment of cancer, or one or more anti-infective therapies for the treatment of infection. Exemplary therapies include radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. In some embodiments, the additional therapy is radiation therapy. In other embodiments, the additional therapy is surgery. In particular embodiments, the additional therapy is a combination of radiation therapy and surgery. In some embodiments, the additional therapy is gamma irradiation. The subject can be exposed to the one or more other therapies before and/or after the T cells of the present disclosure. In some embodiments, the subject is exposed to the one or more other therapies at the same time as being administered the T cells. In such embodiments, the T cells and the additional therapy may be, for example, in the same formulation (such as a pharmaceutical composition described above) or in different formulations.

Non-limiting examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, canninonnycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylannine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agents also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Anti-cancer antibodies are also chemotherapeutics and can be utilised in the methods and compositions herein. Such antibodies include, but are not limited to, alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG.sub.1.lamda. antibody genetically modified to recognize interleukin-12 p40 protein.

Chemotherapeutic agents also includes EGFR inhibitors, which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAID 455 (ATCC CRL HB8507), MAb 225 (ATCC CRL 8508), MAb 528 (ATCC CRL 8509) (see, U.S. Pat. No. 4,943,533) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-α for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., 2004, J. Biol. Chem. 279(29):30375-30384). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)annino]-7-[3-(4-morpholinyl)propoxy]-6-quin-azolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoli-ne, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrinnido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Other chemotherapeutic agents are tyrosine kinase inhibitors, including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor α (TNF-α) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T-cell costimulation blockers such as abatacept (Orencia), Interleukin 6 (IL-6) blockers such as tocilizumab (ACTEMERA®); Interleukin 13 (IL-13) blockers such as lebrikizumab; Interferon α (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin α (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In other examples, the T cells of the present disclosure are administered to the subject in conjunction with an anti-infective drug. The anti-infective drugs is suitably selected from antimicrobials, which include without limitation compounds that kill or inhibit the growth of microorganisms such as viruses, bacteria, yeast, fungi, protozoa, etc. and thus include antibiotics, amebicides, antifungals, antiprotozoals, antimalarials, antituberculotics and antivirals. Anti-infective drugs also include within their scope anthelmintics and nematocides. Illustrative antibiotics include quinolones (e.g., amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin; gemifloxacin; and garenoxacin), tetracyclines, glycylcyclines and oxazolidinones (e.g., chlortetracycline, demeclocycline, doxycycline, lymecycline, methacycline, minocycline, oxytetracycline, tetracycline, tigecycline; linezolide, eperozolid), glycopeptides, aminoglycosides (e.g., amikacin, arbekacin, butirosin, dibekacin, fortimicins, gentamicin, kanamycin, meomycin, netilmicin, ribostamycin, sisomicin, spectinomycin, streptomycin, tobramycin), □-lactams (e.g., imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephaacetrile, cephalexin, cephaloglycin, cephaloridine, cephalothin, cephapirin, cephradine, cefinetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g., azithromycin, clarithromycin, erythromycin, oleandomycin, rokitamycin, rosaramicin, roxithromycin, troleandomycin), ketolides (e.g., telithromycin, cethromycin), coumermycins, lincosamides (e.g., clindamycin, lincomycin) and chloramphenicol. Illustrative antivirals include abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivudine/zidovudine, nelfinavir mesylate, nevirapine, oseltamivir phosphate, ribavirin, rimantadine hydrochloride, ritonavir, saquinavir, saquinavir mesylate, stavudine, valacyclovir hydrochloride, zalcitabine, zanannivir, and zidovudine. Non-limiting examples of amebicides or antiprotozoals include atovaquone, chloroquine hydrochloride, chloroquine phosphate, metronidazole, metronidazole hydrochloride, and pentamidine isethionate. Anthelmintics can be at least one selected from mebendazole, pyrantel pamoate, albendazole, ivermectin and thiabendazole. Illustrative antifungals can be selected from amphotericin amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin, and terbinafine hydrochloride. Non-limiting examples of antimalarials include chloroquine hydrochloride, chloroquine phosphate, doxycycline, hydroxychloroquine sulfate, mefloquine hydrochloride, primaquine phosphate, pyrimethamine, and pyrimethamine with sulfadoxine. Antituberculotics include but are not restricted to clofazimine, cycloserine, dapsone, ethambutol hydrochloride, isoniazid, pyrazinamide, rifabutin, rifampin, rifapentine, and streptomycin sulfate.

In some embodiments, the subject to whom the T cells are administered has cancer. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In specific embodiments, the cancer is melanoma or lung cancer, suitably metastatic melanoma or metastatic lung cancer.

In some embodiments, the individual has cancer that is resistant to one or more immunotherapies, including one or more immune checkpoint inhibitors, including a PD-1 inhibitor, PD-L1 inhibitor or a CTLA-4 inhibitor. Resistance to an inhibitor may manifest as recurrence of cancer or refractory cancer. Recurrence may refer to the reappearance of cancer, in the original site or a new site, after treatment. In some embodiments, resistance to an immune checkpoint inhibitor manifests as progression of the cancer during treatment with the inhibitor. In some embodiments, resistance to a immune checkpoint inhibitor results in cancer that does not respond to treatment. The cancer may be resistant at the beginning of treatment or it may become resistant during treatment. In some embodiments, the cancer is at early stage or at late stage.

In a particular embodiments of the present disclosure, subjects may undergo leukapheresis, wherein leukocytes are collected, enriched, or depleted ex vivo to select and/or isolate the cells of interest, e.g., T cells, as essentially described above. These T cell isolates may be expanded by methods known in the art and optionally engineered to express recombinant DNAM-1 and/or other recombinant molecules (e.g. TCRs or CARs). Subjects in need thereof may subsequently undergo standard treatment with high dose chemotherapy followed by peripheral blood stem cell transplantation. In certain aspects, following or concurrent with the transplant, subjects receive an infusion of the expanded T cells of the present disclosure. In an additional aspect, expanded cells are administered before or following surgery.

In some embodiments, the subject has an infection and the T cells of the present disclosure are administered to the subject for treatment of the infection. Infections include, but are not limited to, those caused by viruses, prions, bacteria, viroids, parasites, protozoans and fungi. Non-limiting examples of viruses include Retroviridae human immunodeficiency viruses, such as HIV-1 (also referred to as HTLV-III, LAV or HTLV-III/LAV, or HIV-III); and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis, including Norwalk and related viruses); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus, Metapneumovirus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, VACV, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); and astroviruses. Representative bacteria that are known to be pathogenic include pathogenic Pasteurella species (e.g., Pasteurella multocida), Staphylococcus species (e.g., Staphylococcus aureus), Streptococcus species (e.g., Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae), Neisseria species (e.g., Neisseria gonorrhoeae, Neisseria meningitidis), Escherichia species (e.g., enterotoxigenic E. coli (ETEC), enteropathogenic E. coli (EPEC), enterohemorrhagic E. coli (EHEC), and enteroinvasive E. coli (EIEC)), Bordetella species, Campylobacter species, Legionella species (e.g., Legionella pneumophila), Pseudomonas species, Shigella species, Vibrio species, Yersinia species, Salmonella species, Haemophilus species (e.g., Haemophilus influenzae), Brucella species, Francisella species, Bacteroides species, Clostridiium species (e.g., Clostridium difficile, Clostridium perfringens, Clostridium tetani), Mycobacteria species (e.g., M. tuberculosis, M. avium, M. intracellulare, M. kansaii, M. gordonae), Helicobacter pyloris, Borelia burgdorferi, Listeria monocytogenes, Chlamydia trachomatis, Enterococcus species, Bacillus anthracis, Corynebacterium diphtheriae, Erysipelothrix rhusiopathiae, Enterobacter aerogenes, Klebsiella pneumoniae, Fusobacterium nucleatum, Streptobacillus moniliformis, Treponema pallidium, Treponema pertenue, Leptospira, Rickettsia, and Actinomyces israeli. Non-limiting pathogenic fungi include Cryptococcus neoformans, Histoplasma capsulatum, Coccidioides immitis, Blastomyces dermatitidis, Candida albicans, Candida glabrata, Aspergillus fumigata, Aspergillus flavus, and Sporothrix schenckii. Illustrative pathogenic protozoa, helminths, Plasmodium, such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

Once the cells are administered to the subject, the biological activity of the T cells (e.g. T cell activation, T cell proliferation, cytolytic activity, and/or anti-tumor activity) may be measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In particular embodiments, the biological activity of the cells is measured by assaying expression and/or secretion of certain proteins (e.g. T cell function biomarkers), such as CD107a, IFN-γ, IL-2, TNF and Ki67. The biological activity can also, or alternatively, be measured by assessing clinical outcome, such as reduction in tumor burden or load. In some aspects, toxic outcomes, persistence and/or expansion of the cells, and/or presence or absence of a host immune response, are assessed.

Any method known in the art to detect T cell function biomarkers can be used in accordance with the present disclosure. Such methods include, but are not limited to, FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof.

In some embodiments, any one or more of the T cell function biomarkers are detected in the sample by protein expression. In some embodiments, protein expression is determined by immunohistochemistry (IHC). In some embodiments, any one or more of the T cell function biomarkers are detected using an antibody that binds specifically to the biomarker.

In particular embodiments, activated CD8+ T cells in the subject are assessed by detecting and/or measuring IFN-γ producing CD8+ T cells and/or enhanced cytolytic activity as compared to before the administration of T cells. IFN-γ may be measured by any means known in the art, including, e.g., intracellular cytokine staining (ICS) involving cell fixation, permeabilization, and staining with an antibody against IFN-γ. Cytolytic activity may be measured by any means known in the art, e.g., using a cell killing assay with mixed effector and target cells. In other embodiments, the release of cytokines such as IFN-γ, TNF-α and interleukins such as IL-2 is assessed as a marker of activated CD8+ T-cells. Cytokine release may be measured by any means known in the art, e.g., using Western blot, ELISA, or immunohistochemical assays to detect the presence of released cytokines in a sample containing T-cells. In further embodiments, Tcell proliferation is detected by determining percentage of Ki67+CD8+ T cells (e.g., by FACS analysis). In some embodiments, T cell proliferation is detected by determining percentage of Ki67+CD4+ T cells (e.g., by FACS analysis). In some embodiments, the T cells are from peripheral blood. In other embodiments, the T cells are from a tumor.

### 5. Diagnostic Uses

As demonstrated herein, DNAM-1 is important for immune function of T cells in the tumor environment, and is prognostic of cancer survival and responsiveness to cancer therapy (e.g. immune checkpoint inhibitor therapy). Accordingly, DNAM-1 can be used as a biomarker of T cell function. Thus, the present disclosure also provides methods for assessing the immune function of a subject, and/or the immune function of T cells (e.g. CD4+ or CD8+ T cells) in a subject by determining the amount or level of DNAM-1 on T cells obtained from the subject and/or the number or percentage of DNAM-1+ T cells in a population. The present disclosure also provides methods for predicting the likelihood that a subject will survive cancer, or the survival time of a subject with cancer, by determining the expression level of DNAM-1 in T cells obtained from the subject, the amount or level of DNAM-1 on T cells obtained from the subject and/or the number or percentage of DNAM-1+ T cells in a population. Also provided are methods for predicting the likelihood that a subject with cancer will respond to cancer therapy, such as with an immune checkpoint inhibitor, by determining the expression level of DNAM-1 in T cells obtained from the subject, the amount or level of DNAM-1 on T cells obtained from the subject and/or the number or percentage of DNAM-1+ T cells in a population. In some embodiments, surface DNAM-1 levels are used as a biomarker for immune function of T cells, cancer survival and/or responsiveness to therapy. In particular embodiments, the number of DNAM-1+ T cells in a population are assessed and used as a biomarker (e.g. the number or percentage of T cells that are positive for surface DNAM-1). In a still further embodiment, the number or percentage of DNAM-1+ CD8+ T cells is assessed, e.g. the number of tumor infiltrating DNAM-1+CD8+ T cells per total CD8+ T cells. In further embodiments, expression levels of DNAM-1 are used as a biomarker for cancer survival.

T cells can be obtained from T cell-containing patient samples which are suitably selected from tissue samples such as tumors and fluid samples such as peripheral blood. In some embodiments, the sample is obtained prior to, during and/or after treatment with a therapeutic composition. Thus, in particular embodiments, the methods can be used to monitor the immune function of T cells during and after treatment and thus, in some example, the effectiveness of treatment. In some embodiments, the tissue sample is formalin fixed and paraffin embedded, archival, fresh or frozen.

The level or amount of DNAM-1, or DNAM-1+ cells, can be determined qualitatively and/or quantitatively based on any suitable criterion known in the art, including but not limited to DNA, mRNA, cDNA, proteins, protein fragments and/or gene copy number. In particular embodiments, the DNAM-1 expression levels, the amount of DNAM-1 protein on the surface of T cells or the number of DNAM-1+ T cells is assessed quantitatively. In some examples, the DNAM-1 expression levels, the amount of DNAM-1 on the T cells or the number of DNAM-1+ T cells in a sample from a subject is compared to a second sample that is a reference sample, reference cell, reference tissue, control sample, control cell, or control tissue, and for which the DNAM-1 level is known to correlate with a particular phenotype (e.g. immune function (e.g. effective immune function, or ineffective or impaired immune function), responsiveness to therapy (e.g. complete, partial or non-responsiveness to therapy), or survival time (e.g. in months, or years)). In other examples, the DNAM-1 expression levels, the amount of DNAM-1 on the T cells and/or the number of DNAM-1+ T cells in a sample from a subject is compared to a reference level/amount/number, where the reference level is known to correlate with a particular phenotype or is a cut-off, above or below which is known to correlate with a particular phenotype (e.g. immune function (e.g. effective immune function, or ineffective or impaired immune function), responsiveness to therapy (e.g. complete, partial or non-responsiveness to therapy), or survival time (e.g. in months, or years)). By comparing the level or amount of DNAM-1 or DNAM-1+ cells in a sample to a reference or control, an assessment of, for example, immune function, responsiveness to therapy and/or cancer survival, can be made. For example, where immune function is being assessed, the reference or control may correlate with a normal immune function or an effective immune function, or an abnormal immune function, an ineffective immune function or an impaired immune function. The immune function of the subject and/or the immune function of the T cells in the sample from the subject can therefore be determined by comparing the DNAM-1 level in the sample to the DNAM-1 level in a second sample for which the level of DNAM-1 has a known correlation with immune function of T cells. For example, in certain embodiments, the levels/amount of DNAM-1 in a subject sample is decreased or reduced as compared to levels/amount in a second sample. Where the second sample is representative of a normal immune function or an effective immune function, the comparatively lower levels/amount of DNAM-1 in the subject sample indicates that the T cells in the subject sample have impaired, abnormal or ineffective immune function, and by extension, the subject has impaired, abnormal or ineffective immune function. In other embodiments, the levels/amount of DNAM-1 in a subject sample is increased or elevated as compared to levels/amount in a second sample. Where the second sample is representative of an impaired, abnormal or ineffective function, the comparatively higher levels/amount of DNAM-1 in the subject sample indicates that the T cells in the subject sample have a normal or effective immune function, and by extension the subject has a normal or effective immune function. Where the second sample is representative of normal or effective immune function, the comparatively higher levels/amount of DNAM-1 in the subject sample can indicate that the T cells in the subject sample have improved or particularly effective immune function, and by extension, the subject has an improved or particularly effective immune function. In further embodiments, the immune function of the subject and/or the immune function of the T cells in the sample from the subject is determined by comparing the DNAM-1 level in the sample to the DNAM-1 level in a second sample for which the level of DNAM-1 has a known correlation with immune function of T cells. For example, in certain embodiments, the levels/amount of DNAM-1 in a subject sample is decreased or reduced as compared to levels/amount in a second sample.

In some embodiments, increased or elevated level or amount refers to an overall increase of about any of 10%, 20%, 30%, 40%, 50%, 60%, 70%, 80%, 90%, 100%, 150%, 200%, 250%, 300%, 400%, 500% or greater, in the level or amount of DNAM-1 detected by standard art known methods such as those described herein, as compared to a reference sample, reference cell, reference tissue, control sample, control cell, or control tissue. In certain embodiments, the elevated amount or level is at least about any of 1.5×, 1.75×, 2×, 3×, 4×, 5×, 6×, 7×, 8×, 9×, 10×, 25×, 50×, 75×, or 100× the expression level/amount of DNAM-1 in a reference sample, reference cell, reference tissue, control sample, control cell, or control tissue.

In other embodiments, reduced level or amount refers to an overall reduction of about any of 10%, 20%, 30%, 40%, 50%, 60%, 70%, 80%, 90%, 95%, 96%, 97%, 98%, 99% or greater, in the level or amount of DNAM-1 detected by standard art known methods such as those described herein, as compared to a reference sample, reference cell, reference tissue, control sample, control cell, or control tissue. In certain embodiments, reduced level or amount refers to the decrease of at least about any of 0.9×, 0.8×, 0.7×, 0.6×, 0.5×, 0.4×, 0.3×, 0.2×, 0.1×, 0.05×, or 0.01× the expression level/amount of DNAM-1 in a reference sample, reference cell, reference tissue, control sample, control cell, or control tissue.

In particular embodiments, the number or percentage of DNAM+ T cells in a population is assessed. “DNAM+ T cells” are T cells expressing detectable levels, or levels over a predetermined level that is considered to represent a “positive” result, of DNAM-1 on their surface. In one embodiment, the number or percentage of DNAM+ CD+ T cells are determined, e.g. the number of DNAM+ CD8+ T cells per total CD8+ T cells in a population (e.g. a population of tumour infiltrating T cells).

The level or amount of DNAM-1, or the number of DNAM+ T cells, in a sample can be analyzed by a number of methodologies, many of which are known in the art and understood by the skilled artisan, including, but not limited to, immunohistochemistry (“IHC”), immunofluorescence (IF), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. In particular embodiments, the surface expression of DNAM-1 on T cells is detected and/or analysed by FACS, IF or IHC. For example, the number or percentage of DNAM-1+ T cells (e.g. the number or percentage of DNAM-1+ CD8+ T cells) in a population is assessed by detecting surface expression of DNAM-1 on the T cells by FACS, IF or IHC. As would be appreciated, in such methods, the biological sample from the subject is contacted with an anti-DNAM-1 antibody that is directly or indirectly labelled (e.g. fluorescently labelled) and the formation of a complex between T cells expressing DNAM-1 on the surface and the antibody is then detected. In further embodiments, the T cells are also labelled, selected or isolated prior to, during or after contact with the anti-DNAM-1 antibody, such as by using an anti-CD3 and/or anti-CD8 antibody.

Where the methods are used to determine the likelihood of responsiveness of a subject with cancer therapy, such as to immune checkpoint blockade, or cancer survival time, the subject may have any cancer. In some examples, the cancer is a solid cancer or tumour. In other examples, the cancer is a leukemia. Non-limiting examples of cancer include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from melanoma, lung cancer, breast cancer, bladder cancer, renal cell carcinoma, liver cancer, head and neck cancer and colorectal cancer.

In some examples, once an assessment of the subject's immune response, cancer survival or responsiveness to cancer therapy is made, the subject is further administered a therapy (e.g. adoptive cell therapy, chemotherapeutic therapy (e.g. immune checkpoint inhibitor therapy), anti-infective therapy, and/or any other therapy described above). For example, if the subject is determined to be likely to respond to cancer therapy (e.g. a chemotherapeutic agent, such as immunotherapy, such as immune checkpoint inhibitor therapy) or to have a particular survival time (any survival time), then the subject may be administered the cancer therapy (e.g. a chemotherapeutic agent, such as immunotherapy, such as immune checkpoint inhibitor therapy). If the subject is determined to be unlikely to respond to cancer therapy or to have an impaired immune function, then the subject may be administered a therapy for enhancing immune function (including T cell function) and/or responsiveness to therapy, such as any described above in section 4. In particular embodiments, the subject is administered a T cell described herein that expresses DNAM-1 (including endogenous, recombinant and/or modified DNAM-1).

In order that the invention may be readily understood and put into practical effect, particular preferred embodiments will now be described by way of the following non-limiting examples.

### EXAMPLES

### Example 1

### Materials and Methods

Mice

Wild-type (WT) C57BL/6 were purchased from Walter and Eliza Hall Institute for Medical Research or bred in house. C57BL6 Pmel-1 TCRtg GFP mice (Glodde et al., 2017), C57BL/6 CD226-deficient (CD226KO) mice (Gilfillan et al., 2008), C57BL/6 CD226KO Pmel-1 TCRtg GFP mice, C57BL/6 CD226Y319F (CD226Y) mice (Zhang et al., 2015), C57BL/6 CD226Y Pmel-1 TCRtg GFP mice and C57BL/6 CD155-deficient (CD155KO) (Li et al., 2018) mice were bred in-house and maintained at the QIMR Berghofer Medical Research Institute. Mice greater than 6 weeks of age were sex-matched to the appropriate models. The number of mice in each group treatment or strain of mice for each experiment is indicated in the figure legends. In all studies, no mice were excluded based on pre-established criteria and randomization was applied immediately prior to treatment in therapy experiments. Experiments were conducted as approved by the QIMR Berghofer Medical Research Institute Animal Ethics Committee.

Cell Lines and Culture

Mouse B16F10 (melanoma) (originally obtained from ATCC), B16F10^{ctrl}, B16F10C^{D155KO} (Li et al., 2018, J Clin Invest 128, 2613-2625), MC38 (colon adenocarcinoma), MC38-OVA^{dim}, MC38-OVA^{hi} (Gilfillan et al., 2008, J Exp Med 205, 2965-2973), HEK293T (kind gift from A/Prof Steven Lane, QIMR Berghofer, Brisbane Australia), and RM-1 (prostate carcinoma) (Blake et al., 2016, Cancer Discov 6, 446-459) cells were grown in Dulbecco's Modified Eagle Medium (DMEM; Gibco) supplemented with 10% Fetal Calf Serum (FCS; Cell Sera), 1% glutamine (Gibco), 1% sodium pyruvate, 1% non-essential amino acids (Gibco), 100 IU/ml Penicillin, and 100 μg/ml Streptomycin (Gibco). Mouse LWT1 (melanoma) (Ferrari de Andrade et al., 2014, Cancer Res 74, 7298-7308) and HCmel12-PmelKO-Tyrp1-Scarlett-hgp100 (HCmel^{12hgp100}) (melanoma) (Effern et al., under review) cells were cultured in “complete RPMI medium” consisting of RPMI-1640 (Gibco) supplemented with 10% FCS (Cell Sera), 1% glutamine (Gibco), 1% sodium pyruvate, 1% non-essential amino acids (Gibco), 100 IU/ml Penicillin, and 100 μg/ml Streptomycin (Gibco). CHO derived cell lines were cultured in CHO complete medium (Thermo Fisher) supplemented with 4% glutamine (Gibco) and 2% Hypoxanthine, Thymidine (Corning). B16F10 and its variants, HCmel^{12hgp100}, RM-1 and LWT1 cell lines were maintained at 37° C., 5% CO2. All MC38-derived cell lines were maintained at 37° C., 10% CO2. CHO derived cell lines were maintained at 37° C., 8% CO2 at 125 rpm. HCmel12 cell lines lacking endogenous gp100 and expressing the human gp100 epitope-tagged to the Tyrosinase related protein 1 locus were generated as described previously (Effern et al., under review). Injection and monitoring procedures were described in previous studies (Glodde et al., 2017, Immunity 47, 789-802 e789; Li et al., 2018, J Clin Invest 128, 2613-2625). All cell lines were routinely tested negative for Mycoplasma, but cell line authentication was not routinely performed.

Primary Cell Cultures

For in vitro studies single cell suspensions of bone marrow cells and/or T cells isolated from spleens of mice were cultured in “complete RPMI medium” (described above) supplemented additionally with 1 mM HEPES (Gibco), and 50 μM 2-mercaptoethanol (Sigma) at 37° C., 5% CO₂.

Generation of CHO-OKT3 and CHO-OKT3-CD155 Overexpressing Cells

Human PVR (NM_006505, NP_006496) was sub-cloned from R&D Systems RDC1289 into pLenti-EF1a (Origene). Lentivirus was produced using the Lenti-X Single Shot packaging system according to the manufacturer's instruction (Clontech). CHO-OKT3 (Subclone 2E5; Immuno-Oncology Discovery, Bristol-Myers Squibb) cells were transduced with polybrene (Sigma; 5 μg/ml) and sorted for CD155 expression.

Generation of CRISPitope-Engineered Cell Lines (Hcmel12^{hgp100})

HCmel12^{hgp100} cell lines were generated as described previously (Effern et al., under review). Briefly, a stable knock-out of the PmeI gene in HCmel12 melanoma cells was generated by targeted CRISPR/Cas9. HCmel12 cells were transfected with px330-U6-Chimeric_BB-CBh-hSPCas9 (Addgene #42230) plasmid encoding a double-stranded DNA oligonucleotide targeting upstream of the genomic region encoding for the pmel-1 T cell epitope in exon 1 of the murine PmeI gene. Genomic aberrations of PmeI-knockout single cell clones were characterized by next generation sequencing and analysed using the web tool OutKnocker (Schmid-Burgk et al., 2014, Genome Res 24, 1719-1723) The plasmid px330-U6-Chimeric_BB-CBh-hSPCas9 was used as target selector. A double-stranded DNA oligonucleotide targeting the C-terminus of the desired target gene was cloned into the Bbsl-digested px330 to generate a functional sgRNA. Frame selectors pCAS9-mCherry-Frame+0, pCAS9-mCherry-Frame+1 and pCAS9-mCherry-Frame+2 were a gift from Veit Hornung (LMU, Munich, Germany; Addgene #66939, #66940 and #66941). Universal donor plasmids were cloned based on the pCRISPaint-mNeon-PuroR plasmid described previously (Schmid-Burgk et al., 2016, Nat Commun 7, 12338). The universal donor pCRISPaint-mNeon-PuroR was a gift from Veit Hornung (LMU, Munich, Germany). Using molecular cloning approaches, the pCRISPaint-mNeon-PuroR plasmid was further modified by (1) exchanging the Puromycin resistance cassette by a Blasticidin resistance cassette, (2) exchanging the Methionine start codon (ATG) of the resistance cassettes by a Glycine (GGG) to prevent transcription from random genomic integrations, (3) exchanging the mNeon fluorescent protein by the mScarlet fluorescent protein, and (4) addition of a FLAG-tag and the human gp100 epitope (aa₂₅₋₃₃) to the fluorescent protein (C-terminus). CRISPitope-engineered HCmel12 melanoma cells were generated by targeting the C-termini of the Pmel gene by CRISPR-assisted insertion of epitopes. For CRISPitope plasmid transfection, 50.000-100.000 HCmel12-gp100 knock out cells were seeded in a 96-well plate and transfected with 200 ng of DNA (50 ng target selector, 50 ng frame selector and 100 ng universal donor) in Opti-MEM I (Life Technologies) using 0.6 μl of Fugene transfection reagent (Promega) according to the manufacturer's instructions. After selection, CRISPitope-engineered cell lines were sorted for mScarlet expression using a FACS Aria III high-speed cell sorter (BD) and subsequently polyclonal cultures of the individual cell lines were established.

Tumor Transplantation

Cohorts of syngeneic C57BL/6 mice were injected subcutaneously (s.c.) with 1×10⁵ B16F10 melanoma, 1×10⁵ or 1×10⁶ MC38 colon adenocarcinoma, 2×10⁵ HCmel12hgp100 (2×105) cells or 1×10⁶ MC38OVA^{dim}, MC38OVA^{bright} or MCA1956 fibrosarcoma cells in 100-200 μl PBS into the hindflanks of mice. Tumor size was measured as indicated and recorded as mean of two perpendicular measurements in millimetres using electronic calipers. Tumor area was calculated in mm² using the equation: A=length×width. Mice with tumors exceeding 150 mm² were sacrificed unless stated otherwise. Experiments were performed in groups of four or more mice.

MCA-Induced Carcinogenesis

For 3-methylcholanthrene (MCA; Sigma) carcinogen-induced fibrosarcoma, male mice of the indicated genotypes were injected subcutaneously with 5 μg or 25 μg MCA in 100 μL sterile corn oil and development of fibrosarcoma was monitored.

Experimental Metastasis

For primary metastases B16F10 melanoma (1×10⁵ cells), LWT1 melanoma (5×10⁵ cells) or RM-1 prostate carcinoma (1×10⁴ cells) were injected intravenously. The metastatic burden was quantified in the lungs after 14 days by counting colonies on the lung surface as described previously (Blake et al., 2016, Cancer Discov 6, 446-459).

Vk*MYC Myeloma Transplant Model

The transplantable Vk*MYC myeloma cell line Vk12598 was maintained and expanded as previously described (Nakamura et al. 2018, Cancer Cell 33, 634-648 e635). Vk12598 MM cells (5×10⁵) were injected i.v. into the tail vein of indicated genotypes of mice. Survival was monitored daily according to institutional ethic guidelines and mice were euthanized when they developed signs of paralysis and reduced mobility.

Immune Checkpoint Blockaid

For MC38-OVA^{dim} tumors, therapeutic blockade of PD-1 was performed on day 10, 14, 18 and 22 after s.c. tumor cell injection by i.p. injections of 250 μg rat anti-mouse PD-1 IgG2a (clone RMP1-14; BioXcell) or rat-control IgG2a mAb (clone 1-1; Leinco) in 100 μl PBS. For B16F10, therapeutic blockade of PD-1 and CTLA-4 was performed on days 6, 9, 12 and 15 after s.c. tumor cell injection using i.p. injections of 250 μg rat anti-mouse PD-1 IgG2a (clone RMP1-14; BioXcell) and 250 μg hamster-anti-mouse CTLA-4 IgG2a (clone UC10-4F10-11; BioXcell) or rat-control IgG2a mAb (clone 1-1; Leinco) resp. control-hamster IgG (BioXcell) in 100 μl PBS.

Adoptive T Cell Immunotherapy

ACT immunotherapy was performed as previously described with slight modifications (Glodde et al., 2017, Immunity 47, 789-802). In brief, when transplanted B16F10 melanomas reached a size of >5 mm in diameter, mice were preconditioned for ACT by a single i.p. injection of 2 mg (100 mg/kg) cyclophosphamide in 100 μl PBS one day before intravenous delivery of 0.5×10⁶gp100-specific CD90.1⁺CD8⁺DNAM-1⁺ or DNAM-1⁻ Pmel-1 T cells (in 200 μl PBS) isolated from spleens of Pmel-1 TCR transgenic mice treated for 2 weeks with anti-CD137 antibody (100 μg i.p. rat-anti-mouse CD137; clone 3H3; BioXcell; in 100 ml PBS, every 3 days). The adoptively transferred T cells were activated in vivo by a single i.p. injection of 5×10⁸ PFU of a recombinant adenoviral vector Ad-gp100 in 100 μl PBS. 50 μg of CpG 1826 (MWG Biotech) and 50 μg of polyinosinic:polycytidylic acid (poly(I:C), Invivogen) in 100 μl saline were injected peritumorally 3, 6, and 9 days after adoptive Pmel-1 T cell transfer.

Tissue Processing

Tumors and peripheral lymphoid tissues were processed using standard protocols. Briefly, tumors or lymphoid organs were harvested from mice and dissociated using GentleMACS Homogenizer (Miltenyi) as per manufacturer's instructions followed by incubation with 1 mg/ml Collagenase D (Sigma) and 1 mg/ml DNaseI (Roche) in “complete RPMI medium” at 37° C. After 30-45 mins tissues were passed through 70 μm cell strainers (Greiner) and further analysed.

Flow Cytometry

Mice were killed and organs were harvested and prepared for flow cytometry as previously described (Gao et al Glodde et al). Single-cell suspensions from various organs were incubated on ice for 15 min in Fc blocking buffer (PBS containing 2% FBS and anti-CD16/32 (clone 2.4G2; hybridoma obtained from ATCC). Reagents or antibodies targeting the following epitopes were purchased from BioLegend: CD3 (145-2C11), CD8 (53-6.7), CD90.1 (OX-7), CD44 (IM7), Vb13 (MR12-3), CD62L (MEL-14), DNAM-1 (10E5), IFN-γ (XMG1.2), TIGIT (1G9), TCRβ (H57-597), TNF (MP6-XT220), Zombie Yellow or Aqua Fixable Viability Kit. Reagents or antibodies targeting the following epitopes were purchased from eBioscience: CD45.2 (104) and TCRP (H57-597). Reagents or antibodies targeting the following epitopes were purchased from BD Biosciences: PD-1 (J43) and Ki67 (B56). OVA-Tetramer (SIINFEKL) was purchased from Prof Andrew Brooks, DMI/PDI/University of Melbourne. For Tetramerization Streptavidin-APC (Biolegend) was added six times every 10-15 minutes until a 1:1.7=Mononner:SA-APC ratio was reached. Assembled Tetramer was used within one week. Tetramer staining was performed for 30′ on ice. Cell number was calculated by using BD Liquid Counting Beads (BD Biosciences). For intracellular cytokine detection, lymphocyte-enriched tumor homogenates were incubated in RPMI-1640 supplemented with 10% FCS, Cell Stimulation Cocktail plus protein transport inhibitors (stimulated cells) (eBioscience), or GolgiStop and GolgiPlug (unstimulated control cells) (both from BD Biosciences) at 37° C. for 4 h. After cell-surface staining, samples were fixed and permeabilized using Intracellular Fixation & Permeabilization Buffer Set (eBioscience), and stained with antibodies in 1× Permeabilization Buffer. For intranuclear staining single-cell suspensions were stained with antibodies against cell-surface antigens as aforementioned, fixed and permeabilized using FoxP3 Fix/Perm Buffer Kit (Biolegend) followed by intranuclear staining. Cells were acquired on the BD LSR Fortessa Flow Cytometer (BD Biosciences), CytoFLEX (Beckman Coulter) or Cytek Aurora (3 lasers) Flow Cytometer (Cytek). Analysis was carried out using FlowJo V10 software (FlowJo, LLC). tSNE analysis of concatenated samples was performed in FlowJoV10.2 after appropriate down sampling to the indicated number and R based “tSNE plots” script was used for visualisation.

Cell Sorting

DNAM-1⁺ or DNAM-1⁻ pnnel-1 T cells have been isolated from spleens of pmel-1 TCR transgenic mice after 2 weeks of anti-CD137 treatment (100 μg i.p every three days) stained with antibodies against CD8, CD90.1 and DNAM-1 and purified using a BD FACSAria II Cell Sorter (BD Biosciences).

Mouse T Cell Stimulation Assays

For mouse T-cell activation, splenocytes or when indicated MACS separated CD8⁺ T-cells were activated in flat-bottom 96-well plates with plate-bound anti-CD3 (clone 145-2C11; Biolegend; 1-2-5 μg/ml, 50-100-250 ng/well) plus soluble anti-CD28 (clone 37.51; Biolegend; 1-2 μg/ml) at 0.5-2×10⁵ cells/ml. In some experiments, plate-bound CD155-Fc (Sino Biological) or irrelevant human IgG1 (BioXCell) at 0.3 μg/well were present. Plate binding of antibodies/proteins was performed in PBS overnight at four degrees and wells were washed with PBS immediately before the experiment.

CD226 Internalisation

CD8⁺ T-cells of the indicated genotype were isolated using MACS technology

(Miltenyi) according to manufacturer's protocol from single-cell suspensions of spleens. After isolation, cells were stimulated with soluble anti-CD3 (clone 145-2C11; Biolegend; 1 μg/ml) and soluble anti-CD28 (clone 37.51; Biolegend; 1 μg/ml) antibodies in “complete RPMI medium” supplemented with 20 IU/ml human IL-2 (Novartis) for 48 h at a concentration of 1-2×10⁶ cells/ml. Cells were then seeded in a flat-bottom 96-well plate which was coated overnight at 4° with hIgG1 (IgG; BioXcell; 0.3 μg/well) or recombinant mCD155 fused to the carboxy-terminal Fc region of human IgG1 (CD155-Fc; Sino Biological; 0.3 μg/well). After 1 h incubation at 37° C., 5% CO₂ cells were harvested and surface stained for CD8⁺ (BV421; clone 53-6.7; Biolegend) and CD226 (AF647; clone 10E5; Biolegend) followed by fixation, permeabilisation, and intracellular stain of CD226 (PE; clone 480.1; Biolegend). Cells were then immediately acquired on a four laser, 12 channel Amnis ImageStream^{X} MkII (Amnis, EMD Millipore, Seattle, Wash., USA) at a ×60 fold magnification at low speed. Data analysis was performed using IDEAS software (Amnis). The gating strategy for analysis involves the selection of cells in focus based on “gradient RMS”. Cells with high “Aspect ratio” and low “Area” values were selected as they are likely singlets and subjugated on CD8⁺ (BV421) cells. Finally, good quality, focused and centered cells were selected and at least 100 cells per group were analysed.

Analyses of Immunological Synapse Formation

Synapse formation assay was performed as previously described (Markey et al., 2015, J Immunol Methods 423, 40-44) with minor modifications. Bone marrow derived dendritic cells were prepared by flushing the long bones from the hind legs of sacrificed mice of the indicated genotype. Cells were seeded at 1-3×10⁶ cells/ml in “complete RPMI medium” supplemented with 1 ng/ml mouse GM-CSF. After three-four days non-adherent cells were collected and further cultured. Experiments were performed after at least seven days of in vitro differentiation. The day before the assay BMDC were harvested and labeled with Cell Trace Violet (CTV; Life Technologies) as per the manufacturer's instructions and overnight peptide-loaded with 1 μg/ml H2-D^{b} binding peptide hgp100₂₅₋₃₃ peptide (KVPRNQDWL; Mimotopes). On the same day, CD8⁺ T-cells of the indicated genotype were isolated using MACS technology (Miltenyi) according to the manufacturer's instruction from single-cell suspensions of spleens and plated at 1×10⁶ cells/ml in 10-20 U hIL-2 (Novartis). The next day, T-cells were harvested and CFSE (Biolegend) labeled as per the manufacturer's protocol. BMDC and T-cells were then co-cultured at a 1:2=T:DC ratio for 1 h at 37° C., 5% CO₂. At the end of incubation period cells were fixed with 3× volume 1.5% Paraformaldehyde at room temperature, followed by surface stain of LFA-1 (PE-Cy7; clone H155-78; Biolegend) in the presence of anti-CD16/CD32 (clone 2.4G2; produced in-house) in PBS containing 2% (v/v) FCS (Cell Sera). Following surface staining, cells were washed and permeabilized using 100 μL of 0.1% Triton-X (Sigma). 3 μL of phalloidin (AlexaFluor 647; Life Technologies) was added to each sample, and cells were incubated for 30 minutes at room temperature. During the whole staining process, cells were treated extremely carefully and vortexing, thorough resuspending, or EDTA was deliberately avoided to maintain established synapse formations. At the end of the staining period, cells were washed and immediately acquired on a four laser, 12 channel Amnis InnageStream^{X}MkII (Amnis) at a ×60 fold magnification at low speed. Data analysis was performed using IDEAS software (Amnis). The gating strategy for analysis involves the selection of cells in focus based on “gradient RMS”. Cells with intermediate “Aspect ratio” and intermediate “Area” values were selected as they are likely doublets. We subgated on double-positive CTV⁺ and CFSE⁺ “events”. Finally, good quality, focused and centered were selected and at least 40 synapses per group were analysed. The interface mask was then applied and the T-cell defined as the target of interest. The fluorescence intensity of LFA-1 and Phalloidin within the Interface mask serves as a surrogate marker for the strength and intensity of the immunological synapse. Statistical significance was determined using a non-parametric one-way ANOVA.

Retroviral Transduction of Mouse T Cells

Full-length mouse CD226 cDNA sequence was synthesized and separately cloned (BioMatik) into the MSCV-IRES-GFP plasmid (kind gift from A/Prof Steven Lane, QIMR Berghofer, Brisbane, Australia; Addgene #20672). For generation of retrovirus, HEK293T cells were plated on 10 cm dishes overnight at a concentration of 4×10⁶ cells/dish. The following day, packaging plasmid pCL-Eco (kind gift from A/Prof Steven Lane, QIMR Berghofer, Brisbane, Australia) and plasmid encoding either MSCV-IRES-GFP-Mock or MSCV-IRES-GFP-CD226 WT-full-length were mixed along with Fugene 6 (Promega) as per the manufacturer's protocol at a 3:1=Fugene:DNA ratio and applied to the HEK293T cells overnight. Medium was then replaced and viral supernatant was collected twice in the following 48 h. Retroviral supernatants were spun for 2-6 h at 17.000 g for virus concentration and immediately stored at −80° C. For transduction, CD8⁺ T-cells were plated at 1-3×10⁶ per well in 6-well plates that had been coated overnight in 5 μg/ml Retronectin (Takara Bio Inc.) and viral supernatant in a 1:1 vol/vol ratio and 4 μg/ml Polybrene (Sigma) was added. Spinfection was performed at 30° C. for 2 h at 2000 g with no acceleration or brake. Media was replaced after 2-4 h. In some experiments, spinfection was repeated after 24 h. Cells were maintained in 100 IU/ml human IL-2 (Novartis) and 2 ng/ml mouse IL-7 (Biolegend) and checked for purity until used in experiments and ACT.

Cytokine Bead Array

Tumor single-cell suspensions were resuspended in an equal volume of “complete RPMI medium” and incubated for 4-5 h at 37° C., 5% CO₂ followed by supernatant collection. The supernatant was stored at −80° C. until analysis using Cytokine Bead Array (BD) using manufacturer's protocol.

Human T Cell Stimulation Assays

PBMCs were thawed and treated with DNAse I (Roche) to remove dead cells prior to culture. 1×10⁵ PBMC were cultured in RPMI-1640 (Gibco)+10% FCS (Cell Sera) in 200 μl volume in U-bottom 96-well plates. T-cell activation was achieved by the addition of 2×10⁵ CD3/CD28 stimulator beads (Thermo Fisher Scientific). The culture was incubated at 37° C., 5% C0₂. At the completion of the culture period, the cells were stained for surface markers and analysed by flow cytometry.

Human CD226-CD155 Interaction Using Artificial APC

Ficoll processed and enriched CD3⁺ T cells using RosetteSep (Stemcell) from human healthy blood were plated at 1×10⁵ cells per well into U-bottom 96-well plates. 5×10⁴ of OKT3 single expressing or OKT3 and CD155 dual expressing CHO cells were used to present CD155 to human T cells in vitro. Co-cultured T cells were harvested and fixed using 2% PFA in PBS at each time point. For pre-activation of CD8⁺ T cells, prepared T cells were cultured for 7 to 10 days in “complete RPMI medium” supplemented with 25 μL/ml of anti-CD3/CD28 tetrametric antibody (Stemcell) and 80 IU/ml of human IL-2 (PeproTech). For CD155 blockade, titrated anti-human CD155 antibody (clone SKII.4, Biolegend) were pre-incubated with CHO cells at indicated dosage for 30 minutes prior to co-culture with human T cells.

Patients and Specimens

All procedures involving human participants had approval from both the QIMR Berghofer Medical Research Institute Human Research Ethics Committee (HREC) (EC00278) and Royal Brisbane and Women's Hospital HREC (EC00172) and this study conformed to the Declaration of Helsinki. All tissue and blood samples were collected after obtaining written informed consent in accordance with participating hospitals/research institute Human Research Ethics Committee procedures and guidelines.

HNSCC Specimens

HNSCC specimens were received from the Metro North HHS, Royal Brisbane and Women's Hospital, Brisbane, Australia. Fresh samples were processed using a commercially available Tumor Dissociation Kit (Miltenyi) including a tissue disaggregation platform (GentleMACS, Miltenyi) both according to the manufacturer's protocol. Peripheral blood mononuclear cells (PBMC) were isolated by Ficoll density gradient centrifugation from blood samples taken from the patient at the time of tumor excision. PBMC and tumor single-cell suspensions were then cryopreserved until further usage. Cryopreserved samples were thawed and incubated in RPMI 1640 containing 10 pg/ml DNAse I (Sigma) and incubated at 37° C. for 1 h to eliminate clumping and debris. The T-cell stimuli used were 5 μl of anti-CD3 and anti-CD28 microbeads (Dyna beads, ThermoFisher Scientific) at approximately 2×10⁵ cells/well in RPMI 1640 (Gibco) plus 10% FCS (Cell Sera), cultured at 37° C., 5% CO₂ for 4 h prior to staining for flow cytometry.

Melanoma Specimens

Archival formalin-fixed paraffin-embedded (FFPE) tissue specimens were obtained from pre-treatment patients with radiologically confirmed non-lymphoid stage IV melanoma (AJCC) from the Melanoma Institute Australia (MIA) as tissue microarrays from tumor core and tumor margins. Patient demographics and immunotherapeutic interventions are listed in Table 2. All patients received PD1 based immunotherapy between January 2015 and May 2018 and had provided written informed consent for the use of samples according to the institutional regulations. Pathology reports from all patients treated with immunotherapy were reviewed. Cases were selected for inclusion if there was sufficient archival FFPE tissue and clinical annotation for analysis.

TABLE 2

| | melanoma MIA (n = 31) | |
|---|---|---|
| | Responder | Non-responder |
| # patients | 18 | 13 |
| Sex | | |
| male | 13 | 9 |
| female | 4 | 5 |
| Age | | |
| Median | 69 | 55 |
| Range | 47-82 | 40-85 |
| LDH | 3 | 5 |
| 1st line IO | 14 | 9 |
| Therapy details | | |
| Nivo | 1 | 0 |
| Pembro | 2 | 1 |
| Ipi + Nivo | 1 | 2 |
| Ipi + Pembro | 10 | 6 |
| RECIST category | | |
| CR | 6 | 0 |
| PR | 11 | 3 |
| SD | 1 | 4 |
| PD | 0 | 6 |

LDH = Lactate dehydrogenase, ICB = Immuncheckpoint blockade, Nivo = Nivolumab; Pembro = Pembrolizumab, Ipi = Ipilimumab, CR = complete response; PR = partial response, SD = stable disease, PD = progressive disease.

Immunohistochemical Staining for Human CD155

The TMA was sectioned at 3 μm on superfrost+ glass slides and stored under vacuum until INC was performed. Slides were dehydrated at 65° C. for 20 min, deparaffinized in xylene and rehydrated in graded ethanol. Antigen retrieval was performed in EDTA buffer (pH 9) in a Decloaking Chamber (Biocare Medical) at 100° C. for 20 min. IHC was performed on an Autostainer-Plus (DAKO). The primary rabbit anti-human antibody against CD155 (D3G7H; CST) was incubated for 45 minutes at room temperature using a 1:100 dilution and visualized using the MACH3 Rabbit HRP polymer detection system (Biocare) and DAB Chromogen Kit (Biocare) as per the manufacturer's instructions. Slides were counterstained with diluted hematoxylin. CD155 was then evaluated as the percentage of membrane positive tumor cells and the maximum intensity of the immunohistochemical signal was recorded. CD155 expression was assigned using the H-score method and categorized as follows; low (0-99) or high (200-300).

Multiplex Immunohistofluorescence (mIHF) Staining

Using above mentioned TMA and multispectral fluorescence imaging, we quantified the expression of CD226 on CD8⁺ T cell in melanoma samples. SOX10 was used to identify melanoma cells and DAPI was used as a nuclear stain. Specimens were sectioned at 4 μm onto superfrost+ microscope slides and stored under vacuum until mIHF was performed. Heat-induced antigen retrieval with EDTA target retrieval buffer (DAKO) was performed using an antigen-decloaker at 100° C. for 20 minutes and washed in Tris-buffered saline solution with Tween 20 (TBS-T) (pH 7.6). Staining was run on an automated tissue stainer (DAKO). Primary antibodies were visualized using the OPAL multiplex TSA detection system (PerkinElmer) as per the manufacturer's instructions with heating for 20 minutes at 100° C. using EDTA buffer between sequential staining rounds to strip prior bound antibody/HRP complexes. Primary antibodies, were diluted in Van Gogh Yellow Diluent (Biocare) and incubated for 30 minutes, followed by a two-step polymer-HRP detection system (Biocare) and then labelled with TSA-based fluorophores (Opal Reagent Pack; PerkinElmer). The following primary antibodies/clones were used sequentially in the order listed with antibody dilutions and Opal-fluorophores listed in parenthesis: CD8/144b (1:1000; Opal570), CD226/102 (1:500; Opal520) and SOX10/BC34 (1:500; Opal690).

Multiplex-IHF Image Acquisition and Analysis

Fluorescence-stained slides were scanned using a Vectra imaging system (PerkinElmer). Whole slide scanning was done at 4× magnification using mixed fluorescence followed by 20× multispectral imaging. Images were spectrally unmixed followed by tissue and cell segmentation using Inform analysis software (PerkinElmer; v2.2.1). Merged data files were processed, and fluorescence thresholds were set using Spotfire image-mapping tools (Tibco Spotfire Analyst; v7.6.1) followed by segmented cell counting using Spotfire. Stratification of patients into “high CD226⁺CD8⁺/CD8⁺” versus “low CD226⁺CD8⁺/CD8⁺” or “high CD8+” versus “low CD8⁺” was determined using the “Cutoff Finder” tool (Budczies et al., 2012, PLoS One 7, e51862).

Gene Expression Analysis of Human PBMCs

Isolated PBMCs from healthy donors were plated onto 48-well plate at 0.5×10⁶ cells/well. The cells were stimulated for 48 hours with or without suboptimal concentration of anti-CD3 (clone OKT3; BioLegend, 200 pg/ml) in the presence of isotype control (MOPC-21 mouse IgG1; BioLegend; 1 μg/ml) or anti-CD226 (clone DX11; BioLegend; 1 μg/ml). Stimulated cells were harvested and resuspended in 350 μL of RLT lysis buffer (Qiagen). The lysated cells were frozen down immediately and sent to Core Diagnostics (Hayward, Calif.) for Nanostring analysis.

The Cancer Genome Atlas (TCGA) Transcriptomic Analysis

Gene expression data (RNA-seq) of TGCA cancer cohorts was accessed and analysed through the cBioportal for Cancer Genomics (http://www.cbioportal.org) using the R-based packages CGDS-R and TCGAbiolinks. Guidelines for the use of TCGA data (https://cancergenome.nih.gov/) were followed. We retrieved individual gene expression values for the genes of interest as RPKM normalized read counts.

Moving Average Analysis

Moving average analysis was performed as previously published (Glodde et al., 2017, Immunity 47, 789-802 e789; Riesenberg et al., 2015, Nat Commun 6, 8755). RPKM-values less than 1 were set to 1 to avoid negative expression values upon log 2-transformation. Samples were ordered by increasing expression values of averaged CD226. The moving average of CD8B, NCAM, IFNG, PVR, GZMB and NECTIN2 gene expression in tumor tissues was calculated using a sample window size of n=20 and respective coloured trend lines were added to the bar plots. Significance of non-parametric Spearman rank correlation was determined by an asymptotic Spearman correlation test using the original log 2 expression values.

Statistical Analysis

Statistical analysis were determined with Graph Pad Prism 7 and 8 (GraphPad Software). If not stated otherwise, Student's t-test was used for comparisons of 2 groups, One-way ANOVA for comparison of multiple groups with posthoc Tukey's test for multiple comparisons. Significance of in vivo experiments was calculated by log-rank (Mantel-Cox) test for Kaplan-Meier survival analysis or Two-way ANOVA with posthoc Tukey's test for multiple comparisons. A Fisher's exact test was used to determine the significance of the proportion of tumor free mice. Differences between groups are shown as the mean±SD. P values of less than 0.05 were considered statistically significant. p<0.05=*; p<0.01=**; p<0.001=***; p<0.0001=****.

### Example 2

### CD8 T Cell-Mediated Responses in DNAM-1 Deficient Mice

As opposed to most other activating receptors, DNAM-1 (or CD226) is homogeneously expressed on naïve (TN) and central memory (TCM) CD8+ T cells in mice, while only a small proportion of effector memory (TEM) CD8+ T cells were found to be DNAM-1 negative (data not shown). However, upon T cell receptor (TCR) stimulation of splenic CD8+ T cells, CD226 is uniformly upregulated (data not shown).

CD8+ T cell-mediated responses were assessed in wild-type (C57BL/6J) and DNAM-1 deficient (alternatively referred to as DNAM-1^{KO} or CD226^{KO}) mice. Mice were injected subcutaneously into the hindflanks with B16F10, MC38, MC38OVA^{bright}, or MC38OVA^{bright} cells as described in Example 1, and the size of the tumor assessed over 15-25 days. Mice were subsequently euthanised and flow cytometry performed to detect total CD8+ and OVA-specific CD8+ T cells infiltrating the tumors.

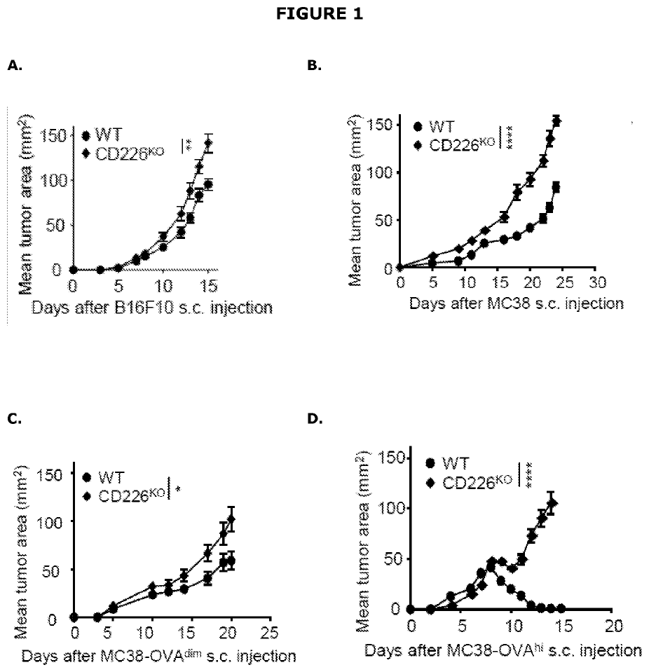

As shown in FIG. 1, CD226^{KO} mice were significantly more susceptible to tumor progression than wild-type mice. Upon analysis of the cells in the MC38OVA^{brigh} tumor, it was observed that the accelerated tumor growth in the CD226^{KO} mice was associated with a reduced percentage of tumor infiltrating CD8+ T cells compared to that observed in wild-type mice (data not shown). Moreover, a lower frequency of IFN-γ-producing CD8+ T cells were observed in CD226^{KO} mice compared to wild-type mice. These data indicate that CD8+ T cell-mediated anti-tumor responses are impaired in DNAM-1 deficient mice.

### Example 3

### Assessment of DNAM-1-T Cell Function in Tumor Microenvironment

To further assess CD226⁻ (i.e. DNAM⁻) T cell function in the tumor microenvironment, CD226 positive (CD226⁺) and CD226 negative (CD226⁻) CD8+ T cells in MC38-OVA^{hi} C57BL/6J (WT) mice were examined. Flow cytometric analyses of CD226 expression on CD8+ T cells infiltrating MC38-OVA^{hi} tumors in WT mice indicated that CD226⁻ CD8+ T cells accumulated in tumors (data not shown). Within this population however, the frequency of IFN-γ-producing cells was significantly lower compared to CD2261⁺ CD8+ T cells (data not showm). Moreover, the frequency of Ki67⁺ cells amongst the CD226⁻ CD8+ T cells was also reduced compared to CD226⁺ CD8+ T cells, indicating that the CD226⁻ CD8+ T cells as a population were less proliferative than CD226⁺ CD8+ T cells. These data indicate that dysfunctional CD226⁻ CD8+ T cells accumulate in the tumor microenvironment.

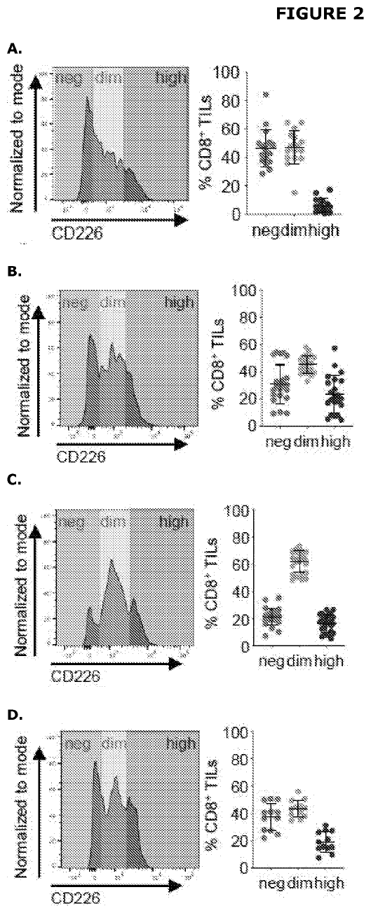

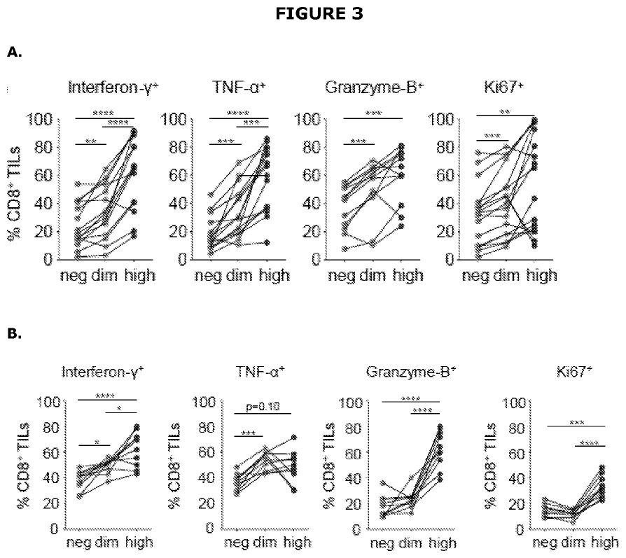

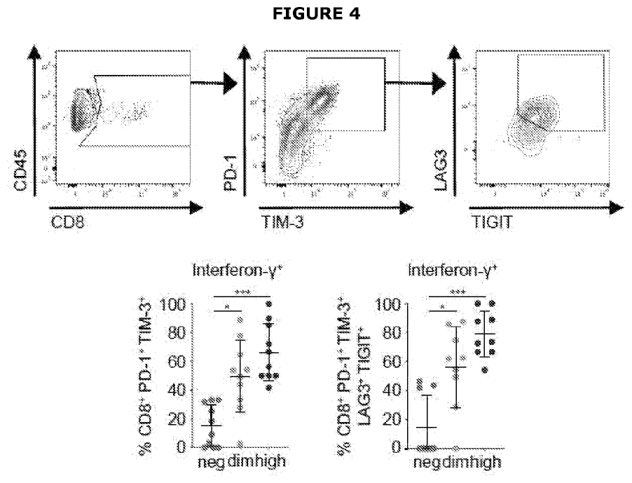

In a further study, the inventors analysed tumor infiltrating CD8⁺ T cells in WT mice by flow cytometry and revealed that T cells can be subdivided into three subsets based on their CD226 expression. A high proportion of tumor-infiltrating CD8⁺ T cells were CD226 negative (CD226^{neg}), a second subset expressed intermediate levels of CD226, similar to resting T cells (CD226^{dim}), and a third subset expressed high levels of CD226 (CD226^{hi}) similar to in vitro activated T cells (FIG. 2). Given that CD226 functions as an activating receptor, it was hypothesized that CD226 surface expression correlates with T cell effector function. Strikingly, it was found that there existed a significant association between CD226 surface expression and the capability of ex vivo restimulated CD8⁺ T cells isolated from B16F10 or MC38 tumors to produce effector cytokines, granzyme B, and to proliferate as indicated by Ki67 staining (FIG. 3). Since CD226^{neg} T cells were found to be dysfunctional, expression of CD226 and inhibitory immune receptors CD8⁺ tumor-infiltrating lymphocytes (TILs) isolated from B16F10 melanoma was assessed. Inhibitory immune receptors are upregulated upon T cell activation and associated with loss of effector function (Thommen and Schumacher, 2018; Wherry and Kurachi, 2015). However, the identification of dysfunctional T cells merely based on the expression of multiple inhibitory receptors is insufficient. In mice, no stringent association between CD226 expression and inhibitory receptors (PD-1, CD96, LAG3, TIGIT, TIM-3) was observed (data not shown). Interestingly, the CD226^{neg} subsets in PD-1⁺Tim-3⁺ or in PD-1⁺Tim-3⁺LAG3⁺TIGIT⁺CD8⁺ T cells were by far the least capable of producing IFN-γ. In contrast, CD226^{hi} T cells were still able to produce large amounts of IFN-γ upon ex vivo re-stimulation, despite the presence of multiple inhibitory immune receptors (FIG. 4). Thus, the data suggest that CD226 is a more specific marker to define functionality of T cells in the TME compared with the expression of multiple inhibitory immune receptors.

### Example 4

### Assessment of the Importance of Tyrosine 319 to T Cell Function

As demonstrated in Examples 2 and 3, DNAM-1 is a critical receptor on T cells in anti-tumor immunity. However, by which mechanism DNAM-1 is facilitating a cytotoxic response was unclear. Tyrosine 319 in mouse DNAM-1 (corresponding to tyrosine 322 in human DNAM-1) is absolutely required for NK cell function in vitro and in vivo. To investigate the importance of this residue in T cells, the DNAM-1^{KI} mouse (also referred to as the CD266^{Y} mouse), which expresses DNAM-1 (i.e. CD226) comprising the Y319F mutation, was used.

Preliminary studies demonstrated that this mutation did not affect immune cell development as no significant differences in various lymphocyte populations were observed in healthy CD226^{Y} compared to WT mice (data not shown). Secondly, consistent with the previously published in vitro findings in NK cells (Zhang et al., 2015), CD226^{Y} mice showed a higher susceptibility to NK cell dependent methylcholanthrene (MCA)-induced carcinogenesis and experimental metastasis compared to WT controls (data not shown). Of note, global CD226^{KO} mice had significantly impaired tumor control compared to CD226^{Y} mice, suggesting that Y319 phosphorylation of CD226 only partially contributes to NK cell-mediated tumor immunity in vivo.

Based on these findings, it was hypothesized that Y319 signaling is at least partially required for T cell-mediated tumor immunity. To test this hypothesis, a first study was performed in which MC38-OVA^{dim} tumors (a MC38-variant which expresses intermediate levels of the foreign antigen Ovalbumin) were transplanted into WT, CD226^{KO} (DNAM^{KO}) and CD226^{Y} (DNAM^{KI}) mice. Surprisingly, it was observed that MC38-OVA^{dim} tumors grew significantly slower, and some tumors were completely rejected, in CD226^{Y} mice, whereas all tumors grew progressively in WT and faster in CD226^{KO} mice (data not shown). Flow cytometric analyses revealed lower frequencies of OVA-specific DNAM-1⁻CD8+ T cells in CD226^{Y} mice compared to WT mice (data not shown), and similar frequencies of Ki67⁺ CD8+ T cells and IFN-γ-producing CD8+ T cells compared to WT mice (data not shown). The total number of tumor infiltrating CD8+ OVA-specific T cells in WT, CD226^{KO} and CD226^{Y} mice was similar, and, importantly, the frequency of DNAM-1-negative cells was significantly lower in CD226^{Y} compared to WT mice (˜22% in WT vs. ˜5% in CD226^{Y} mice).

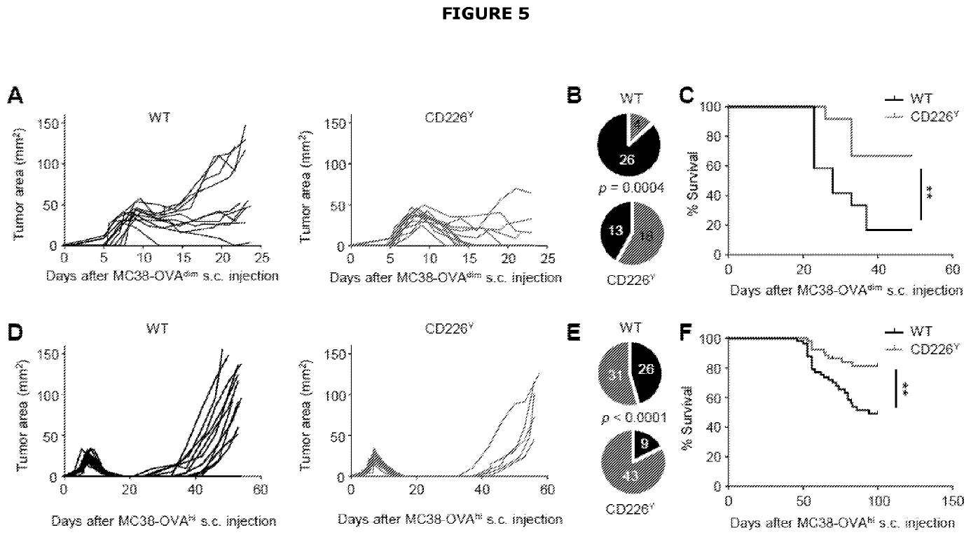

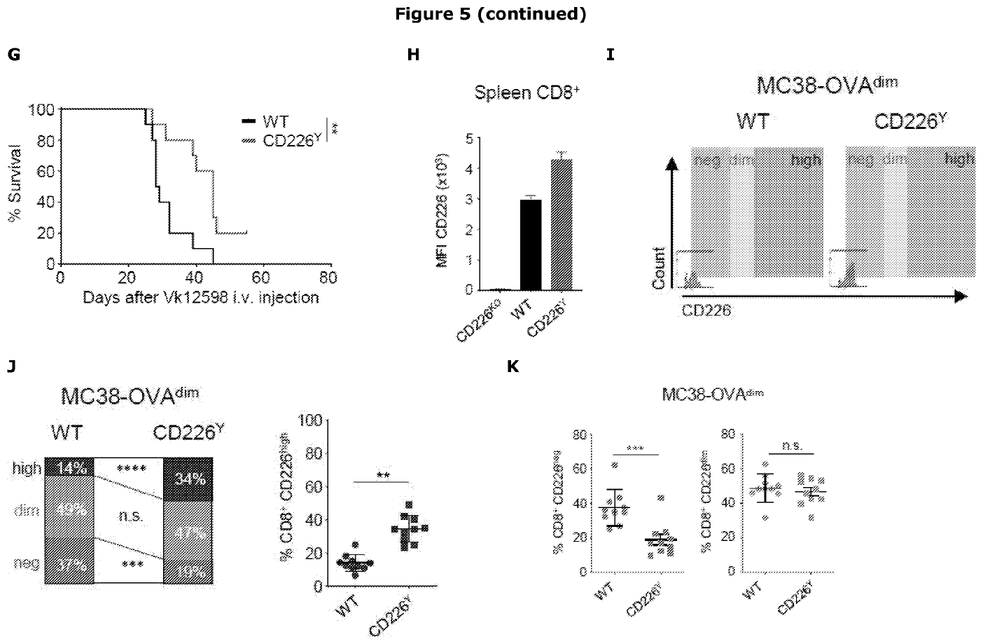

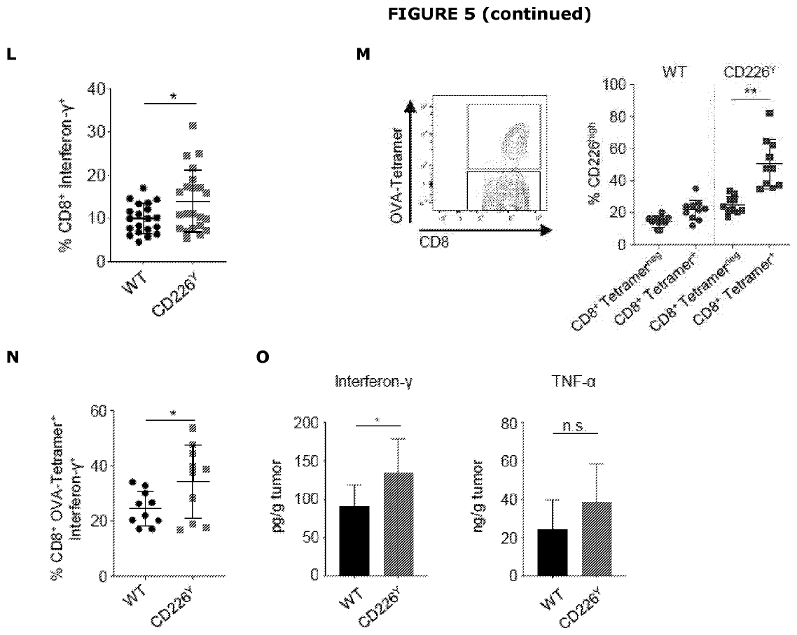

In a subsequent study, immunogenic variants of MC38-OVA^{dim} or high levels (MC38-OVA^{hi}) of the prototypic T cell antigen ovalbumin (OVA) into WT or CD226^{Y} mice. Significantly reduced tumor growth and higher rates of tumor rejection were observed in MC38-OVA^{dim} bearing CD226^{Y} mice compared to WT mice resulting in significantly prolonged survival of CD226^{Y} mice (FIG. 5A-C). When MC38-OVA^{hi} tumors were injected into cohorts of WT and CD226^{Y} mice, initially no difference was observed as all tumors were rejected in both groups. However, presumably due to antigen-loss, tumors frequently relapsed in WT mice (26/57), but recurrence was significantly reduced in CD226^{Y} mice with only 9 of 52 mice relapsing (FIG. 5D, E). Accordingly, a significantly prolonged survival of CD226^{Y} mice was observed (FIG. 5F). These findings from solid tumors were corroborated in a haematological cancer model. CD226^{Y} mice injected with VK12598 multiple myeloma cells also showed improved tumor control and prolonged survival compared to WT mice (FIG. 5G).

Interestingly, in naïve CD226^{Y} mice, a slight increase in CD226 expression in splenic CD8⁺ T cells was observed (FIG. 5H). To understand why a mutation in Y319 led to improved tumor control, the phenotype of tumor infiltrating CD8⁺ T cells was assessed. Significantly higher frequencies of CD22^{hi} CD8⁺ T cells and conversely reduced frequencies of CD226^{neg} CD8+ T cells infiltrating tumors were found in CD226^{Y} mice compared to WT mice (FIG. 5I-K). Interestingly, higher frequencies of IFN-γ (FIG. 5L), and to a lesser extent TNF-α (data not shown), producing TILs isolated from tumors of CD226^{Y} mice were detected. As the tumor cells expressed ovalbumin, CD226 surface expression and effector function in OVA-specific (Tetramer⁺) and non-specific (Tetramer^{neg}) CD8⁺ TILs were assessed. CD226 surface expression was comparable between both T cell subsets in WT mice. Importantly, OVA-specific Tetramer⁺ T cells showed significantly increased CD226 surface expression and increased amounts of IFN-γ compared to Tetramer^{neg} isolated from CD226^{Y} mice (FIG. 5M, N). Accordingly, higher amounts of IFN-γ and TNF-α in the TME of CD226^{Y} mice were observed (FIG. 5O).

Based on this, it is concluded that the mutation of Y319 leads to the retention of DNAM-1 (i.e. CD226) surface expression. These data further indicate that the Y319F mutation does not impair T cell function, as seen in NK cells, but rather improves anti-tumor properties of CD8+ T cells.

Increased CD226 surface expression could enhance adhesion and improve immunological synapse formation of T cells leading to superior cytokine production. While, in human CD4⁺ T cells CD226 signaling through S329 was shown to be important for synapse formation, little is known about the relevance of signaling through Y319 in T cells. Thus, we assessed synapse-quality of antigen-specific CD8⁺ T cells with bone marrow-derived dendritic cells (BMDC) by flow-microscopic quantification of LFA-1 and phalloidin intensity using the ImageStream system. For this, Pmel-1 TCR transgenic mice were crossed to CD226^{Y} or CD226^{KO} mice (herein referred to as WT.Pmel-1, CD226^{Y}.Pmel-1 and CD226^{KO}.Pmel-1), and incubated CFSE-labelled MACS enriched CD8⁺ T cells with CTV-labelled, hgp100₂₅₋₃₃ peptide-pulsed WT BMDCs. Following 1 h of incubation, the intensity of LFA-1 and phalloidin staining at the interface of T cell-BMDC doublets was determined. CD226^{KO}.Pmel-1 T cells clearly showed impaired synapse quality, whereas the synapse quality of CD226^{Y}.Pmel-1 T cells was similar to WT.Pmel-1 T cells (data not shown). This finding suggested that loss of CD226 surface expression, but not the Y phosphorylation site, reduces the ability of T cells to form high-quality synapses. Thus, loss of CD226 surface expression may contribute to impaired effector functions of tumor infiltrating T cells. In summary, mice harbouring a point mutation abrogating CD226 signaling through Y319 have superior anti-tumor immunity which was associated with increased CD226 expression and effector cytokine production in CD8⁺ TILs.

### Example 5

### Tumor Cell CD155 Interaction with DNAM-1

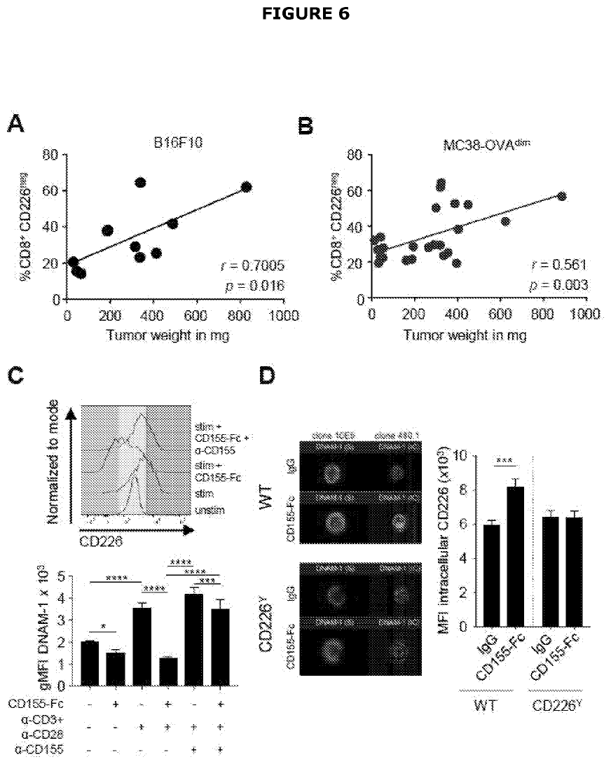

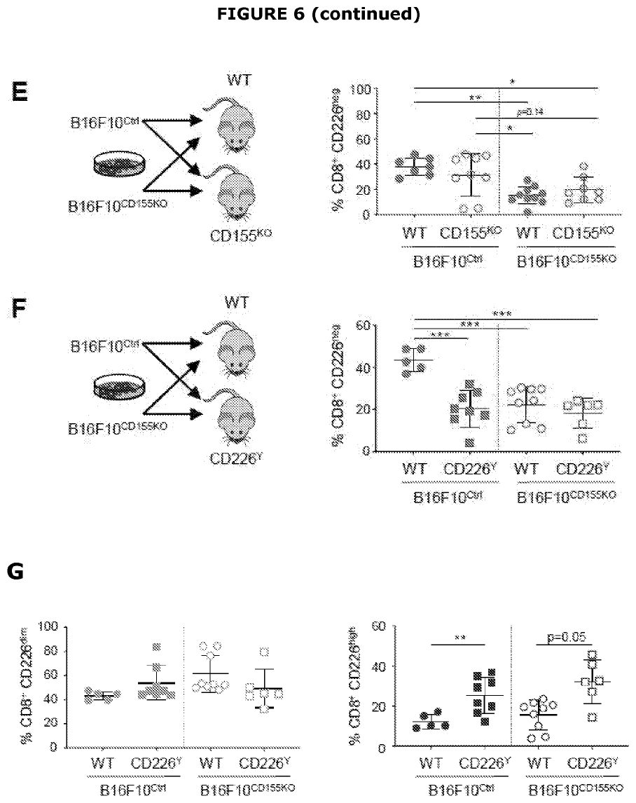

Immune cells isolated from naïve CD155-deficient mice have slightly elevated DNAM-1 (i.e. CD226) expression. As a significant correlation between the frequency of CD226^{neg} T cells and tumor weight (FIG. 6A, B) with both tumor models expressing high levels of CD155 (Li et al. 2018, J Clin Invest 128, 2613-2625), it was hypothesized that loss of CD226 surface expression could be mediated by tumor cell-derived CD155. While in unstimulated T cells in vitro CD155-Fc slightly reduced CD226 expression, it completely prevented TCR-induced upregulation of CD226 (FIG. 6C). Using the ImageStream system, surface and intracellular CD226 levels in pre-activated T cells in the presence of CD155-Fc or control IgG were quantified (FIG. 6D). In brief, surface CD226 was stained with an AF647-conjugated antibody (clone 10E5) followed by intracellular staining of CD226 with a PE-conjugated antibody (clone 480.1). Of note, clone 10E5 blocks the binding of 480.1, but not vice versa, allowing specific assessment of the intracellular fraction of CD226. Indeed, CD155 ligation with CD226 significantly increased the MFI of intracellular CD226 compared to control IgG (FIG. 6D). This suggested an active internalisation process following CD226-CD155 interaction. To validate the finding that CD155 drives CD226 downregulation in vivo, we injected CD155-expressing (B16F10^{ctrl}) or -deficient (B16F10^{CD155KO}) B16F10 melanoma cells into either WT or CD155-deficient (CD155^{KO}) mice (FIG. 6E). This experimental setting facilitated dissection of the importance of tumor cell versus host cell CD155 for the downregulation of CD226 surface expression in tumor infiltrating CD8⁺ T cells. Interestingly, the frequency of CD226^{neg} T cells infiltrating B16F10^{CD155KO} melanoma was significantly reduced in both WT and CD155^{KO} mice compared to B16F10^{ctrl} melanoma (FIG. 6E). In concert, significantly higher frequencies of CD226^{hi} T cells were observed in WT and CD155^{KO} mice bearing B16F10^{CD155KO} melanoma (data not shown). This data supports the idea that high levels of CD155 in the TME contribute to CD226 downregulation in T cells.

Given that tumor infiltrating CD8⁺ T cells in CD226^{Y} mice showed increased CD226 expression (see FIG. 5), it was hypothesized that signaling through Y319 could be important for CD155-mediated internalisation of CD226. To test this in vivo, B16F10^{ctrl} or B16F10^{CD155KO} melanoma cells were injected into WT or CD226^{Y} mice (FIG. 6F). Consistent with the previous findings, the frequencies of CD226^{neg} T cells infiltrating B16F10^{ctrl} tumors in CD226^{Y} mice or infiltrating B16F10^{CD155KO} in WT mice were significantly reduced, while the frequencies of CD226^{hi} T cells were increased (FIG. 6F). Interestingly, fewer CD226^{neg} cells in CD226^{Y} mice bearing B16F10^{CD155KO} melanoma were not observed, suggesting that CD155 triggers downregulation of CD226 through Y319. Of note, CD226^{neg} T cells infiltrating CD155-deficient tumors were still found, thus additional mechanisms seem to contribute to CD226 downregulation.

### Example 6

### Effect of T Cell DNAM-1 Expression on Efficacy of Adoptive Cell Transfer

The effect of CD226 (i.e. DNAM-1) expression on T cells used in adoptive cell transfer (ACT) immunotherapy was assessed in mice harboring 616F10 melanomas. ACT immunotherapy was performed as described in Example 1. Briefly, CD226⁺ (DNAM-1⁺) and DNAM-1⁻ (CD226⁻) melanoma-specific CD8+ T cells were sorted from the spleens of pmel1-TCRtg mice. These T cells recognize the melanocytic lineage antigen gp100 and are able to recognize and destroy melanoma cells. After a single dose of cyclophosphamide, DNAM-1⁺ or DNAM-1⁻pmel1 T cells, along with an adenoviral vaccine for gp100 (V), followed by three intratumoral injections of immune stimulatory nucleic acids (I, CpG+polyI:C). As shown in FIG. 5A, DNAM-1⁻ T cells were significantly less effective in controlling B16F10 melanoma compared to DNAM-1⁺ T cells, indicating that the efficacy of ACT immunotherapy largely depends on DNAM-1+ T cells.

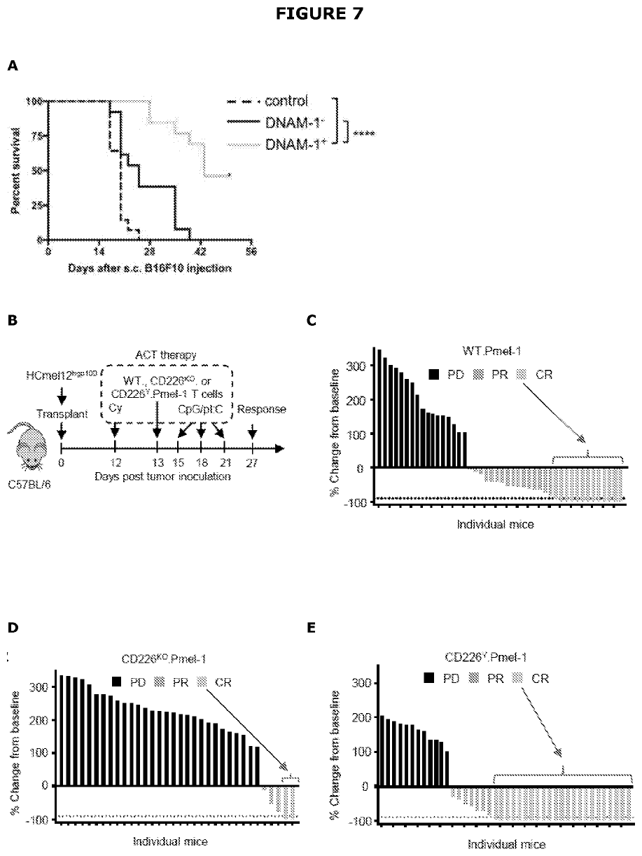

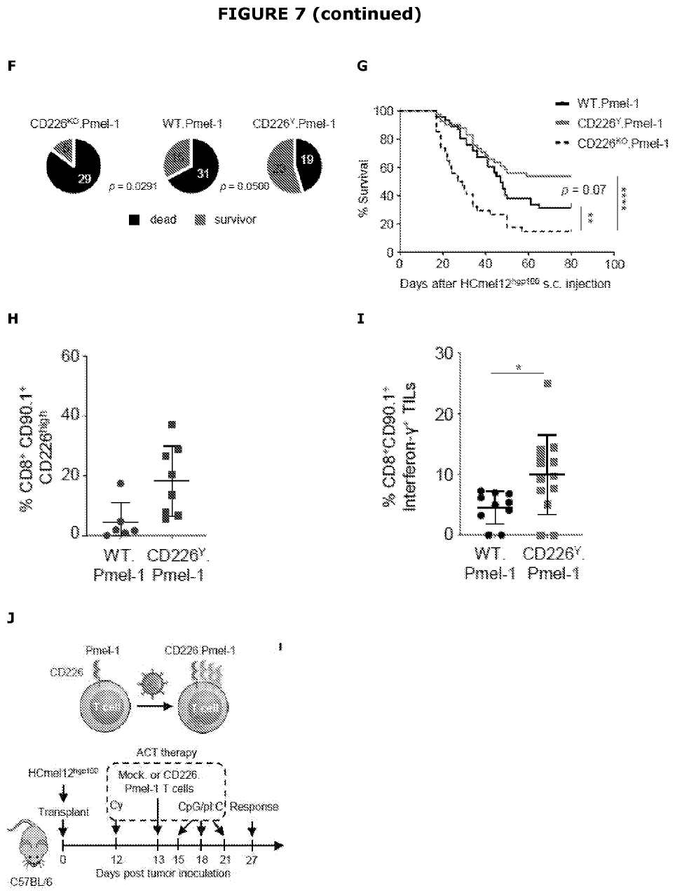

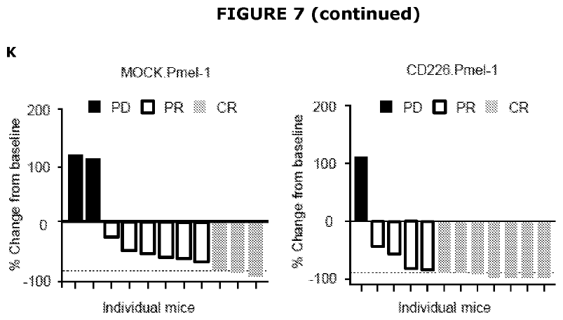

In a subsequent study, melanoma-bearing WT mice were treated with ACT therapy using WT.Pmel-1, CD226^{KO}.Pmel-1 or CD226^{Y}.Pmel-1 T cells. For this, WT mice were injected s.c. with HCmel12^{hgp100} melanomas, a mouse melanoma cell line derived from a primary Hgf-Cdk4 melanoma engineered to express the high affinity antigen hgp100 recognised by Pmel-1 T cells. Once tumors reached ˜5 mm in diameter mice were treated with a single dose cyclophosphamide for chemotherapeutic preconditioning. The next day cohorts of mice received either WT.Pmel-1, CD226^{KO}.Pmel-1 or CD226^{Y}.Pmel-1 T cells followed by innate immune stimulation (FIG. 76). Adoptive transfer of WT.Pmel-1 T cells induced robust anti-tumor immunity with 13 of 44 complete responders (CR, >90% of tumor shrinkage on day 14 after therapy compared to baseline) (FIG. 7C). In contrast, CD226^{KO}.Pmel-1 T cells largely failed to induce CRs (2 of 34) (FIG. 7D). Corroborating the previous findings, ACT immunotherapy using CD226^{Y}.Pmel-1 T cells was superior than transfer of WT.Pmel-1 T cells (23 of 42 CRs), resulting in significantly increased numbers of long-term surviving mice and improved survival (FIG. 7E-G). Flow cytometric analyses of tumor infiltrating Pmel-1 T cells revealed that CD226^{Y}.Pmel-1 T cells showed increased CD226 expression associated with increased IFN-γ and TNF-α production compared to WT.Pmel-1 T cells (FIG. 7H and I). Similar to the B16F10 melanoma model, no stringent correlation between the expression of inhibitory immune receptors and CD226 in adoptively transferred WT.Pmel-1 T cells was observed (data not shown).

As CD226 surface expression in CD8+ T cells correlated with superior tumor control, it was hypothesized that overexpression of CD226 might be a rational strategy to improve adoptive cell transfer therapies. To therapeutically increase CD226 expression, WT.Pmel-1 T cells were transduced with control (MOCK.Pmel-1) or CD226-encoding retroviral vectors (CD226.Pmel-1) prior to adoptive transfer into HCmel12hgp100 bearing WT mice using the ACT protocol (FIG. 7J). Indeed, overexpression of CD226 in Pmel-1 T cells improved therapeutic efficacy and increased the number of CR compared to MOCK.Pmel-1 T cells (FIG. 7K).

### Example 7

### Importance of DNAM-1 in Immune Checkpoint Inhibitor Therapy

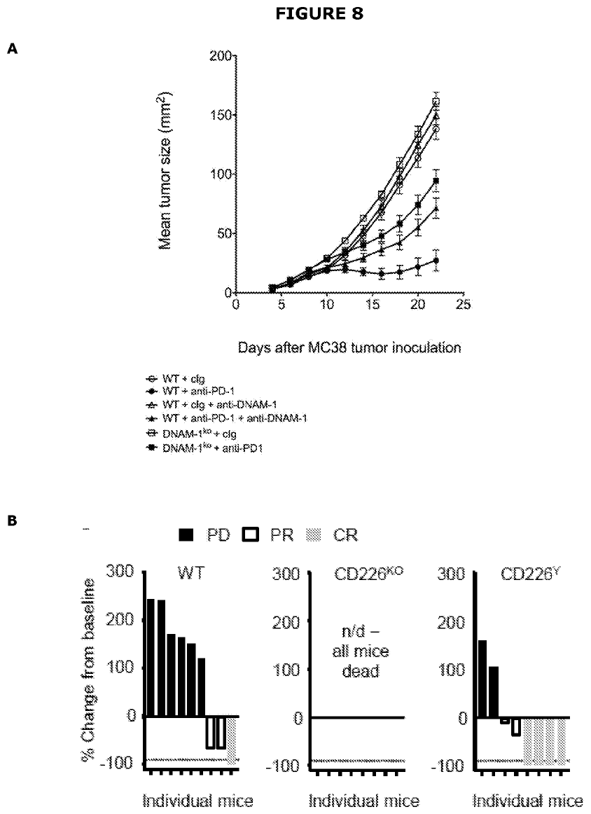

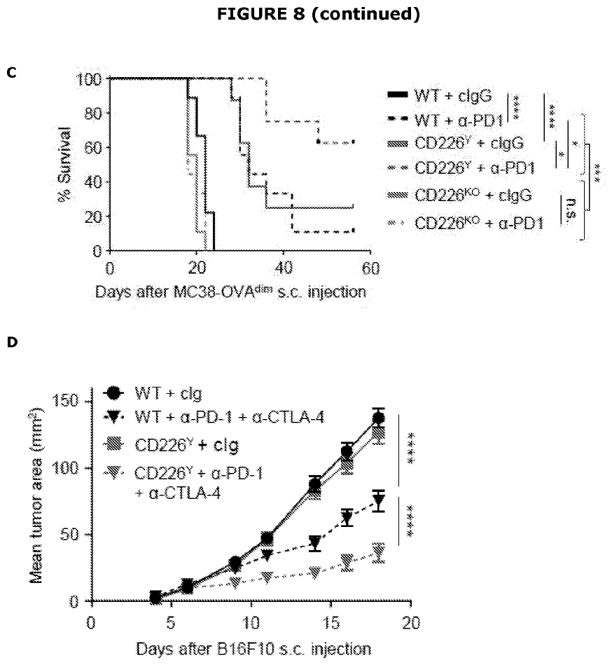

The importance of DNAM-1 expression in immune checkpoint inhibitor therapy was assessed in WT and CD226^{KO} (DNAM-1^{KO}) mice harbouring MC38 colon adenocarcinomas. Briefly, and as described in Example 1, the mice were injected s.c. with MC38 colon adenocarcinoma cells before being administered control Ig (cIg) or anti-PD1 (RMP1-14) mAb on days 10, 12, 14, and 16 after tumor inoculation. Groups of WT mice received either cIg or anti-CD226 mAb on days 9, 10, 14, 17, 20, and 24. As shown in FIG. 8A, the efficacy of anti-PD1 antibodies was significantly reduced in CD226^{KO} mice and WT mice that received anti-DNAM-1 antibodies compared to WT mice that received anti-PD1 antibodies alone.

In a subsequent study, WT, CD226^{KO} or CD226^{Y} mice were injected with MC38-OVA^{dim} cells and treated with anti-PD1 immunotherapy. While CD226^{KO} mice completely failed to mount an anti-tumor response, CD226^{Y} mice treated with control IgG (cIgG) showed a similar response as WT mice treated with anti-PD1. Importantly, CD226^{Y} mice treated with anti-PD-1 had the best survival (FIGS. 8B and C). The importance of CD226 for ICB was also highlighted by improved efficacy of anti-PD1+anti-CTLA4 combination immunotherapy in the poorly immunogenic B16F10 model (FIG. 8D). Taken together our data demonstrated that CD226 surface expression in CD8⁺ T cells correlates with the efficacy of cancer immunotherapies. Thus, effective immune checkpoint inhibitor therapy requires DNAM-1.

### Example 8

### Correlation of DNAM-1 Expression with Effector Function in TILs

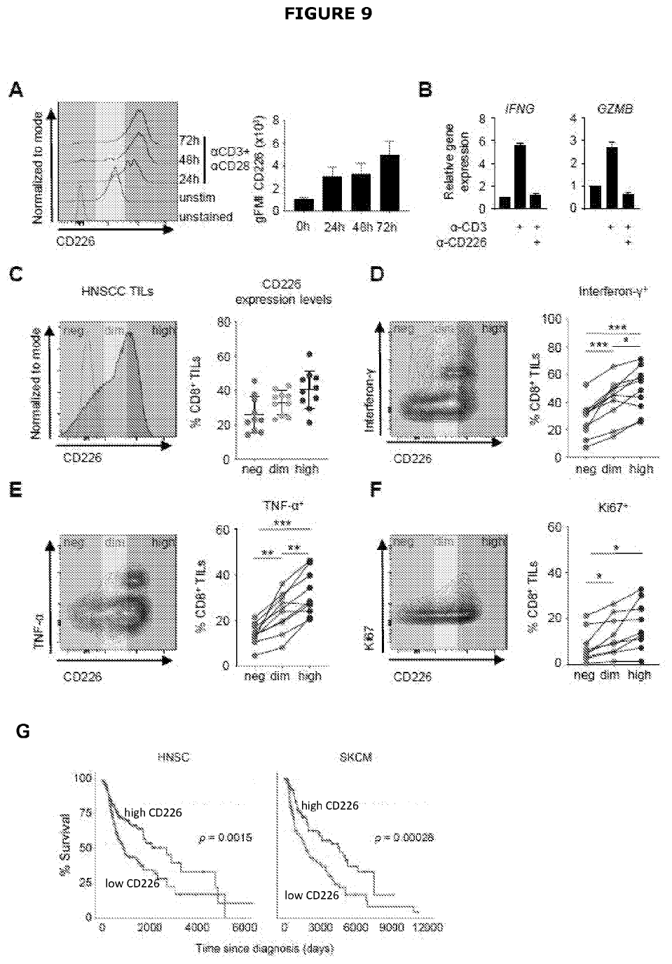

Using preclinical mouse models, DNAM-1 (i.e. CD226) was shown to be (a) downregulated in tumor infiltrating CD8⁺ T cells, (b) important for CD8⁺ T cell effector function, and (c) required for anti-tumor immunity and immunotherapy (see above). Studies were then performed to confirm that human CD8⁺ T cells isolated from PBMCs of healthy donors displayed upregulation of CD226 upon activation, in line with the results obtained from mice (FIG. 9A). In contrast to mice, a variable, but significant proportion of CD226 negative T cells (˜20%) was observed in the blood of healthy volunteers. To assess the importance of CD226 for T cell function, RNA expression analyses of PBMCs activated in the presence or absence of CD226 blocking antibodies (clone DX11) was performed. In this assay IFNG and GZMB expression upon TCR-stimulation was largely dependent on CD226 (FIG. 9B). Next, CD226 expression in CD8⁺ T cells isolated from tumor tissue samples from Head and Neck Squamous Cell Carcinoma (HNSCC) patients was assessed. Similar to mice, human tumor infiltrating CD8⁺ T cells showed variable surface expression of CD226 (FIG. 9C). Flow cytometric analysis of ex vivo stimulated CD8⁺ TILs showed a significant correlation between CD226 surface expression and effector function as evidenced by increased IFN-γ, TNF-α, Ki67, and CD107a staining (FIG. 9D-F). Importantly, CD226 gene expression was significantly associated with improved survival in HNSCC (HNSC) and cutaneous melanoma (SKCM) cohorts from the TCGA database (FIG. 9G). In these data sets, a significant correlation between CD226 gene expression and CD8B, IFNG, and GZMB, but not with NCAM1 (CD56) a classical NK cell marker, was observed (data not shown). Interestingly, a slight but significant inverse correlation between CD226 and PVR (CD155), but not NECTIN2 (CD112) gene expression, was also observed (data not shown).

### Example 9

### CD155 Binding Mediates DNAM-1 Downregulation in Human CD8+ T Cells

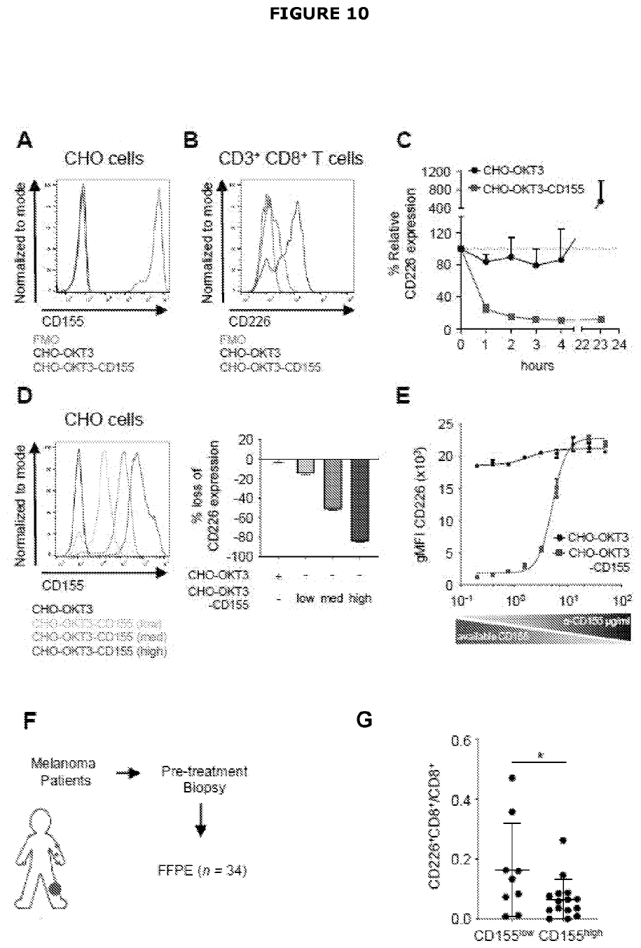

In mice, CD155 was identified as a major driver of DNAM-1 (CD226) downregulation in CD8⁺ T cells. To assess the impact of CD155 for CD226 surface expression in human CD8⁺ T cells, CHO cells stably expressing OKT3 and high levels of hCD155 were generated (FIG. 10A). When pre-activated human CD8⁺ T cells were incubated with CHO-OKT3 cells, increased CD226 surface expression was observed, while CD226 surface expression was substantially reduced in co-cultures with CHO-OKT3-CD155 cells (FIG. 10B). In fact, time-course analyses revealed that within 1 hour after the start of the co-culture the majority of CD8⁺ T cells had lost CD226 surface expression (FIG. 10C). To corroborate these findings and assess whether the amount of CD155 affects CD226 downregulation, CHO-OKT3 cells expressing various levels of CD155 were generated. Indeed, a CD155 dose-dependent CD226 downregulation was observed in the co-culture assays (FIG. 10D). In another set of experiments, increasing amounts of CD155 blocking antibodies were added to the co-cultures, validating the specific role for CD155 in CD226 downregulation in human CD8⁺ T cells in vitro (FIG. 10E). Thus, it was hypothesised that CD8⁺ TILs in cancer patients should express lower levels of CD226 in a CD155 high TME. To test this, 34 FFPE-samples from a well-annotated cohort of melanoma patients were stained for CD155 (FIG. 10F). Immunohistochemistry for CD155 revealed a subgroup of patients showing absent/low (n=9) or high (n=15) expression levels of CD155 (data not shown). Subsequent multiplex immunohistofluorescence (IHF) analyses confirmed that the ratio of CD226⁺CD8⁺ of total CD8⁺ T cells in the TME was negatively correlated with CD155 expression levels (FIG. 10H). In summary, this data supported the idea that tumor cell CD155 mediates downregulation of CD226 surface expression in tumor infiltrating CD8⁺ T cells.

### Example 10

### CD226 Surface Expression in CD8+ TILs Correlates with Response to ICB in Melanoma Patients

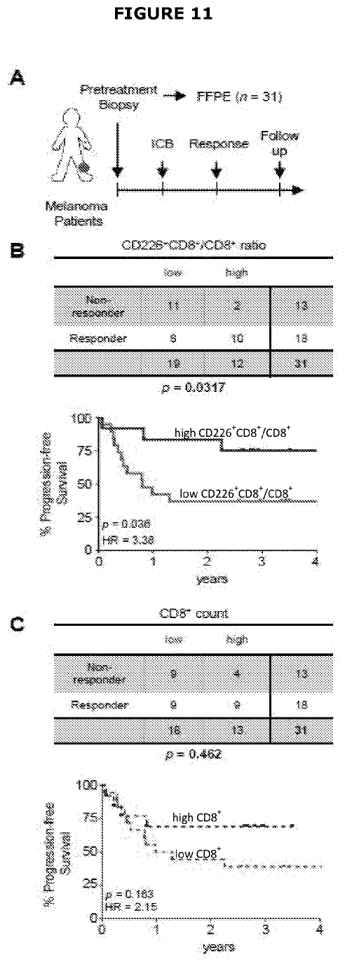

Since increased CD226 surface expression improves T cell effector function and the efficacy of cancer immunotherapies in pre-clinical mouse models, it was next asked if the response to ICB in human melanoma patients is dependent on the presence of CD226⁺CD8⁺ TILs. For this, the number of tumor infiltrating CD226⁺CD8⁺ T cells per total CD8⁺ T cells were detected using multiplex IHF in 31 pre-ICB treatment FFPE samples from a well-annotated cohort of melanoma patients (FIG. 11A). Indeed, the ratio of CD226⁺CD8⁺ in CD8⁺ T cells was significantly associated with response to ICB (FIG. 11B) and improved progression-free (PFS, HR 3.38; p=0.036) (FIG. 116). A similar trend, although statistically not significant was observed for overall survival (data not shown). Notably, this stratification of patients was not explained by total CD8⁺ T cell counts as neither response to ICB, PFS nor OS was significantly correlated with high CD8⁺ T cell counts (FIG. 11C). Thus, the data suggest that staining for CD226 could identify highly functional CD8⁺ T cells within the TME and improve the prediction of response to ICB. Overall, it was demonstrated that CD226 surface expression in CD8⁺ T cells is associated with effective anti-tumor immunity and cancer immunotherapy in cancer patients. Thus, CD155-induced downregulation of CD226 represents a novel and underrated resistance mechanism used by tumors to escape the immune system.

### Example 11

### Involvement of CBL-B in DNAM-1 Surface Expression in T Cells

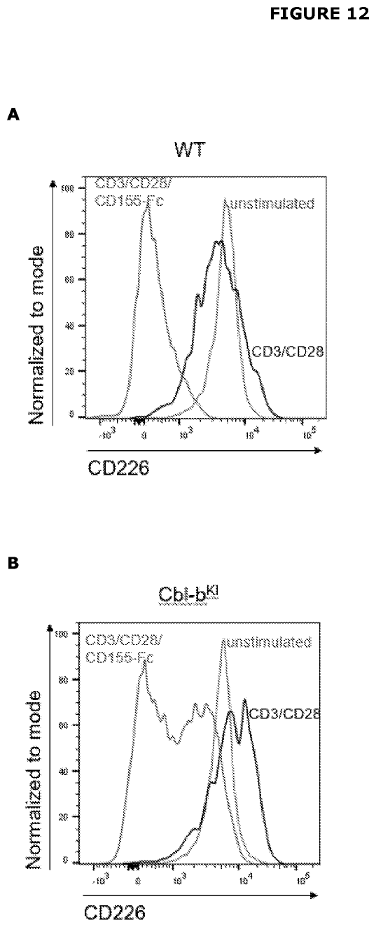

To assess whether the Ubiquitin ligase E3 Cbl-2 is involved in DNAM-1 ubiquitination and internalization, CD8⁺ T cells from the spleens of wild-type mice or mice harbouring a point mutation in the CBL-B gene resulting in abrogation of the ubiquitin ligase function (Cbl-b^{KI} mice) were assessed for DNAM-1 (CD226) surface expression following stimulation with CD3/CD28 beads or CD3/CD28/CD155-Fc beads for 16 h in IL-2 (50 IU/ml hIL-2) containing cRPMI media. As shown in FIG. 12, Cbl-b^{KI} mice are partially resistant to CD155-mediated CD226 downregulation.

### Example 12

### Overexpression of Modified DNAM-1 in T Cells

Several nucleic acid constructs encoding wild-type and modified DNAM-1 were synthesised and subcloned into the retroviral expression vector pMSCV-IRES-GFP II (Hoist et al. 2006, Nat Protoc. 1(1):406-17). Following production of retroviral vectors containing the DNAM-1 constructs, the vectors will be transduced into Pmel-1 T cells in vitro and cells will be purified based on GFP-expression. The impact of each DNAM-1 construct will be assessed in vitro by measuring T cell proliferation and cytokine production. Retrovirally-transduced pmel-1 T cells will also be used in the ACT immunotherapy model to assess the impact of overexpression of individual DNAM-1 constructs.

The constructs included polynucleotides encoding wild-type mouse DNAM-1; DNAM-1 No IgG1, which encodes a polypeptide lacking the IgG1 domain, i.e. lacking aa 30-127 of the wild-type DNAM-1 set forth in SEQ ID NO:3, thereby comprising aa 1-29 and 128-333 of wild-type DNAM-1; DNAM-1 No IgG1+IgG2, which encodes a polypeptide lacking the IgG1 and IgG2 domain, i.e. lacking aa 30-127 and 138-237 of the wild-type DNAM-1 set forth in SEQ ID NO:3, thereby comprising aa 1-29 and 128-137 and 128-333 of wild-type DNAM-1; DNAM-1 No intracellular, which encodes a polypeptide lacking the intracellular (or cytoplasmic) domain, i.e. lacking aa 275-333 of the wild-type DNAM-1 set forth in SEQ ID NO:3, thereby comprising aa 1-274 of wild-type DNAM-1; DNAM-1 S326A, which encodes a polypeptide comprising a S326A mutation relative to the wild-type DNAM-1 set forth in SEQ ID NO:3; and DNAM-1 Y319A/S326A, which encodes a polypeptide comprising a Y319A mutation and a S326A mutation relative to the wild-type DNAM-1 set forth in SEQ ID NO:3.

Wild-type mouse DNAM-1 (polynucleotide):
(SEQ ID NO: 11)
atggcttatgttacttggcttttggctattcttcatgtgcacaaagcactgtgtgaagagacattgtgggacacaacagttcggctttctgagact
atgactctggaatgtgtatatccattgacgcataacttaacccaggtggagtggaccaagaacactggcacaaagacagtgagcatagcagt
ttacaaccctaaccataatatgcatatagaatctaactacctccatagagtacacttcctaaactcaacagtggggttccgcaacatgagccttt
ccttttacaatgcctcagaagcagacattggcatctactcctgcttgtttcatgctttcccaaatggaccttgggaaaagaagataaaagtagtc
tggtcagatagttttgagatagcagcaccctcggatagctacctgtctgcagaacctggacaagatgtcacactcacttgccagcttccaagg
acttggccagtgcaacaagtcatatgggaaaaagtccagccccatcaggtagacatcttagcttcctgtaacctatctcaagagacaagatac
acttcaaagtacctaagacaaacaaggagcaactgtagccaggggagcatgaagagcatcctcatcattccaaatgccatggccgctgact
caggactttacagatgtcgctcagaggccattacaggaaaaaacaagtcctttgtcataaggctgatcataactgatggtggaaccaataaa
cattttatccttcccatcgttggagggttagtttcactgttacttgtcatcctaattatcatcattttcattttatataacaggaagagacggagaca
ggtgagaattccacttaaagagcccagggataaacagagtaaggtagccaccaactgcagaagtcctacttctcccatccagtctacagatg
atgaaaaagaggacatttatgtaaactatccaactttctctcgaagaccaaaaccaagactctaa
Wildtype mouse DNAM-1 (polypeptide):
(SEQ ID NO: 3)
MAYVTWLLAILHVHKALCEETLWDTTVRLSETMTLECVYPLTHNLTQVEWTKNTGTKTVSIAVYNPNHNMHIESN
YLHRVHFLNSTVGFRNMSLSFYNASEADIGIYSCLFHAFPNGPWEKKIKVVWSDSFEIAAPSDSYLSAEPGQDVT
LTCQLPRTWPVQQVIWEKVQPHQVDILASCNLSQETRYTSKYLRQTRSNCSQGSMKSILIIPNAMAADSGLYRC
RSEAITGKNKSFVIRLIITDGGTNKHFILPIVGGLVSLLLVILIIIIFILYNRKRRRQVRIPLKEPRDKQSKVATNCRSP
TSPIQSTDDEKEDIYVNYPTFSRRPKPRL
DNAM-1 No IgG1 (polynucleotide):
(SEQ ID NO: 12)
atggcttatgttacttggcttttggctattcttcatgtgcacaaagcactgtgtgaagagacattgtgggacacaacagttcggctttctgatagt
tttgagatagcagcaccctcggatagctacctgtctgcagaacctggacaagatgtcacactcacttgccagcttccaaggacttggccagtgc
aacaagtcatatgggaaaaagtccagccccatcaggtagacatcttagcttcctgtaacctatctcaagagacaagatacacttcaaagtacc
taagacaaacaaggagcaactgtagccaggggagcatgaagagcatcctcatcattccaaatgccatggccgctgactcaggactttacag
atgtcgctcagaggccattacaggaaaaaacaagtcctttgtcataaggctgatcataactgatggtggaaccaataaacattttatccttccc
atcgttggagggttagtttcactgttacttgtcatcctaattatcatcattttcattttatataacaggaagagacggagacaggtgagaattcca
cttaaagagcccagggataaacagagtaaggtagccaccaactgcagaagtcctacttctcccatccagtctacagatgatgaaaaagagg
acatttatgtaaactatccaactttctctcgaagaccaaaaccaagactctaa
DNAM-1 No IgG1 (polypeptide):
(SEQ ID NO: 13)
MAYVTWLLAILHVHKALCEETLWDTTVRLSDSFEIAAPSDSYLSAEPGQDVTLTCQLPRTWPVQQVIWEKVQPH
QVDILASCNLSQETRYTSKYLRQTRSNCSQGSMKSILIIPNAMAADSGLYRCRSEAITGKNKSFVIRLIITDGGTN
KHFILPIVGGLVSLLLVILIIIIFILYNRKRRRQVRIPLKEPRDKQSKVATNCRSPTSPIQSTDDEKEDIYVNYPTFSR
RPKPRL
DNAM-1 No IgG1 + IgG2 (polynucleotide):
(SEQ ID NO: 14)
atggcttatgttacttggcttttggctattcttcatgtgcacaaagcactgtgtgaagagacattgtgggacacaacagttcggctttctgatagt
tttgagatagcagcaccctcgataaggctgatcataactgatggtggaaccaataaacattttatccttcccatcgttggagggttagtttcact
gttacttgtcatcctaattatcatcattttcattttatataacaggaagagacggagacaggtgagaattccacttaaagagcccagggataaa
cagagtaaggtagccaccaactgcagaagtcctacttctcccatccagtctacagatgatgaaaaagaggacatttatgtaaactatccaact
ttctctcgaagaccaaaaccaagactctaa
DNAM-1 No IgG1 + IgG2 (polypeptide):
(SEQ ID NO: 15)
MAYVTWLLAILHVHKALCEETLWDTTVRLSDSFEIAAPSIRLIITDGGTNKHFILPIVGGLVSLLLVILIIIIFILYNRK
RRRQVRIPLKEPRDKQSKVATNCRSPTSPIQSTDDEKEDIYVNYPTFSRRPKPRL
DNAM-1 No intracellular (polynucleotide):
(SEQ ID NO: 16)
atggcttatgttacttggcttttggctattcttcatgtgcacaaagcactgtgtgaagagacattgtgggacacaacagttcggctttctgagact
atgactctggaatgtgtatatccattgacgcataacttaacccaggtggagtggaccaagaacactggcacaaagacagtgagcatagcagt
ttacaaccctaaccataatatgcatatagaatctaactacctccatagagtacacttcctaaactcaacagtggggttccgcaacatgagccttt
ccttttacaatgcctcagaagcagacattggcatctactcctgcttgtttcatgctttcccaaatggaccttgggaaaagaagataaaagtagtc
tggtcagatagttttgagatagcagcaccctcggatagctacctgtctgcagaacctggacaagatgtcacactcacttgccagcttccaagg
acttggccagtgcaacaagtcatatgggaaaaagtccagccccatcaggtagacatcttagcttcctgtaacctatctcaagagacaagatac
acttcaaagtacctaagacaaacaaggagcaactgtagccaggggagcatgaagagcatcctcatcattccaaatgccatggccgctgact
caggactttacagatgtcgctcagaggccattacaggaaaaaacaagtcctttgtcataaggctgatcataactgatggtggaaccaataaa
cattttatccttcccatcgttggagggttagtttcactgttacttgtcatcctaattatcatcattttcattttaa
DNAM-1 No intracellular (polypeptide):
(SEQ ID NO:16)
MAYVTWLLAILHVHKALCEETLWDTTVRLSETMTLECVYPLTHNLTQVEWTKNTGTKTVSIAVYNPNHNMHIESN
YLHRVHFLNSTVGFRNMSLSFYNASEADIGIYSCLFHAFPNGPWEKKIKVVWSDSFEIAAPSDSYLSAEPGQDVT
LTCQLPRTWPVQQVIWEKVQPHQVDILASCNLSQETRYTSKYLRQTRSNCSQGSMKSILIIPNAMAADSGLYRC
RSEAITGKNKSFVIRLIITDGGTNKHFILPIVGGLVSLLLVILIIIIFIL
DNAM-1 S326A (polynucleotide):
(SEQ ID NO: 17)
atggcttatgttacttggcttttggctattcttcatgtgcacaaagcactgtgtgaagagacattgtgggacacaacagttcggctttctgagact
atgactctggaatgtgtatatccattgacgcataacttaacccaggtggagtggaccaagaacactggcacaaagacagtgagcatagcagt
ttacaaccctaaccataatatgcatatagaatctaactacctccatagagtacacttcctaaactcaacagtggggttccgcaacatgagccttt
ccttttacaatgcctcagaagcagacattggcatctactcctgcttgtttcatgctttcccaaatggaccttgggaaaagaagataaaagtagtc
tggtcagatagttttgagatagcagcaccctcggatagctacctgtctgcagaacctggacaagatgtcacactcacttgccagcttccaagg
acttggccagtgcaacaagtcatatgggaaaaagtccagccccatcaggtagacatcttagcttcctgtaacctatctcaagagacaagatac
acttcaaagtacctaagacaaacaaggagcaactgtagccaggggagcatgaagagcatcctcatcattccaaatgccatggccgctgact
caggactttacagatgtcgctcagaggccattacaggaaaaaacaagtcctttgtcataaggctgatcataactgatggtggaaccaataaa
cattttatccttcccatcgttggagggttagtttcactgttacttgtcatcctaattatcatcattttcattttatataacaggaagagacggagaca
ggtgagaattccacttaaagagcccagggataaacagagtaaggtagccaccaactgcagaagtcctacttctcccatccagtctacagatg
atgaaaaagaggacatttatgtaaactatccaactttcgctcgaagaccaaaaccaagactctaa
DNAM-1 S326A (polypeptide):
(SEQ ID NO: 18)
MAYVTWLLAILHVHKALCEETLWDTTVRLSETMTLECVYPLTHNLTQVEWTKNTGTKTVSIAVYNPNHNMHIESN
YLHRVHFLNSTVGFRNMSLSFYNASEADIGIYSCLFHAFPNGPWEKKIKVVWSDSFEIAAPSDSYLSAEPGQDVT
LTCQLPRTWPVQQVIWEKVQPHQVDILASCNLSQETRYTSKYLRQTRSNCSQGSMKSILIIPNAMAADSGLYRC
RSEAITGKNKSFVIRLIITDGGTNKHFILPIVGGLVSLLLVILIIIIFILYNRKRRRQVRIPLKEPRDKQSKVATNCRSP
TSPIQSTDDEKEDIYVNYPTFARRPKPRL
DNAM-1 Y319A/S326A (polynucleotide):
(SEQ ID NO: 19)
atggcttatgttacttggcttttggctattcttcatgtgcacaaagcactgtgtgaagagacattgtgggacacaacagttcggctttctgagact
atgactctggaatgtgtatatccattgacgcataacttaacccaggtggagtggaccaagaacactggcacaaagacagtgagcatagcagt
ttacaaccctaaccataatatgcatatagaatctaactacctccatagagtacacttcctaaactcaacagtggggttccgcaacatgagccttt
ccttttacaatgcctcagaagcagacattggcatctactcctgcttgtttcatgctttcccaaatggaccttgggaaaagaagataaaagtagtc
tggtcagatagttttgagatagcagcaccctcggatagctacctgtctgcagaacctggacaagatgtcacactcacttgccagcttccaagg
acttggccagtgcaacaagtcatatgggaaaaagtccagccccatcaggtagacatcttagcttcctgtaacctatctcaagagacaagatac
acttcaaagtacctaagacaaacaaggagcaactgtagccaggggagcatgaagagcatcctcatcattccaaatgccatggccgctgact
caggactttacagatgtcgctcagaggccattacaggaaaaaacaagtcctttgtcataaggctgatcataactgatggtggaaccaataaa
cattttatccttcccatcgttggagggttagtttcactgttacttgtcatcctaattatcatcattttcattttatataacaggaagagacggagaca
ggtgagaattccacttaaagagcccagggataaacagagtaaggtagccaccaactgcagaagtcctacttctcccatccagtctacagatg
atgaaaaagaggacattgctgtaaactatccaactttcgctcgaagaccaaaaccaagactctaa
DNAM-1 Y319A/S326A (polypeptide):
(SEQ ID NO: 20)
MAYVTWLLAILHVHKALCEETLWDTTVRLSETMTLECVYPLTHNLTQVEWTKNTGTKTVSIAVYNPNHNMHIESN
YLHRVHFLNSTVGFRNMSLSFYNASEADIGIYSCLFHAFPNGPWEKKIKVVWSDSFEIAAPSDSYLSAEPGQDVT
LTCQLPRTWPVQQVIWEKVQPHQVDILASCNLSQETRYTSKYLRQTRSNCSQGSMKSILIIPNAMAADSGLYRC
RSEAITGKNKSFVIRLIITDGGTNKHFILPIVGGLVSLLLVILIIIIFILYNRKRRRQVRIPLKEPRDKQSKVATNCRSP
TSPIQSTDDEKEDIAVNYPTFARRPKPRL

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A T cell, comprising a modified DNAM-1 polypeptide, wherein:
the modified DNAM-1 polypeptide exhibits increased retention on the surface of the cell compared to a wild-type DNAM-1 polypeptide; and
the T cell is a human T cell.

2. The T cell of claim 1, wherein the DNAM-1 polypeptide comprises a modification of a tyrosine at a position corresponding to position 322 of SEQ ID NO:1.

3. The T cell of claim 2, wherein the modification is an amino acid substitution or deletion.

4. The T cell of claim 2 or claim 3, wherein the modification is a substitution of the tyrosine with a phenylalanine.

5. The T cell of any one of claims 1-4, wherein the DNAM-1 polypeptide comprises a modification of the AP-2 binding motif YXXF at positions corresponding to positions 325-328 of SEQ ID NO:1, wherein the modification abolishes the AP-2 binding motif YXXF.

6. The T cell of any one of claims 1-5, wherein:
the DNAM-1 polypeptide comprises an amino acid substitution or deletion of the tyrosine at the position corresponding to position 325 of SEQ ID NO:1;
the DNAM-1 polypeptide comprises an amino acid substitution or deletion of the phenylalanine at the position corresponding to position 328 of SEQ ID NO:1;
the DNAM-1 polypeptide comprises an amino acid insertion after any one of the positions corresponding to position 325, 326 or 327 of SEQ ID NO:1; and/or
the DNAM-1 polypeptide comprises deletion of one or more of the residues at positions corresponding to positions 326 and 327 of SEQ ID NO:1.

7. The T cell of any one of claims 1-6, wherein the DNAM-1 polypeptide comprises a modification of the AP-2 binding motif EXXXLF at positions corresponding to positions 282-287 of SEQ ID NO:1, wherein the modification abolishes the AP-2 binding motif EXXXLF.

8. The T cell of any one of claims 1-7, wherein:
the DNAM-1 polypeptide comprises an amino acid substitution or deletion of the glutamic acid at the position corresponding to position 282 of SEQ ID NO:1;
the DNAM-1 polypeptide comprises an amino acid substitution or deletion of the leucine at the position corresponding to position 286 of SEQ ID NO;
the DNAM-1 polypeptide comprises an amino acid substitution or deletion of the phenylalanine at the position corresponding to position 287 of SEQ ID NO:1;
the DNAM-1 polypeptide comprises an amino acid insertion after any one or more of the residues at positions corresponding to 282-286 of SEQ ID NO:1; and/or
the DNAM-1 polypeptide comprises a deletion of one or more of the residues at positions corresponding to positions 283, 284 and 285 of SEQ ID NO:1.

9. The T cell of any one of claims 1-8, wherein the DNAM-1 polypeptide comprises a modification of the Cbl-B binding motif ((D/N)XpY) at positions corresponding to positions 320-322 of SEQ ID NO:1, wherein the modification abolishes the Cbl-B binding motif.

10. The T cell of any one of claims 1-9, wherein the DNAM-1 polypeptide comprises an amino acid deletion or substitution of the aspartic acid at the position corresponding to position 320 of SEQ ID NO:1.

11. The T cell of any one of claims 1-10, wherein the DNAM-1 polypeptide comprises an amino acid insertion after the position corresponding to position 320 and/or 321 of SEQ ID NO:1.

12. The T cell of any one of claims 1-11, wherein the DNAM-1 polypeptide comprises an amino acid substitution or deletion of the lysine at the position corresponding to position 295.

13. The T cell of any one of claims 1-12, wherein the DNAM-1 polypeptide comprises an amino acid substitution or deletion the lysine at the position corresponding to position 333 of SEQ ID NO:1.

14. The T cell of claim 1, wherein the DNAM-1 polypeptide lacks all or a portion of the cytoplasmic domain.

15. The T cell of any one of claims 1 to 14, wherein the DNAM-1 polypeptide comprises all or a portion of the extracellular domain.

16. The T cell of any one of claims 1 to 15, wherein the DNAM-1 polypeptide comprises the IgG1 domain.

17. The T cell of any one of claims 1 to 16, wherein the DNAM-1 polypeptide comprises the IgG2 domain.

18. The T cell of any one of claims 1 to 17, wherein the DNAM-1 polypeptide comprises a sequence of amino acids set forth in any one of SEQ ID NOs:5-9 or 21-30, or a sequence having at least or about 90%, 91%, 92%, 93%, 94%, 95%, 96%, 97%, 98% or 99% sequence identity thereto, wherein the DNAM-1 polypeptide does not comprise the same sequence as a wild-type DNAM-1 polypeptide.

19. The T cell of any one of claims 1 to 18, wherein the T cell is a CD8⁺ T cell.

20. The T cell of any one of claims 1 to 19, wherein the T cell is a CD4⁺ T cell.

21. The T cell of any one of claims 1 to 20, wherein the T cell is an αβ T cell or a yγδ T cell.

22. The T cell of any one of claims 1 to 21, wherein the T cell is derived from primary human PBMCs isolated from a human subject.

23. The T cell of any one of claims 1 to 22, comprising a recombinant TCR and/or a chimeric antigen receptor (CAR).

24. The T cell of claim 23, wherein the CAR binds to a tumor antigen selected from among TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-1Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-Ia, MAGE-A1, legumain, HPV E6, E7, MAGEA1, ETV6-AML, sperm protein 17, XAGE1, Tie 2, MAD-CT-1, MAD-CT-2, Fos-related antigen 1, p53, p53 mutant, prostein, survivin and telomerase, PCTA-I/Galectin 8, MelanA/MART-1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

25. A pharmaceutical composition, comprising the T cell of any one of claims 1-24 and a pharmaceutically acceptable carrier.

26. The pharmaceutical composition of claim 25, further comprising a chemotherapeutic agent or an anti-infective agent.

27. The pharmaceutical composition of claim 26, wherein the chemotherapeutic agent is an immune checkpoint inhibitor.

28. The pharmaceutical composition of claim 27, wherein the immune checkpoint inhibitor is selected from among a CTLA-4, PD-1 and PD-L1 inhibitor.

29. The pharmaceutical composition of claim 26, wherein the anti-infective agent is selected from among an antibiotic, amebicide, antifungal, antiprotozoal, antimalarial, antituberculotic and antiviral.

30. A method for preparing a T cell population for adoptive cell therapy, comprising:
obtaining a sample of T cells from a subject;
selecting DNAM+ T cells from the sample; and
expanding the DNAM+ T cells to produce a T cell population for adoptive T cell therapy.

31. The method of claim 36, wherein the method comprises selecting DNAM+ CD8+ T cells.

32. The method of claim 36, wherein the method comprises selecting DNAM+ CD4+ T cells.

33. The method of any one of claims 36 to 38, further comprising engineering the DNAM⁺ T cells to express a CAR or a transgenic TCR.

34. A T cell population produced by the method of any one of claims 30 to 33.

35. A method of increasing immune function in a subject, comprising administering to the subject the T cell of any one of claims 1 to 24, the pharmaceutical composition of any one of claims 25 to 29 or the T cell population of claim 34.

36. A method for treating cancer in a subject, comprising administering to the subject the T cell of any one of claims 1 to 24, the pharmaceutical composition of any one of claims 25 to 29 or the T cell population of claim 34.

37. The method of claim 36, further comprising administering a chemotherapeutic agent to the subject.

38. The method of claim 37, wherein the chemotherapeutic agent is an immune checkpoint inhibitor.

39. The method of claim 38, wherein the immune checkpoint inhibitor is selected from among a CTLA-4, PD-1 and PD-L1 inhibitor.

40. The method of any one of claims 35 to 39, wherein the cancer is skin cancer (e.g., melanoma), lung cancer, breast cancer, ovarian cancer, gastric cancer, bladder cancer, pancreatic cancer, endometrial cancer, colon cancer, kidney cancer, esophageal cancer, prostate cancer, colorectal cancer, glioblastoma, head and neck cancer, neuroblastoma, or hepatocellular carcinoma.

41. The method of any one of claims 35 to 40, wherein the cancer is resistant to one or more immune checkpoint inhibitors prior to administration of the T cell or pharmaceutical composition.

42. The method of any one of claims 35 to 41, wherein the T cell is autologous.

43. The method of any one of claims 35 to 41, wherein the T cell is allogeneic.

44. A method for treating an infection in a subject, comprising administering to the subject the T cell of any one of claims 1 to 24, the pharmaceutical composition of any one of claims 25 to 29, or the T cell population of claim 40.

45. The method of claim 44, wherein the infection is with virus and/or bacteria.

46. The method of claim 44 or claim 45, wherein the infection is an acute infection.

47. The method of claim 44 or claim 45, wherein the infection is a chronic infection.

48. The method of any one of claims 44 to 47, further comprising administering an anti-infective agent to the subject.

49. The method of claim 48, wherein the anti-infective agent is selected from among an antibiotic, amebicide, antifungal, antiprotozoal, antimalarial, antituberculotic and antiviral.

50. The method of any one of claims 44 to 49, wherein the T cell is autologous.

51. The method of any one of claims 44 to 49, wherein the T cell is allogeneic.

52. Use of the T cell of any one of claims 1 to 24, the pharmaceutical composition of any one of claims 25 to 29 or the T cell population of claim 34, for the preparation of a medicament for treating cancer.

53. Use of the T cell of any one of claims 1 to 24, the pharmaceutical composition of any one of claims 25 to 29 or the T cell population of claim 34, for the preparation of a medicament for treating an infection.

54. Use of the T cell of any one of claims 1 to 24, the pharmaceutical composition of any one of claims 25 to 29 or the T cell population of claim 34, for the preparation of a medicament for enhancing immune function in a subject.

55. A method for assessing the immune function of a T cell or a population of T cells in a subject, comprising assessing the amount or level of DNAM-1 on the surface of a T cell or T cells in a population of T cells in a sample from the subject and comparing the amount or level of DNAM-1 on the surface of the T cell or T cells in the population of T cells in the sample from the subject to the amount or level of DNAM-1 on the surface of a T cell or T cells in a population of T cells in a control sample, or to a reference level.

56. The method of claim 55, wherein assessing the amount or level of DNAM-1 on the surface of T cells in a population of T cells in a sample comprises detecting the number or percentage of DNAM-1+ T cells in the population of T cells.

57. The method of claim 55 or 56, wherein the control sample comprises T cells with normal or effective immune function, and a reduced amount or level of DNAM-1 on the surface of a T cell or T cells in a population of T cells in the sample from the subject compared to the amount or level of DNAM-1 on the surface of a T cell in the control sample indicates that the immune function of the T cell or a populations of T cells in the subject is impaired or ineffective.

58. The method of any one of claims 55 to 57, comprising:
obtaining a sample from the subject, wherein the sample comprises a T cell or population of T cells;
contacting the sample with a binding agent that binds to DNAM-1 on the surface of a T cell; and
detecting the binding agent when bound to the T cell or T cells in the population of T cells to thereby assess the amount or level of DNAM-1 on the surface of the T cells or the number or percentage of DNAM+T cells in the sample from the subject.

59. The method of claim 58, wherein the binding agent is an anti-DNAM-1 antibody.

60. The method of any one of claims 55 to 59, wherein the subject has cancer or has an infection.

61. A method for predicting the likelihood that a subject with cancer will respond to therapy with an immune checkpoint inhibitor, comprising detecting the number or percentage of DNAM-1+ CD8+ T cells in a sample from the subject, and comparing the number or percentage of DNAM-1+ CD8+ cells in the sample from the subject to a reference level or amount.

62. The method of claim 61, wherein the percentage of DNAM-1+ CD8+ T cells as a percentage of total CD8+ T cells in the sample is detected.

63. The method of claim 61 or 62, wherein the sample is a tumour sample and the T cells are tumour infiltrating T cells.

64. A modified DNAM-1 polypeptide comprising a modification of the AP-2 binding motif YXXF at positions corresponding to positions 325-328 of SEQ ID NO:1, wherein the modification abolishes the AP-2 binding motif YXXF.

65. The modified DNAM-1 polypeptide of claim 64, wherein:
the DNAM-1 polypeptide comprises an amino acid substitution or deletion of the tyrosine at the position corresponding to position 325 of SEQ ID NO:1;
the DNAM-1 polypeptide comprises an amino acid substitution or deletion of the phenylalanine at the position corresponding to position 328 of SEQ ID NO:1;
the DNAM-1 polypeptide comprises an amino acid insertion after any one of the positions corresponding to position 325, 326 or 327 of SEQ ID NO:1; and/or
the DNAM-1 polypeptide comprises a deletion of one or more of the residues at positions corresponding to positions 326 and 327 of SEQ ID NO:1.

66. A modified DNAM-1 polypeptide comprising a modification of the AP-2 binding motif EXXXLF at positions corresponding to positions 282-287 of SEQ ID NO:1, wherein the modification abolishes the AP-2 binding motif EXXXLF.

67. The modified DNAM-1 polypeptide of claim 66, wherein:
the DNAM-1 polypeptide comprises an amino acid substitution or deletion of the glutamic acid at the position corresponding to position 282 of SEQ ID NO:1;
the DNAM-1 polypeptide comprises an amino acid substitution or deletion of the leucine at the position corresponding to position 286 of SEQ ID NO;
the DNAM-1 polypeptide comprises an amino acid substitution or deletion of the phenylalanine at the position corresponding to position 287 of SEQ ID NO:1;
the DNAM-1 polypeptide comprises an amino acid insertion after any one or more of the residues at positions corresponding to 282-286 of SEQ ID NO:1; and/or
the DNAM-1 polypeptide comprises a deletion of one or more of the residues at positions corresponding to positions 283, 284 and 285 of SEQ ID NO:1.

68. A modified DNAM-1 polypeptide comprising a modification of the Cbl-b binding motif ((D/N)XpY) at positions corresponding to positions 320-322 of SEQ ID NO:1, wherein the modification abolishes the Cbl-6 binding motif.

69. The modified DNAM-1 polypeptide of claim 68, wherein the DNAM-1 polypeptide comprises an amino acid deletion or substitution of the aspartic acid at the position corresponding to position 320 of SEQ ID NO:1.

70. The modified DNAM-1 polypeptide of claim 68 or 69, wherein the DNAM-1 polypeptide comprises an amino acid insertion after the position corresponding to position 320 and/or 321 of SEQ ID NO:1.

71. A modified DNAM-1 polypeptide comprising a modification (e.g. an amino acid substitution or deletion) of the lysine at the position corresponding to position 295 and/or the lysine at the position corresponding to position 333 of SEQ ID NO:1.

72. The modified DNAM-1 polypeptide of any one of claims 64 to 71 having increased surface retention when expressed in T cell compared to a wild-type DNAM-1 polypeptide when expressed in a T cell.

73. The modified DNAM-1 polypeptide of any one of claims 64-72, comprising a sequence of amino acids set forth in any one of SEQ ID NOs:5-9 or 21-30, or a sequence having at least or about 90%, 91%, 92%, 93%, 94%, 95%, 96%, 97%, 98% or 99% sequence identity thereto, wherein the DNAM-1 polypeptide does not comprise the same sequence as a wild-type DNAM-1 polypeptide.
